(19) United States
(12) Patent Application Publication
(10) Pub. No.: US 2025/0280776 A1
(43) Pub. Date: Sep. 11, 2025

(54) CORN ELITE EVENT MZIR260

(71) Applicant: Syngenta Crop Protection AG, Basel (CH)

(72) Inventors: Sumita Chaudhuri, RTP, NC (US); Andrew Noe, Clinton, IL (US); Liesel Joanna Schneider, Union City, TN (US); Betiana Paula Parody, Sao Paulo (BR); Yuki Matsuba, RTP, NC (US); Sonia Herrero, Raleigh, NC (US); Weining Gu, RTP, NC (US); Kian Eghrari Moraes, Uberlândia, Minas Gerais (BR); Anderson Bolzan, Uberlândia, Minas Gerais (BR); Mark Scott Rose, RTP, NC (US); Radhika Bartaula, RTP, NC (US); Tae-Jin Lee, RTP, NC (US); Ailing Zhou, RTP, NC (US)

(73) Assignee: Syngenta Crop Protection AG, Basel (CH)

(21) Appl. No.: 19/186,912

(22) Filed: Apr. 23, 2025

Related U.S. Application Data
(63) Continuation-in-part of application No. 19/073,189, filed on Mar. 7, 2025.
(60) Provisional application No. 63/562,517, filed on Mar. 7, 2024.

(51) Int. Cl. A01H 1/04; C12Q 1/6806; C12Q 1/686; C12Q 1/6869; C12Q 1/6895

(52) U.S. Cl. CPC A01H 1/045; C12Q 1/6806; C12Q 1/686; C12Q 1/6869; C12Q 1/6895; C12Q 2600/13

(57) ABSTRACT

A novel transgenic corn elite event designated MZIR260 is disclosed. The invention relates to DNA sequences of the recombinant constructs inserted into the corn genome and of genomic sequences flanking the insertion site that resulted in elite event MZIR260. The invention further relates to assays for detecting the presence of the DNA sequences of corn elite event MZIR260, to corn plants and corn seeds comprising the genotype thereof, and to methods for producing a corn plant by crossing a corn plant comprising the elite event MZIR260 genotype with itself or another corn variety.

### RELATED APPLICATION INFORMATION

This application is a Continuation-in-Part of U.S. patent application Ser. No. 19/073,189, filed Mar. 7, 2025, which claims priority to U.S. Provisional Application Ser. No. 63/562,517 filed Mar. 7, 2024, the contents of each of which are incorporated herein by reference in its entirety.

### STATEMENT REGARDING ELECTRONIC SUBMISSION OF A SEQUENCE LISTING

A sequence listing in XML format, submitted under 37 C.F.R. § 1.821, entitled “PAT-109813-US-CIP-1_SEQLIST.xml”, 120 kilobytes in size, generated on Apr. 14, 2025, and filed via Patent Center is provided in lieu of a paper copy. This sequence listing is hereby incorporated by reference into the specification for its disclosures.

### FIELD OF THE INVENTION

The invention relates generally to the field of plant molecular biology, plant transformation, and plant breeding. More specifically, the invention relates to insect resistant transgenic corn plants and to methods of detecting the presence of the corn plant DNA in a sample and compositions thereof.

### BACKGROUND

Plant pests are a major factor in the loss of the world's important agricultural crops. Each year, invasive insects cost the global economy around $70 billion (L. Gula, 2023, National Institute of Food and Agriculture, United States Department of Agriculture). Plant pests are a major factor in the loss of the world's important agricultural crops, including maize. Plant pests are mainly controlled by intensive applications of chemical pesticides. Good pest control can thus be reached, but these chemicals can sometimes also affect beneficial organisms. Another problem resulting from the wide use of chemical pesticides is the appearance of resistant insect varieties. This has been partially alleviated by various resistance management practices, but there is an increasing need for alternative pest control strategies. One such alternative includes the expression of foreign genes encoding insecticidal proteins in transgenic plants. This approach has provided an efficient means of protection against selected insect pests, and transgenic plants expressing insecticidal toxins have been commercialized, allowing farmers to reduce applications of chemical insecticides.

Bacillus thuringiensis (Bt) Cry proteins (also called 5-endotoxins) are proteins that form a crystalline matrix in Bacillus that are known to possess insecticidal activity when ingested by certain insects. Genes coding for Cry proteins have been isolated and their expression in crop plants have been shown to provide another tool for the control of economically important insect pests. Such transgenic plants expressing the Cry proteins have been commercialized, allowing farmers to reduce or augment applications of chemical insect control agents. Lepidopteran-active Cry proteins useful in transgenic plants include, for example, Cry1, Cry2, Cry3, Cry9, and the like, as well as vegetative insecticidal proteins such as Vip1, Vip2, Vip3, and the like.

Although the usage of transgenic plants expressing Cry proteins is another tool in the insect control toolbox, it is still susceptible to resistance breakdown. Insect pests that now have resistance against the Cry proteins expressed in certain transgenic plants are known. For example, fall armyworm (Spodoptera frugiperda) has documented field-evolved resistance to Cry1F, Cry1A.105 and Cry2A b2 in certain countries. As a result, there is a need for additional insecticidal proteins, and transgenic events effectively expressing these proteins, to address the resistance issues.

The expression of foreign genes in plants can be influenced by their chromosomal position, for example due to chromatin structure and/or the proximity of transcriptional regulation elements close to the integration site (See for example, Weising et al., 1988, “Foreign Genes in Plants,” Ann. Rev. Genet. 22:421-477). A high-quality transgenic event is preferred to not be in a promoter or gene region of the genome. A high-quality transgenic event also should not have adverse effects on the agronomic performance of the transgenic plant. Additionally, a high-quality transgenic event is the result of a single copy of intact transgenic insertion, with little or no transgene rearrangement, and without extraneous heterologous DNA, such as DNA from the backbone of a vector used during the transformation process.

Therefore, it is common to produce hundreds of different events and screen those events for a single event that has desired molecular qualities and transgene expression levels and patterns for commercial purposes. The identified event which satisfies all criteria required for a high-quality event which may be used for commercial purposes is considered an elite event. The elite event is characterized by its exact genomic location, as it is that location which is responsible for the molecular qualities, transgene expression levels, and agronomic performance of the event. This elite event is useful for introgressing its transgene into other genetic backgrounds by sexual outcrossing using conventional breeding methods. Progeny of such crosses maintain the transgene expression characteristics of the original transformant. This strategy may be used to generate an infinite number of hybrids and varieties comprising the elite event and to ensure reliable transgene expression in each variety and hybrid.

Because a particular elite event is characterized by its genomic location, it would be advantageous to be able to detect its presence in order to determine whether progeny of a sexual cross contain the elite event. In addition, a method for detecting a particular elite event would be helpful for complying with regulations requiring the pre-market approval and labeling of foods derived from recombinant crop plants, for example. It is possible to detect the presence of a transgene by any nucleic acid detection method, including but not limited to thermal amplification (polymerase chain reaction (PCR)) using polynucleotide primers or DNA hybridization using nucleic acid probes. Typically, for the sake of simplicity and uniformity of reagents and methodologies for use in detecting a particular DNA construct that has been used for transforming various plant varieties, these detection methods generally focus on frequently used genetic elements, for example, promoters, terminators, and marker genes, because for many DNA constructs, the coding sequence region is interchangeable. As a result, these element-specific methods may not be useful for discriminating between transgenic constructs that differ only with reference to the coding sequence. In addition, such methods are not useful for discriminating between different events, particularly those produced using the same DNA construct. To solve this problem, at least some portion of the sequence of genomic DNA adjacent to the inserted heterologous DNA, the flanking sequence, needs to be known. In particular, the junction sequence between the flanking genomic sequence and the inserted transgene needs to be known, primarily to identify the presence of the intended event.

### SUMMARY

The present invention is directed to corn elite event MZIR260 and related compositions and methods. Elite event MZIR260 comprises an expression cassette for eCry1Gb.1Ig, which encodes the insecticidal trait protein eCry1Gb.1Ig, along with the selectable marker gene phosphomannose isomerase (PMI) (NCBI accession number M 15380.1). The invention further includes novel isolated nucleic acid sequences, namely the junction sequences, which are unique to elite event MZIR260 and are useful for identifying the transgenic corn comprising elite event MZIR260 and for detecting nucleic acids from elite event MZIR260 in a biological sample. The present invention also includes compositions and kits comprising the reagents for use in detecting these nucleic acids in a biological sample.

Corn elite event MZIR260 comprises an expression cassette for eCry1Gb.1Ig-03, which encodes the insecticidal trait protein eCry1Gb.1Ig. eCry1Gb.1Ig is useful in controlling insect pests, particularly Spodoptera spp. and Diatraea spp. insect pests, in corn plants comprising this event and progeny thereof. The invention also provides transgenic corn plants comprising elite event MZIR260 or nucleic acid molecules from elite event MZIR260, seed, cells, and tissues from transgenic corn plants comprising elite event MZIR260 or nucleic acid molecules from elite event MZIR260, and methods for producing a transgenic corn plant comprising elite event MZIR260 or nucleic acid molecules from elite event MZIR260, e.g., by crossing a corn plant (e.g., an inbred) comprising elite event MZIR260 with itself or another corn line of a different genotype. The transgenic MZIR260 corn plants of the invention may have all or essentially all of the morphological and physiological characteristics of the corresponding isogenic non-transgenic corn plant in addition to the insect resistance trait conferred upon the corn plant by elite event MZIR260. The invention also provides compositions and methods for detecting the presence of nucleic acids from elite event MZIR260 based on the DNA sequence of the recombinant expression cassettes inserted into the corn genome that resulted in the elite event MZIR260, and of genomic sequences flanking the insertion site or based on one of both of the junction sequences.

Compositions of the present invention comprise nucleic acid molecules, transgenic corn plants and seed of a transgenic corn plant comprising elite event MZIR260, an example of the transgenic seed deposited as NCMA Accession No. 202310010. In various embodiments, the transgenic seed or transgenic corn plant comprises SEQ ID NO: 1, SEQ ID NO: 2, SEQ ID NO: 3, SEQ ID NO: 4, SEQ ID NO: 5, or SEQ ID NO: 6.

Methods and compositions are further provided for detection of the corn elite event MZIR260. In various embodiments, the compositions comprise a pair of polynucleotide primers comprising a first polynucleotide primer and a second polynucleotide primer which function together in the presence of a corn elite event MZIR260 DNA template in a sample to produce an amplicon diagnostic for the corn elite event MZIR260. In certain embodiments, the corn elite event MZIR260 DNA template comprises SEQ ID NO:1, SEQ ID NO: 2, SEQ ID NO: 3, SEQ ID NO: 4, SEQ ID NO: 5 or SEQ ID NO: 6, or a complement thereof. In embodiments, the first polynucleotide primer comprises a nucleotide sequence selected from the group consisting of SEQ ID NO: 11, 17, 20, 28, and 32, or the complements thereof, and/or wherein the second polynucleotide primer comprises at least 10 contiguous nucleotides as set forth in SEQ ID NO: 9, or the complements thereof. In some embodiments, the second polynucleotide primer comprises a nucleotide sequence selected from the group consisting of SEQ ID NO: 12, 18, 21, 29, and 33, or the complements thereof, or comprises at least 10 contiguous nucleotides of SEQ ID NO: 7, or the complement thereof.

In some embodiments, the present invention comprises a method of detecting the presence of a nucleic acid molecule that is unique to elite event MZIR260 in a sample comprising corn nucleic acids, wherein the method comprises (a) providing a genomic DNA sample from a corn plant that comprises a nucleic acid molecule that is unique to elite event MZIR260; (b) combining the nucleic acid molecule with a pair of polynucleotide primers set forth herein, (c) performing a nucleic acid amplification reaction which results in an amplicon diagnostic for the corn elite event MZIR260; and (d) detecting the amplicon. In embodiments, the amplicon comprises a nucleic acid sequence comprising SEQ ID NO: 1, SEQ ID NO: 2, SEQ ID NO: 3, SEQ ID NO: 4, SEQ ID NO: 5 or SEQ ID NO: 6, or a complement thereof. In further embodiments, the method further comprises selecting a plant comprising the amplicon for the purposes of breeding corn comprising corn elite event MZIR260.

Also provided herein are methods of confirming the absence of a nucleic acid molecule that is unique to elite event MZIR260 in a sample comprising corn nucleic acids, wherein the method comprises (a) providing a genomic DNA sample from a corn plant, (b) combining the genomic DNA sample with a pair of polynucleotide primers set forth herein with a pair of polynucleotide primers that is complementary to a corn native genomic sequence as a positive control; (c) performing a nucleic acid amplification reaction which results in no amplicon specific to elite event MZIR260 and results in an amplicon specific to the corn native genome positive control; and (d) detecting the amplicon specific to the corn native genome positive control.

In various embodiments herein, the invention comprises a biological sample derived from elite event MZIR260, or a seed thereof, wherein the sample comprises a nucleotide sequence which comprises or is complementary to any one of SEQ ID NOs: 1-6. In certain embodiments, the biological sample is derived from a corn commodity product or is an extract of a biological sample, wherein the extract comprises a nucleotide sequence which comprises or is complementary to any one of SEQ ID NOs: 1-6. Further provided are methods of producing the corn commodity product encompassed herein. In certain embodiments, the biological sample or corn commodity product is a non-living (i.e., nonviable or devitalized, wherein the sample or commodity product is incapable of regenerating into a whole plant),

Also provided herein are methods of producing hybrid corn seed comprising corn elite event MZIR260, wherein the method comprises crossing the corn elite event MZIR260, or a seed thereof, with a second corn plant that is different from the corn elite event MZIR260 to produce hybrid corn seed and harvesting the hybrid corn seeds produced. The hybrid seed produced by the foregoing method, and plants grown therefrom, are also encompassed herein.

Various embodiments of the invention further comprise a kit for detecting the presence of corn elite event MZIR260 nucleic acids in a biological sample, the kit comprising at least one DNA molecule comprising a sufficient length of contiguous nucleotides that comprises or is complementary to any of SEQ ID NOs: 1-6 that functions as a DNA primer or probe specific for corn elite event MZIR260.

Further provided are methods for protecting a corn elite event MZIR260 plant against feeding damage by one or more pests (e.g., a Spodoptera spp. or a Diatraea spp.) by treating the seed of corn elite event MZIR260 or a transgenic corn plant grown therefrom with an insecticide.

In various embodiments herein, the invention comprises a process (and plants produced thereby) of introducing an additional trait into a corn elite event MZIR260 plant, wherein the process comprises (a) crossing a corn elite event MZIR260 with another maize plant that comprises an additional trait to produce hybrid progeny plants, (b) selecting hybrid progeny plants that have the additional trait to produce selected hybrid progeny plants, (c) crossing the selected progeny plants with the corn elite event MZIR260 parental plants to produce backcross progeny plants, (d) selecting for backcross progeny plants that have the additional trait to produce selected backcross progeny plants; and (e) repeating steps (c) and (d) at least two or more times to produce backcross progeny plants that comprise the additional trait and corn elite event MZIR260.

In various embodiments of the invention, corn elite event MZIR260 can be modified by introducing a modification into corn elite event MZIR260, wherein the modification may be a deletion, an insertion, a substitution, a duplication, or inversion or a combination thereof. The modification may comprise deletion of a portion or all of a selectable marker gene (e.g., a PMI coding sequence) present in the nucleic acid molecule. The modification may also comprise an insertion of a CRISPR-Cas nuclease PAM site within a region of corn elite event MZIR260 corresponding to any one or more of SEQ ID NOs: 1-6. In various embodiments, the modification is introduced using a nuclease or homologous recombination, or a combination thereof. In certain embodiments, the nuclease is a CRISPR-Cas nuclease. The methods herein further comprise producing a progeny plant by selfing or crossing the modified plant with another plant, thereby producing a modified transgenic progeny plant.

The invention comprises the following embodiments:

1. A seed of a transgenic corn plant comprising elite event MZIR260, an example of said seed deposited as NCMA Accession No. 202310010.

2. A transgenic corn plant comprising corn elite event MZIR260, an example of seed comprising said elite event MZIR260 deposited as NCMA Accession No. 202310010.

3. A transgenic corn plant comprising SEQ ID NO: 1, SEQ ID NO: 2, SEQ ID NO: 3, SEQ ID NO: 4, SEQ ID NO: 5, or SEQ ID NO: 6.

4. The transgenic corn plant or seed of embodiment 3, which comprises SEQ ID NO: 3, SEQ ID NO: 4, SEQ ID NO: 5, or SEQ ID NO: 6.

5. A seed of a transgenic corn plant comprising SEQ ID NO: 1, SEQ ID NO: 2, SEQ ID NO: 3, SEQ ID NO: 4, SEQ ID NO: 5, or SEQ ID NO: 6.

6. The seed of embodiment 5, which comprises SEQ ID NO: 3, SEQ ID NO: 4, SEQ ID NO: 5, or SEQ ID NO: 6.

7. A nucleic acid molecule containing a nucleic acid sequence comprising SEQ ID NO: 1, SEQ ID NO: 2, SEQ ID NO: 3, SEQ ID NO: 4, SEQ ID NO: 5 or SEQ ID NO: 6.

8. The nucleic acid molecule of embodiment 7, which comprises SEQ ID NO: 3, SEQ ID NO: 4, SEQ ID NO: 5 or SEQ ID NO: 6.

9. The nucleic acid molecule according to embodiment 7 or 8, wherein the nucleic acid molecule is comprised in a corn seed, an example of said seed deposited at the NCMA under the Accession No. 202310010.

10. An amplicon comprising the nucleic acid molecule of embodiment 7 or 8.

11. A pair of polynucleotide primers comprising a first polynucleotide primer and a second polynucleotide primer which function together in the presence of a corn elite event MZIR260 DNA template in a sample to produce an amplicon diagnostic for the corn elite event MZIR260.

12. The pair of polynucleotide primers according to embodiment 11, wherein the corn elite event MZIR260 DNA template comprises SEQ ID NO:1, SEQ ID NO: 2, SEQ ID NO: 3, SEQ ID NO: 4, SEQ ID NO: 5 or SEQ ID NO: 6, or a complement thereof.

13. The pair of polynucleotide primers according to embodiment 11, wherein the first polynucleotide primer comprises at least 10 contiguous nucleotides as set forth in SEQ ID NO: 8, or the complements thereof.

14. The pair of polynucleotide primers according to embodiment 11, wherein the first polynucleotide primer comprises a nucleotide sequence selected from the group consisting of SEQ ID NO: 11, 17, 20, 28, and 32, or the complements thereof.

15. The pair of polynucleotide primer according to embodiment 11, wherein the second polynucleotide primer comprises at least 10 contiguous nucleotides as set forth in SEQ ID NO: 9, or the complements thereof.

16. The pair of polynucleotide primers according to embodiment 11, wherein the second polynucleotide primer comprises a nucleotide sequence selected from the group consisting of SEQ ID NO: 12, 18, 21, 29, and 33, or the complements thereof.

17. The pair of polynucleotide primers according to embodiment 11 to 14, wherein the second polynucleotide primer comprises at least 10 contiguous nucleotide as set forth in SEQ ID NO: 7, or the complements thereof

18. A method of detecting the presence of a nucleic acid molecule that is unique to elite event MZIR260 in a sample comprising corn nucleic acids, the method comprising: a) providing a genomic DNA sample from a corn plant that comprises a nucleic acid molecule that is unique to elite event MZIR260; b) combining the nucleic acid molecule with a pair of polynucleotide primers of any one of embodiments 11-16; c) performing a nucleic acid amplification reaction which results in an amplicon diagnostic for the corn elite event MZIR260, wherein the amplicon comprises a nucleic acid sequence comprising SEQ ID NO: 1, SEQ ID NO: 2, SEQ ID NO: 3, SEQ ID NO: 4, SEQ ID NO: 5 or SEQ ID NO: 6, or a complement thereof; and d) detecting the amplicon.

19. The method of embodiment 18, wherein the detecting of the amplicon comprises contacting the amplicon with a probe that is complementary to the amplicon.

20. The method of embodiment 18, wherein the detecting of the amplicon comprises sequencing the amplicon.

21. The method of embodiment 18, further comprising selecting a plant comprising the amplicon for the purposes of breeding corn comprising corn elite event MZIR260.

22. A DNA molecule comprising the amplicon produced by the method of embodiment 18.

23. A method of confirming the absence of a nucleic acid molecule that is unique to elite event MZIR260 in a sample comprising corn nucleic acids, the method comprising: a) providing a genomic DNA sample from a corn plant; b) combining the genomic DNA sample with a pair of polynucleotide primers of embodiment 13 or 14 with a pair of polynucleotide primers that is complementary to a corn native genomic sequence as a positive control; c) performing a nucleic acid amplification reaction which results in no amplicon specific to elite event MZIR260 and results in an amplicon specific to the corn native genome positive control; and d) detecting the amplicon specific to the corn native genome positive control.

24. A biological sample derived from the transgenic corn plant of embodiment 3 or the seed of embodiment 5, wherein the sample comprises a nucleotide sequence which comprises or is complementary to any one of SEQ ID NOs: 1-6.

25. The biological sample of embodiment 18, wherein the sample is derived from a corn commodity product.

26. An extract derived from the biological sample of embodiment 25, said extract comprising a nucleotide sequence which comprises or is complementary to any one of SEQ ID NOs: 1-6.

27. A method of producing a corn commodity product, comprising the steps of: a) obtaining the transgenic corn plant of embodiment 2 or 3; and b) producing a corn commodity product from the said transgenic corn plant, cells, or tissue thereof.

28. A corn commodity product comprising a DNA molecule unique for elite event MZIR260, wherein said DNA molecule comprises any one of SEQ ID NOs: 1-6.

29. A non-living plant material comprising the nucleic acid molecule of embodiment 7.

30. A method of producing hybrid corn seed comprising corn elite event MZIR260, comprising: a) crossing the transgenic corn plant of embodiment 2 with a second corn plant that is different from the transgenic corn plan of embodiment 2 to produce hybrid corn seed; and b) harvesting the hybrid corn seeds produced.

31. Hybrid corn seed produced by the method of embodiment 30.

32. Hybrid corn plants produced by growing hybrid corn seed of embodiment 31.

33. A kit for detecting the presence of corn elite event MZIR260 nucleic acids in a biological sample, the kit comprising at least one DNA molecule comprising a sufficient length of contiguous nucleotides that comprises or is complementary to any of SEQ ID NOs: 1-6 that functions as a DNA primer or probe specific for corn elite event MZIR260.

34. Use of the transgenic corn plant of embodiment 2 to produce a corn elite event MZIR260 plant.

35. A method for protecting a corn elite event MZIR260 plant against feeding damage by one or more pests, the method comprising: (a) providing a seed of embodiment 1 or a transgenic corn plant of embodiment 2; and (b) treating the seed or transgenic corn plant with an insecticide.

36. The seed of embodiment 1 treated with an insecticide.

37. The transgenic plant of embodiment 2 treated with an insecticide.

38. A method for protecting a corn plant from insect infestation, wherein said method comprises providing in the diet of a Lepidopteran insect pest an insecticidally effective amount of a transgenic corn plant of embodiment 2.

39. The use of the transgenic corn plant of embodiment 2 for controlling Lepidopteran insect pests.

40. The method of embodiment 38 or use of embodiment 39, wherein the Lepidopteran insect pest is Spodoptera frugiperda (fall armyworm), Spodoptera litura (common cutworm), or Diatraea saccharalis (sugarcane borer).

41. A process for producing corn elite event MZIR260 seed, said process comprising crossing the transgenic corn plant of embodiment 2 with a second corn plant.

42. A process of introducing an additional trait into a corn elite event MZIR260 plant, comprising: (a) crossing a transgenic corn plant of embodiment 2, with another maize plant that comprises an additional trait to produce hybrid progeny plants, (b) selecting hybrid progeny plants that have the additional trait to produce selected hybrid progeny plants; (c) crossing the selected progeny plants with the corn elite event MZIR260 parental plants to produce backcross progeny plants; (d) selecting for backcross progeny plants that have the additional trait to produce selected backcross progeny plants; and (e) repeating steps (c) and (d) at least two or more times to produce backcross progeny plants that comprise the additional trait and corn elite event MZIR260.

43. A plant produced by the process of embodiment 42.

44. A method of making a transgenic maize plant comprising inserting a heterologous nucleic acid at a position on chromosome 2 corresponding to nucleotide coordinates 140849156 and 140851379 in the reference B 73 corn genome.

45. A method of modifying elite event MZIR260, comprising introducing a modification into elite event MZIR260 in a transgenic host cell or a transgenic plant, thereby producing a modified transgenic host cell or a modified transgenic plant, wherein an example of seed comprising said elite event MZIR260 is deposited as NCMA Accession No. 202310010.

46. The method of embodiment 45, wherein the modification is a deletion, an insertion, a substitution, a duplication, or inversion or a combination thereof.

47. The method of embodiment 46, wherein the modification comprises deletion of a portion or all of a PMI coding sequence present in the nucleic acid molecule.

48. The method of embodiment 46, wherein the modification comprises an insertion of a CRISPR-Cas nuclease PAM site within any one or more of SEQ ID NOs: 1-6.

49. The method of any one of embodiments 45 to 48, wherein the modification is introduced using a nuclease or homologous recombination, or a combination thereof.

50. The method of embodiment 49, wherein the nuclease is a CRISPR-Cas nuclease.

51. The method of any one of embodiments 45-48, wherein the method further comprises producing a plant from the modified transgenic host cell and selfing or crossing the plant with another plant, thereby producing a modified transgenic progeny plant.

52. The method of any one of embodiments 45-48, wherein the method further comprises selfing or crossing the modified transgenic plant with another plant, thereby producing a modified transgenic progeny plant.

53. The method of embodiment 51, wherein the method further comprises selfing or outcrossing the modified transgenic progeny plant for at least one additional generation.

54. The method of embodiment 52, wherein the method further comprises selfing or outcrossing the modified transgenic progeny plant for at least one additional generation.

### DESCRIPTION OF THE SEQUENCES IN THE SEQUENCE LISTING

SEQ ID NO: 1 is a nucleic acid sequence of the 5′ genome-insert junction, unique to event MZIR260.
SEQ ID NO: 2 is a nucleic acid sequence of the 3′ genome-insert junction, unique to event MZIR260.
SEQ ID NO: 3 is a nucleic acid sequence of the 5′ genome-insert junction, sequence, plus additional 5′ flanking genomic sequence.
SEQ ID NO: 4 is a nucleic acid sequence of the 3′ genome-insert junction, sequence, plus additional 3′ flanking genomic sequence.
SEQ ID NO: 5 is the nucleic acid sequence of the transgenic cassette insertion, unique to event MZIR260.
SEQ ID NO: 6 is a nucleic acid sequence of the transgenic cassette insertion of SEQ ID NO: 5, plus additional 5′ and 3′ flanking genomic sequences.
SEQ ID NO: 7 is a nucleic acid sequence of the expression cassette comprising the elements listed in Table 1.
SEQ ID NO: 8 is the nucleic acid sequence of the 5′ genomic sequence (1000 bp) flanking the MZIR260 transgenic cassette insertion.
SEQ ID NO: 9 is the nucleic acid sequence of the 3′ genomic sequence (1000 bp) flanking the MZIR260 transgenic cassette insertion.
SEQ ID NO: 10 is the nucleic acid sequence of the amplicon for real-time event-specific MZIR260 method.
SEQ ID NO: 11-13 are primer and probe sequences useful for detection of the MZIR260 event, and which correspond to the amplicon of SEQ ID NO: 10.
SEQ ID NO: 14 is the nucleic acid sequence of a forward primer useful in the detection of eCry1Gb.1Ig-03 in MZIR260.
SEQ ID NO: 15 is the nucleic acid sequence of a reverse primer useful in the detection of eCry1Gb.1Ig-03 in MZIR260.
SEQ ID NO: 16 is the nucleic acid sequence of a probe useful in the detection of eCry1Gb.1Ig-03 in MZIR260.
SEQ ID NO: 17 is the nucleic acid sequence of a forward primer useful in the detection of MZIR260 in the 5′ genome-insertion junction in MZIR260.
SEQ ID NO: 18 is the nucleic acid sequence of a reverse primer useful in the detection of MZIR260 in the 5′ genome-insertion junction in MZIR260.
SEQ ID NO: 19 is the nucleic acid sequence of a probe useful in the detection of MZIR260 in the 5′ genome-insertion junction in MZIR260.
SEQ ID NO: 20 is the nucleic acid sequence of a forward primer useful in the detection of MZIR260 in the 3′ genome-insertion junction in MZIR260.
SEQ ID NO: 21 is the nucleic acid sequence of a reverse primer useful in the detection of MZIR260 in the 3′ genome-insertion junction in MZIR260.
SEQ ID NO: 22 is the nucleic acid sequence of a forward primer useful in the detection of pmi-15 in MZIR260.
SEQ ID NO: 23 is the nucleic acid sequence of a reverse primer useful in the detection of pmi-15 in MZIR260.
SEQ ID NO: 24 is the nucleic acid sequence of a probe useful in the detection of pmi-15 in MZIR260.
SEQ ID NO: 25 is the nucleic acid sequence of a forward primer useful in the detection of adh 1.
SEQ ID NO: 26 is the nucleic acid sequence of a reverse primer useful in the detection of adh 1.
SEQ ID NO: 27 is the nucleic acid sequence of a probe useful in the detection of adh 1.
SEQ ID NO: 28 is the nucleic acid sequence of a forward primer useful in the detection of MZIR260 in the 5′ genome-insertion junction in MZIR260.
SEQ ID NO: 29 is the nucleic acid sequence of a reverse primer useful in the detection of MZIR260 in the 5′ genome-insertion junction in MZIR260.
SEQ ID NO: 30 is the nucleic acid sequence of a forward primer useful in the detection of eCry1Gb.1Ig-03 in MZIR260.
SEQ ID NO: 31 is the nucleic acid sequence of a reverse primer useful in the detection of eCry1Gb.1Ig-03 in MZIR260.
SEQ ID NO: 32 is the nucleic acid sequence of a forward primer useful in the detection of MZIR260 in the 3′ genome-insertion junction in MZIR260.
SEQ ID NO: 33 is the nucleic acid sequence of a reverse primer useful in the detection of MZIR260 in the 3′ genome-insertion junction in MZIR260.
SEQ ID NO: 34 is the nucleic acid sequence of the forward primer used for detection of the homopolymer region in MZIR260.
SEQ ID NO: 35 is the nucleic acid sequence of the reverse primer used for detection of the homopolymer region in MZIR260.
SEQ ID NO: 36-51 are nucleic acid sequences of primers used for genome insertion site characterization of MZIR260.
SEQ ID NO: 52-60 are nucleic acid sequences of primers used for chromosomal location of the MZIR260 insertion.
SEQ ID NO: 61 and 62 are the ribonucleic acid sequences used for characterization of the homopolymer region of MZIR260.
SEQ ID NO: 63-68 are nucleic acid sequences of the primers used for characterization of the MZIR260 insertion.
SEQ ID NO: 69 is the nucleic acid sequence of the Ubi4-02 promoter.
SEQ ID NO: 70 is the nucleic acid sequence of the Ubi1-43 promoter.
SEQ ID NO: 71 is the nucleic acid sequence of the coding sequence of eCry1Gb.1Ig-03.
SEQ ID NO: 72 is the nucleic acid sequence of the coding sequence of pmi-15.
SEQ ID NO: 73 is the nucleic acid sequence of the Ubi361-05 terminator.
SEQ ID NO: 74 is the nucleic acid sequence of the Ubi1-04 terminator.
SEQ ID NO: 75 is the amino acid sequence of eCry1Gb.1Ig.
SEQ ID NO: 76 is the amino acid sequence of PMI-15.
SEQ ID NO: 77 is the nucleic acid sequence of the maize genomic site where the MZIR260 transformation cassette was inserted.
SEQ ID NO: 78 is the nucleic acid sequence of the 5′ genome-insert junction (corresponding to positions 1-208 of SEQ ID NO: 1).
SEQ ID NO: 79 is the nucleic acid sequence of the 3′ genome-insert junction (corresponding to positions 218-351 of SEQ ID NO: 2)

### BRIEF DESCRIPTION OF DRAWINGS

FIG. 1 is a graphical map illustrating the organization of the elements comprising the heterologous nucleic acid sequences inserted into the genome of corn to create elite event MZIR260.

FIG. 2 is a graphical map illustrating the general locations of the real-time qPCR primers and probes in the T-DNA of the transformation 24795 plasmid.

FIG. 3 represents the region of the B 73 maize chromosome 2 with the highest similarity to MZIR260 genomic insertion site. The inset depicts the location on chromosome 2 of the MZIR260 transgenic locus.

### DETAILED DESCRIPTION

The foregoing and other aspects of the invention will become more apparent from the following detailed description.

The following definitions and methods are provided to better define the invention and to guide those of ordinary skill in the art in the practice of the invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1994.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

A “biological sample” is a plant, plant material or products comprising plant material.

The term “plant” is intended to encompass corn (Zea mays) plant tissues, at any stage of maturity, as well as cells, tissues, organs taken from or derived from any such plant, including without limitation, any seeds, leaves, stems, flowers, roots, single cells, gametes, cell cultures, tissue cultures or protoplasts.

“Plant material”, as used herein refers to material which is obtained or derived from a plant. Products comprising plant material relate to food, feed or other products which are produced using plant material or can be contaminated by plant material. A biological sample may be crushed, non-viable material. A biological sample may be derived from a commodity product, such as a corn commodity product. Corn, also known as maize, is used as human food, livestock feed, and as raw material in industry. The food uses of maize, in addition to human consumption of maize kernels, include both products of dry- and wet-milling industries. The principal products of maize dry milling are grits, meal and flour. The maize wet-milling industry can provide maize starch, maize syrups and dextrose for food use. Maize oil is recovered from maize germ, which is a by-product of both dry- and wet-milling industries.

A corn commodity product is typically derived from the grain, from the ear of the corn. Corn commodity products may also be derived from non-grain parts of the corn plant. A number of different industrial processes can be employed to extract or utilize these plant products, as are well known in the art. Corn commodity products include corn flour, corn meal, corn syrup, corn oil, corn starch, and cereals manufactured in whole or in part to contain corn by-products. Corn commodity products may be crushed, non-viable material derived from corn seeds but which are no longer capable of germination. Corn, including both grain and non-grain portions of the plant, is also used extensively as livestock feed, primarily for beef cattle, dairy cattle, hogs, and poultry. Industrial uses of maize include production of ethanol, maize starch in the wet-milling industry and maize flour in the dry-milling industry. The industrial applications of maize starch and flour are based on functional properties, such as viscosity, film formation, adhesive properties and ability to suspend particles. The maize starch and flour have application in the paper and textile industries. Other industrial uses include applications in adhesives, building materials, foundry binders, laundry starches, explosives, oil-well muds, and other mining applications. Plant parts other than the grain of maize are also used in industry: for example, stalks and husks are made into paper and wallboard and cobs are used for fuel and to make charcoal.

A biological extract or “extract” may be derived from a biological sample or from a corn commodity product. A biological extract may be crushed or otherwise extracted from biological material, and is no longer viable or capable of germination. It is understood that, in the context of the invention, such biological samples or extracts are tested for the presence or absence of nucleic acids specific to corn elite event MZIR260, implying the presence or absence of nucleic acids in the samples. Thus, the methods referred to herein for identifying elite event MZIR260 in biological samples or extracts relate to the identification in biological samples or extracts of nucleic acids which are from elite event MZIR260 and are diagnostic for elite event MZIR260.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

A “control” plant can include a positive control in which the corn plant contains the MZIR260 event, or a negative control in which the corn plant does not contain the MZIR260 event (e.g., either contains an event other than MZIR260 or an unmodified corn plant).

“Detection kit” as used herein refers to a kit used to detect the presence or absence of DNA from corn elite event MZIR260 plants in a sample comprising nucleic acid probes and primers of the invention, which hybridize specifically under high stringency conditions to a target DNA sequence, and other materials necessary to enable nucleic acid hybridization or amplification methods.

“Expression cassette” as used herein means a nucleic acid sequence capable of directing expression of a particular nucleotide sequence(s) in an appropriate host cell, comprising one or more transgenes, each transgene comprising a promoter operably linked to a nucleotide sequence of interest which is operably linked to termination signals. Each transgene also typically comprises sequences required for proper translation of the nucleotide sequence. The expression cassette comprising the nucleotide sequence(s) of interest may have at least one of its components heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event.

A “gene” comprises a coding nucleic acid sequence and typically also comprises other, primarily regulatory, nucleic acids responsible for the control of the expression, that is to say the transcription and translation, of the coding portion. A gene may also comprise other 5′ and 3′ untranslated sequences and termination sequences. Further elements that may be present are, for example, introns. The regulatory nucleic acid sequence of the gene may not normally be operatively linked to the associated nucleic acid sequence as found in nature and thus would be a chimeric gene.

The term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, which can be cultured into a whole plant.

The term “heterologous” when used in reference to a gene or a polynucleotide or a polypeptide refers to a gene or a polynucleotide or a polypeptide that is or contains a part thereof not in its natural environment (i.e., has been altered by the hand of man). For example, a heterologous gene may include a polynucleotide from one species introduced into another species. A heterologous gene may also include a polynucleotide native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the disclosure, heterologous genes are distinguished from endogenous plant genes in that the heterologous gene polynucleotides are typically joined to polynucleotides comprising regulatory elements such as promoters that are not found naturally associated with the gene for the protein encoded by the heterologous gene or with plant gene polynucleotides in the chromosome, or are associated with portions of the chromosome not found in nature (e.g., genes expressed in loci where the gene is not normally expressed). Further, a “heterologous” polynucleotide refers to a polynucleotide not naturally associated with a host cell into which it is introduced, including non-naturally occurring multiple copies of a naturally occurring polynucleotide.

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM 62 for protein with default parameters (Gap Open=10, Gap Extend=0.5, End Gap Penalty=False, End Gap Open=10, End Gap Extend=0.5) or DNA full for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/ and as described in the following publication: “The EMBL-EBI search and sequence analysis tools APIs in 2019.” Madeira et al. Nucleic Acids Research, June 2019, 47 (W 1): W 636-W 641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

“Insecticidal” is defined as a toxic biological activity capable of controlling insects, preferably by killing them. To “control” insects means to inhibit, through a toxic effect, the ability of insect pests to survive, grow, feed, and/or reproduce, and/or to limit insect-related damage or loss in crop plants and/or to protect the yield potential of a crop when grown in the presence of insect pests. To “control” insects may or may not mean killing the insects, although in some embodiments of the disclosure, “control” of the insect means killing the insects. A transgenic plant with “enhanced insecticidal properties” is a plant that is expresses a protein or proteins at effective insect-controlling amounts, so that, in some embodiments, the plant is insecticidal to an increased range of insect species, relative to a plant of the same kind which is not transformed. This increased range of insect species includes insect plant pests, such as lepidopteran insect pests, e.g., Spodoptera frugiperda (fall armyworm).

“Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

“MZIR260-specific” or “diagnostic of elite event MZIR260” refers to a nucleotide sequence which is suitable for discriminatively identifying elite event MZIR260 in plants, plant material, biological samples, extracts or in products such as, but not limited to, corn plants and food or feed products (fresh or processed) comprising or derived from plant material.

“Insert DNA” or “insert sequence” refers to the heterologous DNA present in transformed plant material, and includes expression cassettes present in the transformation plasmid.

Insert DNA is derived from a T-DNA, which is contained within a binary vector used in Agrobacterium-mediated transformation of a plant. “Flanking DNA” or “flanking sequence” can exist of either genomic DNA naturally present in an organism such as a plant, or foreign (heterologous) DNA introduced via the transformation process, which is extraneous to the original insert DNA molecule, e.g. fragments associated with the transformation event. Flanking sequence as used herein refers to a sequence of at least 10 bp, at least 20 bp, at least 50 bp, at least 100 bp, at least 150 bp, at least 200 bp, at least 250 bp, at least 300 bp, at least 400 bp, at least 500 bp, at least 1000 bp, at least 2000 bp, at least 3000 bp, at least 4000 bp, and at least 5000 bp or of up to 10 bp, up to 20 bp, up to 50 bp, up to 100 bp, up to 150 bp, up to 200 bp, up to 250 bp, up to 300 bp, up to 400 bp, up to 500 bp, up to 1000 bp, up to 2000 bp, up to 3000 bp, up to 4000 bp, and up to 5000 bp, which is located either immediately upstream of and contiguous with or immediately downstream of and contiguous with the original foreign insert DNA molecule. Transformation procedures leading to random integration of the foreign DNA will result in transformants containing different flanking regions characteristic of and likely unique to each transformant. When recombinant DNA is introduced into a plant through traditional crossing, its immediate flanking sequences will generally not be changed. Transformants will also contain unique junctions between a piece of heterologous insert DNA and genomic DNA, or two pieces of genomic DNA, or two pieces of heterologous DNA. A “junction” is a point where two specific DNA fragments join. For example, in the case of MZIR260 a junction exists where the insert DNA joins genomic flanking DNA. A junction point also exists in a transformed organism where two DNA fragments join together in a manner that is modified from that found in the native organism. “Junction DNA” or “junction sequence” refers to DNA that comprises a junction point. Two junction sequences set forth in this disclosure are the junction point between the maize genomic DNA and the 5′ end of the insert as set forth in SEQ ID NO: 1, and the junction point between the 3′ end of the insert and maize genomic DNA as set forth in SEQ ID NO: 2. An event may be defined or identifiable by its junction sequences. These junction sequences can be transmitted to progeny and introgressed into other germplasms via traditional crossing.

The term “event” refers to a artificial genetic locus that, as a result of transformation, carries a foreign inserted DNA that includes an expression cassette for at least one gene of interest and also comprises flanking genomic sequence immediately adjacent to the inserted DNA that would be expected to be transferred to a progeny that receives the inserted DNA as a result of a sexual cross of one parental line which comprises the inserted DNA (e.g., the original transformant and progeny resulting from selfing) and a parental line that does not contain the inserted DNA. The event comprises the junction sequences and the foreign inserted DNA. The presence of an event in a cell may be identified genotypically by one or both of its junction sequences, or any other sequence present in the inserted DNA that is unique to the event. At the genetic level, an event is part of the genome of a plant.

The term, for example, “event MZIR260 plant”, “MZIR260 plant”, “elite event MZIR260 plant”, or “event MZIR260 corn” refers to a corn plant that comprises the MZIR260 event. An event MZIR260 plant may refer to progeny of the original transformant. The term “event MZIR260 plant” also refers to progeny produced by a sexual outcross between an event MZIR260 plant and another corn line. Even after repeated backcrossing to a recurrent parent, the inserted DNA, genomic flanking DNA, and junction sequences from the originally transformed plant are present in the progeny of the cross at the same chromosomal location. Similarly, an “MZIR260 seed” refers to a seed which comprises the MZIR260 event.

An “elite event” comprises all of the desirable characteristics of an event required for commercial utility. An elite event comprises one and only one complete copy of the transgenic cassette, with an absence of vector backbone sequence. The transgenic cassette is inserted at a desirable location in the genome, which, e.g., may allow for easy introgression into desired commercial genetic backgrounds. The genomic location of the transgenic cassette of an elite event also allows for proper expression of the trait protein(s) present in the transgenic cassette. The expression of the trait protein(s) of the transgenic cassette in an elite event is correct, appropriate, and stable spatially and temporally, both in heterozygous (or hemizygous) and homozygous conditions; is at a commercially acceptable level for a range of environmental conditions in which the plants carrying the event are likely to be exposed in normal agronomic use; and is stable through multiple generations of progeny. The transgenic cassette of the elite event also shows normal Mendelian segregation. An elite event has desirable agronomic characteristics, such as yield, vigor, fertility, and the like, which are not significantly negatively impacted by the presence of the event in the genome of the plant. An elite event generally has a superior combination of efficacy and agronomic performance in broad genotype backgrounds and across multiple environmental locations. An “elite event” may refer to a genetic locus comprising a foreign DNA as a transgenic cassette, which meets the above-described criteria. A plant, plant material or progeny such as seeds can comprise one or more elite events in its genome. The likelihood of having all of these characteristics in an event is small, such that an elite event is non-obvious and atypical of events recovered from the transformation process.

Plants harboring elite event MZIR260 are characterized by their insect resistance, particularly to Spodoptera spp or Diatraea spp. Corn plants comprising elite event MZIR260 are useful in controlling Spodoptera frugiperda (fall armyworm, FAW) and, optionally, Diatraea saccharalis (sugarcane borer, SCB) and/or Spodoptera litura (common cutworm, CCW). Plants harboring elite event MZIR260 are also characterized by having agronomic characteristics that are comparable to commercially available varieties of corn, in the absence of insect pest pressure. These characteristics make the elite event MZIR260 very useful for control of Spodoptera spp. insect pests of corn, particularly Spodoptera frugiperda (fall armyworm, FAW) and Spodoptera litura (common cutworm, CCW), as well as control of Diatraea spp. insects, particularly Diatraea saccharalis (sugarcane borer, SCB), in corn fields. Because eCry1Gb.1Ig also controls species of fall armyworm that are resistant to other Cry toxins, such as Cry1F, elite event MZIR260 also can provide effective control of pests that have developed field resistance to other commercially-available modalities used for FAW and/or SCB control.

A “heterologous” nucleic acid sequence is a nucleic acid sequence not naturally associated with a host cell into which it is introduced, including non-naturally occurring multiple copies of a naturally occurring nucleic acid sequence.

A “homologous” nucleic acid sequence is a nucleic acid sequence naturally associated with a host cell into which it is introduced.

The term “isolated” when used in relation to a nucleic acid refers to a nucleic acid sequence that is identified and separated from at least one contaminant nucleic acid with which it is ordinarily associated in its natural source. An isolated nucleic acid is present in a form or setting that is different from that in which it is found in nature. In contrast, a non-isolated nucleic acids such as DNA and RNA found in the state they exist in nature. An isolated nucleic acid may be in a transgenic plant or biological sample and still be considered “isolated”.

“Operably-linked” refers to the association of nucleic acid sequences on a single nucleic acid fragment so that the function of one affects the function of the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

The tools developed to identify an elite event or the plant or plant material comprising an elite event, or products which comprise plant material comprising the elite event, are based on the specific genomic characteristics of the elite event, such as, a specific restriction map of the genomic region comprising the inserted DNA, molecular markers, or the sequence of the flanking region(s) of the inserted DNA.

Once one or both of the flanking regions of the inserted DNA, or transgenic cassette, have been sequenced, primers and probes can be developed which specifically recognize this (these) sequence(s) in the nucleic acid (DNA or RNA) of a sample by way of a molecular biological technique. For instance, a PCR method can be developed to identify the elite event in biological samples (such as samples of plants, plant material or products comprising plant material). Such a PCR is based on at least two specific “primers”, e.g., one recognizing a sequence within the 5′ or 3′ flanking region of the elite event and the other recognizing a sequence within the foreign DNA. The primers preferably have a sequence of between 15 and 35 nucleotides which under optimized PCR conditions “specifically recognize” a sequence within the 5′ or 3′ flanking region of the elite event and the foreign DNA of the elite event respectively, so that a specific fragment (“integration fragment” or discriminating amplicon) is amplified from a nucleic acid sample comprising the elite event. This means that only the targeted integration fragment, and no other sequence in the plant genome or foreign DNA, is amplified under optimized PCR conditions.

“Primers” as used herein are isolated nucleic acids that are annealed to a complementary target DNA strand by nucleic acid hybridization to form a hybrid between the primer and the target DNA strand, then extended along the target DNA strand by a polymerase, such as DNA polymerase. A primer pair comprises a “forward” primer and a “reverse” primer. Primer pairs or sets can be used for amplification of a nucleic acid molecule, for example, by the polymerase chain reaction (PCR) or other conventional nucleic-acid amplification methods.

PCR primers suitable for identification of elite corn event MZIR260 may be the following:
a) oligonucleotides ranging in length from 17 bp to about 200 bp, comprising a nucleotide sequence of at least 17 consecutive nucleotides, preferably 20 consecutive nucleotides, selected from DNA in the 5′ flanking sequence (SEQ ID NO: 8), such that the primer recognizes the 5′ flanking sequence; or
b) oligonucleotides ranging in length from 17 bp to about 200 bp, comprising a nucleotide sequence of at least 17 consecutive nucleotides, preferably 20 consecutive nucleotides, selected from DNA in the 3′ flanking sequence (SEQ ID NO: 9), such that the primer recognizes the 3′ flanking sequence; or
c) oligonucleotides ranging in length from 17 bp to about 200 bp, comprising a nucleotide sequence of at least 17 consecutive nucleotides, preferably 20 consecutive nucleotides, selected from the DNA in the transgenic cassette insertion (SEQ ID NO: 5), such that the primer recognizes the transgenic cassette.

The primers may of course be longer than the mentioned 17 consecutive nucleotides, and may, e.g., be 20, 21, 22, 23, 24, 25, 30, 35, 50, 75, 100, 150, 200 bp long or even longer. The primers may entirely consist of nucleotide sequence selected from the mentioned nucleotide sequences of flanking sequences and transgene DNA sequences. However, the nucleotide sequence of the primers at their 5′ end (i.e. outside of the 3′-located 17 consecutive nucleotides) is less critical. Thus, the 5′ sequence of the primers may comprise or consist of a nucleotide sequence selected from the flanking sequences or foreign DNA, as appropriate, but may contain several (e.g., 1, 2, 5, or 10) mismatches. The 5′ sequence of the primers may even entirely be a nucleotide sequence unrelated to the flanking sequences or foreign DNA, such as, e.g., a nucleotide sequence representing one or more restriction enzyme recognition sites. Such unrelated sequences or flanking DNA sequences with mismatches should preferably be not longer than 100, more preferably not longer than 50 or 25 nucleotides.

Moreover, suitable primers may comprise, consist or consist essentially of a nucleotide sequence spanning the junction region between the plant DNA derived sequences and the inserted DNA sequences (SEQ ID NO: 1 and 2). It will also be immediately clear to the skilled artisan that properly selected PCR primer pairs should also not comprise sequences complementary to each other.

Examples of suitable primers for detection or identification of elite event MZIR260 include SEQ ID NOs: 11, 12, 17, 18, 20, 21, 28, 29, 32, and 33, and complements thereof. Other examples of suitable oligonucleotide primers for the detection or identification of elite event MZIR260 comprise at least 10 contiguous nucleotides of SEQ ID NO: 6, 8, and 9, and complements thereof. A person of ordinary skill in the art will appreciate that for a primer set to be diagnostic for corn elite event MZIR260, the resulting amplicon ideally comprises SEQ ID NO: 1 and/or SEQ ID NO: 2 (for example, the amplicon set forth in SEQ ID NO: 10).

A “probe” is an isolated nucleic acid to which is attached a detectable label or reporter molecule, such as a radioactive isotope, ligand, chemiluminescent agent, fluorescent agent, or enzyme. Such a probe is complimentary to a strand of a target nucleic acid, in the case of the invention, to a strand of genomic DNA from corn elite event MZIR260. The genomic DNA of elite event MZIR260 can be from a corn plant or from a sample that includes DNA from the event. Probes according to the invention include not only deoxyribonucleic or ribonucleic acids but also polyamides and other probe materials that bind specifically to a target DNA sequence and can be used to detect the presence of that target DNA sequence.

Probes and primers are generally 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 polynucleotides or more in length. Such primers and probes hybridize specifically to a target sequence under high stringency hybridization conditions. Primers and probes according to the invention may have complete sequence complementarity with the target sequence, although probes differing from the target sequence and which retain the ability to hybridize to target sequences may be designed by conventional methods. Probe sequences useful for detection or identification of elite event MZIR260 include SEQ ID NOs: 13 and 19, and complements thereof

“Stringent conditions” or “stringent hybridization conditions” include reference to conditions under which a probe will hybridize to its target sequence, to a detectably greater degree than to other sequences. Stringent conditions are target-sequence-dependent and will differ depending on the structure of the polynucleotide. By controlling the stringency of the hybridization and/or wash conditions, target sequences can be identified which are 100% complementary to the probe (homologous probing). Alternatively, stringency conditions can be adjusted to allow some mismatching in sequences so that lower degrees of similarity are detected (heterologous probing). Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, Part I, Chapter 2 “Overview of principles of hybridization and the strategy of nucleic acid probe assays”, Elsevier: New York; and Current Protocols in Molecular Biology, Chapter 2, Ausubel et al., Eds., Greene Publishing and Wiley-Interscience: New York (1995), and also Sambrook et al. (2001) Molecular Cloning: A Laboratory Manual (5th Ed. Cols Spring Harbor Laboratory, Cold Spring Harbor, NY).

Specificity is typically the function of post-hybridization washes, the critical factors being the ionic strength and temperature of the final wash solution. Generally, high stringency hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, under high stringency conditions a probe will hybridize to its target subsequence, but to no other sequences.

An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.15M NaCl at 72° C. for about 15 minutes. An example of high stringency wash conditions is a 0.2×SSC wash at 65° C. for 15 minutes (see, Sambrook, infra, for a description of SSC buffer).

Exemplary hybridization conditions for the invention include hybridization in 7% SDS, 0.25 M NaPO₄ PH 7.2 at 67° C. overnight, followed by two washings in 5% SDS, 0.20 M NaPO₄ pH 7.2 at 65° C. for 30 minutes each wash, and two washings in 1% SDS, 0.20 M NaPO₄ pH 7.2 at 65° C. for 30 minutes each wash. An exemplary medium stringency wash for a duplex of, e.g., more than 100 nucleotides, is 1×SSC at 45° C. for 15 minutes. An exemplary low stringency wash for a duplex of, e.g., more than 100 nucleotides, is 46×SSC at 40° C. for 15 minutes.

For probes of about 10 to 50 nucleotides, high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, e.g., when a copy of a nucleic acid is created using the maximum codon degeneracy permitted by the genetic code.

The following are exemplary sets of hybridization/wash conditions that may be used to hybridize nucleotide sequences that are substantially identical to reference nucleotide sequences of the invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C. The sequences of the invention may be detected using all the above conditions. For the purposes of defining the invention, the high stringency conditions are used.

As used herein, “amplified DNA” or “amplicon” refers to the product of nucleic acid amplification of a target nucleic acid sequence that is part of a nucleic acid template. For example, to determine whether a corn plant resulting from a sexual cross contains DNA from elite event MZIR260, DNA extracted from the corn plant tissue sample may be subjected to a nucleic acid amplification method using a DNA primer pair that includes a first primer derived from flanking sequence adjacent to the insertion site of inserted heterologous DNA, and a second primer derived from the inserted heterologous DNA to produce an amplicon that is diagnostic for the presence of the event DNA. Alternatively, the second primer may be derived from the flanking sequence. The amplicon is of a length and has a sequence that is also diagnostic for the event. The amplicon may range in length from the combined length of the primer pairs plus one nucleotide base pair to any length of amplicon producible by a DNA amplification protocol, and/or the combined length of the primer pairs plus about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500, 750, 1000, 1250, 1500, 1750, 2000, or more polynucleotides, plus or minus any of the increments listed above. Alternatively, primer pairs can be derived from flanking sequence on both sides of the inserted DNA so as to produce an amplicon that includes the entire insert nucleotide sequence of the transgenes as well as the sequence flanking the transgenic insert. A member of a primer pair derived from the flanking sequence may be located a distance from the inserted DNA sequence, this distance can range from one nucleotide base pair up to the limits of the amplification reaction, or about 20,000 bp. The use of the term “amplicon” specifically excludes primer dimers.

TaqMan (ThermoFisher Scientific, Waltham, MA, USA.) is described as a method of detecting and quantifying the presence of a DNA sequence and is fully understood in the instructions provided by the manufacturer. Briefly, a FRET oligonucleotide probe is designed which overlaps the flanking and insert DNA junction. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. This fluorescent signal can be quantified.

“Transformation” is a process for introducing heterologous nucleic acid into a host cell or organism. In particular embodiments, “transformation” means the stable integration of a DNA molecule into the genome of an organism of interest.

“Transformed/transgenic/recombinant” refer to a host organism such as a bacterium or a plant into which a heterologous nucleic acid molecule has been introduced. The nucleic acid molecule can be stably integrated into the genome of the host. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule. As used herein, “transgenic” refers to a plant, plant cell, or multitude of structured or unstructured plant cells having integrated a nucleic acid representing a gene of interest into the plant genome, and typically into a chromosome of a cell nucleus, mitochondria or other organelle containing chromosomes, at a locus different to, or in a number of copies greater than, that normally present in the native plant or plant cell. Transgenic plants result from the manipulation and insertion of such nucleic acid sequences, as opposed to naturally occurring mutations, to produce a non-naturally occurring plant or a plant with a non-naturally occurring genotype. Techniques for transformation of plants and plant cells are well known in the art and may comprise for example electroporation, microinjection, A grobacterium-mediated transformation, and ballistic transformation.

As used herein, the term “transgene” refers to a DNA molecule artificially incorporated into an organism's genome as a result of human intervention, such as by plant transformation methods. A transgene may be heterologous to the organism.

As used herein, the term “insect resistant” refers to effecting changes in insect feeding, growth, health, and/or behavior at any stage of development, including but not limited to: killing the insect; retarding growth; reducing reproductive capability; inhibiting feeding; and the like.

As used herein, “stacking” is combining desired genes and/or events into one transgenic plant line. As one approach, plant breeders stack events by making crosses between parents that each have a desired event and then identifying offspring that have both of these desired events (so-called “breeding stacks”). Another way to stack genes is by including them in the same event (e.g., two trait genes in one expression cassette) in a transgenic plant line (so-called “molecular stacks”), which can be accomplished by transferring the genes at the same time during transformation or by re-transforming a transgenic plant with another gene of interest.

The present invention relates to corn elite event MZIR260 that provides effective control of lepidopteran pests such as Spodoptera frugiperda (fall armyworm, FAW) or Spodoptera litura (CCW), particularly FAW or CCW pests that have developed resistance to existing trait proteins for controlling this pest, and Diatraea saccharalis (sugarcane borer, SCB). Control of FAW, CCW and SCB is provided by the presence of the eCry1Gb.1Ig gene (SEQ ID NO: 71, which encodes the amino acid sequence of SEQ ID NO: 75), which is a chimera of the Cry1Gb and Cry1Ig genes. The invention is particularly drawn to an elite corn event designated elite event MZIR260, as well as to compositions and methods for use of elite event MZIR260 and for detecting nucleic acids from this event in a biological sample. The invention is further drawn to corn plants comprising elite event MZIR260 or nucleic acid molecules from elite event MZIR260, to transgenic seed from the corn plants, and to methods for producing a corn plant comprising elite event MZIR260 or nucleic acid molecules from elite event MZIR260 by crossing a corn parent plant (e.g., an inbred parent plant) comprising elite event MZIR260 or nucleic acid molecules from elite event MZIR260 with itself or another corn line. Corn plants comprising elite event MZIR260 of the invention are useful for controlling insect pests, particularly Spodoptera spp. and/or Diatraea spp. insects.

The present invention encompasses a novel elite event MZIR260, which is a result of the random insertion of a transgenic cassette in the genome of a corn plant by Agrobacterium-mediates transformation. It is recognized in the art that the genomic location of such an insertion cannot be predicted. The present invention encompasses the particular novel sequences, namely SEQ ID NO: 1, SEQ ID NO: 2, SEQ ID NO: 3, SEQ ID NO: 4, SEQ ID NO: 5, or SEQ ID NO: 6, and complements thereof, which are diagnostic for elite event MZIR260. Furthermore, the present invention encompasses a corn plant comprising elite event MZIR260, an example of seed of such corn plant which is deposited at Bigelow Laboratory for Oceanic Sciences, National Center for Marine Algae and Microbiota as NCMA Accession NO: 202310010. In some embodiments, a corn plant comprising elite event MZIR260 contains within its genome SEQ ID NO: 1, SEQ ID NO: 2, and SEQ ID NO: 5. Further, the present invention encompasses specific tools in the form of specific polynucleotide molecules that are capable of identifying elite event MZIR260.

In one embodiment, the invention encompasses a seed of a transgenic corn plant comprising elite event MZIR260. An example of said seed has been deposited as NCMA Accession No: 202310010. The transgenic seed, a transgenic plant, transgenic cell, and transgenic tissue of elite event MZIR260 comprises a nucleic acid molecule with at least 90%, 91%, 92%, 93%, 94%, 95%, 96%, 97%, 98%, 99%, or 100% identity to SEQ ID NO: 1, SEQ ID NO: 2, SEQ ID NO: 3, SEQ ID NO: 4, SEQ ID NO: 5, and/or SEQ ID NO: 6, and complements thereof. These sequences define a point of insertion of a heterologous DNA sequence inserted into the corn plant genome of corn elite event MZIR260. The invention further comprises a transgenic corn plant (e.g., a whole plant or cell or tissue thereof) comprising elite event MZIR260, e.g., capable of producing an elite event MZIR260 diagnostic amplicon, wherein said diagnostic amplicon comprises or hybridizes under stringent conditions to SEQ ID NO: 1, SEQ ID NO: 2, SEQ ID NO: 3, or SEQ ID NO: 4. The invention further comprises a transgenic insect resistant plant (e.g., a whole plant or cell or tissue thereof) comprising: a DNA construct comprising two expression cassettes, wherein said first expression cassette in operable linkage comprises: (i) a Ubi4-02 promoter represented by SEQ ID NO: 69; (ii) an engineered insecticidal protein eCry1Gb.1Ig coding sequence represented by SEQ ID NO: 71; and (iii) a Ubi361-05 transcriptional terminator represented by SEQ ID NO: 73; and wherein said second expression cassette in operable linkage comprises (i) a Ubi1-43 promoter represented by SEQ ID NO: 70; (ii) a pmi-15 coding sequence represented by SEQ ID NO: 72; and (iii) a Ubi1-04 transcriptional terminator represented by SEQ ID NO: 74, wherein the sequence overlapping the junction between the corn genomic DNA and the 5′ flank of the construct comprises SEQ ID NO: 1 and the overlapping junction between the corn genomic DNA and the 3′ flank of the construct comprises SEQ ID NO: 2, and wherein the DNA construct is present in the corn elite event MZIR260 deposited with Bigelow Laboratory for Ocean Sciences, National Center for Marine Algae and Microbiota (NCMA) Accession No. 202310010. Table 1 describes each of the genetic elements present in the insert of elite event MZIR260.

TABLE 1
Description of Genetic Elements

| | | SE Q | |
|---|---|---|---|
| | | ID | |
| Element | Name | NO | Description with relevant references |
| promoter | SoUbi4-02 | 69 | Constitutive Saccharum officinarum Ubiquitin 4 |
| | | | promoter containing the first intron (NCBI accession |
| | | | number AF093504.1).⁺ |
| promoter | Ubi1-43 | 70 | Promoter region from Zea mays polyubiquitin gene |
| | | | containing the first intron (NCBI accession number |
| | | | S94464.1). Provides constitutive expression in |
| | | | monocots⁺ |
| coding | eCry1Gb.1lg-03 | 71 | Sequence encoding the engineered protein |
| sequence | | | eCry1Gb.1lg, which is a chimera of Cry1Gb and |
| | | | Cry1lg. Silent mutations were introduced to optimize |
| | | | codon usage |
| Coding | PMI-15 | 72 | Escherichia coli gene pmi encoding the enzyme |
| sequence | | | phosphomannose isomerase (PMI) (NCBI accession |
| | | | number M15380.1); this gene is also known as manA. |
| | | | Catalyzes the isomerization of mannose-6-phosphate to |
| | | | fructose-6-phosphate (Negrotto et al. 2000). |
| Terminator | ZmUbi361-05 | 73 | Terminator derived from the maize Ubiquitin gene |
| | | | (Nuccio 2018) terminator. |
| Terminator | Ubi1-04 | 74 | Terminator from the ubiquitin 1 gene from Z. mays |
| | | | (One bp mutation to remove an internal restriction site |
| | | | compared to the Ubi-01 terminator). |

⁺Cristensen A H, Sharrock R A, Quail P H. 1992. Plant Mol Biol 18: 675-689.

In another embodiment, the invention encompasses a preferably isolated nucleic acid molecule comprising SEQ ID NO: 1, SEQ ID NO: 2, SEQ ID NO: 3, SEQ ID NO: 4, SEQ ID NO: 5, or SEQ ID NO: 6, and complements thereof. In another embodiment, the nucleic acid molecule is comprised in a corn plant. In another embodiment, the invention comprises an isolated nucleic acid molecule comprising SEQ ID NO: 1, SEQ ID NO: 2 or both SEQ ID NO:1 and SEQ ID NO: 2.

In one embodiment, the invention encompasses a nucleic acid molecule, optionally isolated, comprising at least 10 or more (for example 15, 20, 25, or 50) contiguous nucleotides of a heterologous DNA sequence inserted into the corn plant genome of corn elite event MZIR260 and at least 10 or more (for example 15, 20, 25, or 50) contiguous nucleotides of a corn plant genome DNA flanking the point of insertion of a heterologous DNA sequence inserted into the corn plant genome of corn elite event MZIR260. Also included are nucleotide sequences that comprise 10 or more nucleotides of contiguous insert sequence from elite event MZIR260 and at least one nucleotide of flanking DNA from elite event MZIR260 adjacent to the insert sequence. Such nucleotide sequences are diagnostic for elite event MZIR260. Nucleic acid amplification of genomic DNA from the elite event MZIR260 may produce an amplicon comprising such diagnostic nucleotide sequences (namely, SEQ ID NO: 1, SEQ ID NO: 2, SEQ ID NO: 3, SEQ ID NO: 4, SEQ ID NO: 5, SEQ ID NO: 6 and the complements thereof).

In another embodiment, the invention encompasses a nucleic acid molecule, optionally isolated, comprising a nucleotide sequence which comprises at least one junction sequence of elite event MZIR260 selected from the group consisting of SEQ ID NO: 1, SEQ ID NO: 2, SEQ ID NO: 3, SEQ ID NO: 4, and complements thereof. In a further embodiment, the invention comprises a nucleic acid molecule relating to the corn elite event MZIR260, characterized in that it comprises or consists of the sequence of SEQ ID NO: 1 or SEQ ID NO: 3. In another embodiment, the invention comprises a nucleic acid molecule relating to the corn elite event MZIR260, characterized in that it comprises or consists of the sequence of SEQ ID NO: 2 or SEQ ID NO: 4. In another embodiment, the invention comprises the use of SEQ ID NO: 1, SEQ ID NO: 2, SEQ ID NO: 3, SEQ ID NO: 4, SEQ ID NO: 5 and/or SEQ ID NO: 6 to identify corn elite event MZIR260 in a plant or biological sample.

In another embodiment, the invention encompasses a nucleic acid molecule, optionally isolated, comprising a nucleotide sequence selected from the group consisting of SEQ ID NO: 1, SEQ ID NO: 2, SEQ ID NO: 3, SEQ ID NO: 4, and complements thereof. In some embodiments, the nucleic acid molecule is no more than 500, 400, 300, 200 or 100 nucleotides in length.

In another embodiment, the invention encompasses a nucleic acid molecule, optionally isolated, comprising a nucleotide sequence selected from the group consisting of SEQ ID NO: 1, SEQ ID NO: 2, SEQ ID NO: 3, SEQ ID NO: 4, SEQ ID NO: 5, SEQ ID NO: 6, and the complements thereof. In a further embodiment, the invention encompasses the nucleic acid molecule described above wherein the molecule is within the genome of a transgenic organism, for example a transgenic maize plant. The invention also includes a genome comprising said nucleic acid molecule.

In another embodiment, the invention encompasses flanking sequence primers for detecting elite event MZIR260. Such flanking sequence primers comprise an isolated nucleic acid sequence comprising at least 10-15 contiguous nucleotides from SEQ ID NO: 8 (designated herein as the 5′ flanking sequence), SEQ ID NO: 9 (designated herein as the 3′ flanking sequence) or the complements thereof. In one aspect of this embodiment the flanking sequence primers are selected from the group consisting of SEQ ID NO: 36-51, and complements thereof. The flanking sequences can be extended to include additional chromosome 2 sequence useful in detecting sequences associated with the corn elite event MZIR260.

In still another embodiment, the invention encompasses a pair of polynucleotide primers comprising a first polynucleotide primer and a second polynucleotide primer which function together in the presence of a corn elite event MZIR260 DNA template in a sample to produce an amplicon diagnostic for the corn elite event MZIR260. In some aspects of this embodiment, the first primer sequence is or is complementary to a corn plant genomic sequence flanking the point of insertion of a heterologous DNA sequence inserted into the corn plant genome of corn elite event MZIR260, and the second polynucleotide primer sequence is or is complementary to the heterologous DNA sequence inserted into the corn plant genome of the corn elite event MZIR260. In some aspects of this embodiment, the first primer sequence is or is complementary to a junction sequence of corn elite event MZIR260, and the second polynucleotide primer sequence is or is complementary to either the heterologous DNA sequence inserted into the corn plant genome of the corn elite event MZIR260 or to a corn plant genomic sequence flanking the point of insertion of a heterologous DNA sequence inserted into the corn plant genome of corn elite event MZIR260. Another embodiment of the invention is the use of these polynucleotide primers to identify corn elite event MZIR260 in a plant.

In one aspect of this embodiment the first polynucleotide primer comprises at least 10 contiguous nucleotides of SEQ ID NO: 1 or SEQ ID NO: 3, or complements thereof. In a further aspect of this embodiment, the first polynucleotide primer comprises the nucleotide sequence set forth in SEQ ID NO: 11, or complements thereof. In yet another aspect of this embodiment, the second polynucleotide primer comprises at least 10 contiguous nucleotides of SEQ ID NO: 2 or SEQ ID NO: 4, or the complements thereof. In still a further aspect of this embodiment, the second polynucleotide primer comprises the nucleotide sequence set forth in SEQ ID NO: 12, or the complements thereof.

In another aspect of this embodiment, the first polynucleotide primer comprises the nucleotide sequence of SEQ ID NO: 11, 17, 20, 28, or 32, or complements thereof, and the second polynucleotide primer comprises the nucleotide sequence of SEQ ID NO: 12, 18, 21, 29, or 33, or the complement thereof, such that the pair function together in the presence of a corn elite event MZIR260 DNA template in a sample to produce an amplicon diagnostic for the corn elite event MZIR260. In another aspect of this embodiment, the first polynucleotide primer comprises at least 10 contiguous nucleotides of SEQ ID NO: 7, or the complements thereof, and the second polynucleotide primer comprises at least 10 contiguous nucleotides of SEQ ID NO: 9, or complements thereof., such that the pair function together in the presence of a corn elite event MZIR260 DNA template in a sample to produce an amplicon diagnostic for the corn elite event MZIR260. Another embodiment of the invention is the use of these polynucleotide primers to identify corn elite event MZIR260 in a plant.

In another aspect of this embodiment, the first polynucleotide primer comprises SEQ ID NO: 11, and the second polynucleotide primer comprises SEQ ID NO: 12, and the pair function together in the presence of a corn elite event MZIR260 DNA template in a sample to produce an amplicon diagnostic for the corn event MZIR260 which can be detected, e.g., by a probe comprising SEQ ID NO: 13, as described in Example 2.

It is well within the skill in the art to obtain additional sequence further into the genome sequence flanking either end of the inserted heterologous DNA sequences for use as a primer sequence that can be used in such primer pairs for amplifying sequences that are diagnostic for the elite event MZIR260. For the purposes of this disclosure, the phrase “further into the genome sequence flanking either end of the inserted heterologous DNA sequences” refers specifically to a sequential movement away from the ends of the inserted heterologous DNA sequences, the points at which the inserted DNA sequences are adjacent to native genomic DNA sequence, and into the genomic DNA of the particular chromosome into which the heterologous DNA sequences were inserted. Preferably, a primer sequence corresponding to or complementary to a part of the insert sequence should prime the transcriptional extension of a nascent strand of DNA or RNA toward the nearest flanking sequence junction. Consequently, a primer sequence corresponding to or complementary to a part of the genomic flanking sequence should prime the transcriptional extension of a nascent strand of DNA or RNA toward the nearest flanking sequence junction. A primer sequence can be, or can be complementary to, a heterologous DNA sequence inserted into the chromosome of the plant, or a genomic flanking sequence. One skilled in the art would readily recognize the benefit of whether a primer sequence would need to be, or would need to be complementary to, the sequence as set forth within the inserted heterologous DNA sequence or as set forth in SEQ ID NO: 1 or SEQ ID NO: 2 depending upon the nature of the product desired to be obtained through the use of the nested set of primers intended for use in amplifying a particular flanking sequence containing the junction between the genomic DNA sequence and the inserted heterologous DNA sequence. Furthermore, one skilled in the art would be able to design primers for one or more native corn genes and/or other sequences present in the corn genome for the purposes of designing a positive control. One such example is the corn Adh1 gene, where examples of suitable primers for producing an amplicon by nucleic acid amplification are well known in the art (see, for example, U.S. Pat. No. 8,466,346, incorporated by reference herein and SEQ ID NO: 25, 26 and 27).

In another embodiment, the invention encompasses a method of detecting the presence of a nucleic acid molecule that is unique to elite event MZIR260 in a sample comprising corn nucleic acids, the method comprising: a) providing a genomic DNA sample from a corn plant that comprises a nucleic acid molecule that is unique to elite event MZIR260 (e.g., by isolating a nucleic acid molecule from corn); b) combining the nucleic acid molecule with a pair of polynucleotide primers of the invention; c) performing a nucleic acid amplification reaction which results in an amplicon diagnostic for the corn elite event MZIR260; and d) detecting the amplicon. Detection can be accomplished, e.g., using a probe of the invention (e.g., comprising a nucleotide sequence of SEQ ID NO: 1, SEQ ID NO: 2, SEQ ID NO: 10, SEQ ID NO: 13, SEQ ID NO: 19, or a complement thereof).

In some embodiments, the invention encompasses using genotyping-by-sequencing to detect the presence of a nucleic acid molecule that is unique to elite event MZIR260. An example method of genotyping-by-sequencing is disclosed in Elshire et al. (A Robust, Simple Genotyping-by-Sequencing (GBS) Approach for High Diversity Species. PLOS ONE 6 (5): e19379 (2011)) and a method specifically for use in corn is disclosed in Wang et al. (Applications of genotyping-by-sequencing (GBS) in maize genetics and breeding. Sci Rep 10, 16308 (2020)).

In another embodiment, the invention encompasses a method of confirming the absence of a nucleic acid molecule that is unique to elite event MZIR260 in a sample comprising corn nucleic acids, the method comprising: a) providing a genomic DNA sample from a corn plant that comprises a nucleic acid molecule that is unique to elite event MZIR260 (e.g., by isolating genomic DNA from corn); b) combining the nucleic acid molecule with a pair of polynucleotide primers of the invention and with a pair of polynucleotide primers that is complementary to a corn native genomic sequence, for example to the corn Adh1 gene, as a positive control; c) performing a nucleic acid amplification reaction which results in no amplicon specific to elite event MZIR260 and results in an amplicon specific to the corn native gene positive control; and d) detecting an amplicon specific to the corn native gene positive control.

In another embodiment, the invention encompasses a method of detecting the presence of a nucleic acid molecule that is unique to elite event MZIR260 in a sample comprising corn nucleic acids, for example a biological sample, the method comprising: a) providing a genomic DNA sample from a corn plant that comprises a nucleic acid molecule that is unique to elite event MZIR260 (e.g., by isolating a nucleic acid molecule from corn); b) combining the nucleic acid molecule with a pair of polynucleotide primers of the invention and with a polynucleotide probe of the invention (e.g., comprising a nucleotide sequence of SEQ ID NO: 1, SEQ ID NO: 2, SEQ ID NO: 11 through 13, or a complement thereof); c) performing a nucleic acid amplification reaction which results in an amplicon which can be detected by the probe; and d) detecting the probe. In a further embodiment, the invention encompasses a DNA molecule comprising the amplicon produced by the methods of the invention. In a preferred aspect of this embodiment, the amplicon comprises a nucleotide sequence selected from the group consisting of SEQ ID NO: 1, SEQ ID NO: 2, SEQ ID NO: 3, SEQ ID NO: 4, and complements thereof.

In another embodiment, the invention encompasses a method of detecting the presence of DNA corresponding to the corn elite event MZIR260 in a biological sample, wherein the method comprises: (a) contacting the sample comprising DNA with a probe that hybridizes under high stringency conditions with genomic DNA from corn elite event MZIR260 and does not hybridize under high stringency conditions with DNA of a control corn plant; (b) subjecting the sample and probe to high stringency hybridization conditions; and (c) detecting hybridization of the probe to the DNA. In one aspect of this embodiment the probe comprises a nucleotide sequence selected from the group consisting of SEQ ID NO: 1, SEQ ID NO: 2, SEQ ID NO: 3, SEQ ID NO: 4, and complements thereof.

Detection can be by any means well known in the art including but not limited to fluorescent, chemiluminescent, radiological, immunological, or otherwise. In the case in which hybridization is intended to be used as a means for amplification of a particular sequence to produce an amplicon which is diagnostic for the corn elite event MZIR260 corn event, the production and detection by any means well known in the art of the amplicon is intended to be indicative of the intended hybridization to the target sequence where one probe or primer is utilized, or sequences where two or more probes or primers are utilized. The term “biological sample” is intended to comprise a sample that contains or is suspected of containing a nucleic acid molecule of elite event MZIR260, e.g., the junction sequences.

In yet another embodiment, the invention encompasses a kit for detecting the presence of corn elite event MZIR260 nucleic acids in a biological sample, wherein the kit comprises at least one nucleic acid molecule of sufficient length of contiguous nucleotides comprising or complementary to a nucleotide sequence selected from the group consisting of SEQ ID NO: 1, SEQ ID NO: 2, SEQ ID NO: 3, and SEQ ID NO: 4, that functions as a DNA primer or probe specific for corn elite event MZIR260, and other materials necessary to enable nucleic acid hybridization or amplification. A variety of detection methods can be used including TaqMan (ThermoFisher Scientific), genotyping-by-sequencing, thermal amplification, ligase chain reaction, southern hybridization, and colorimetric and fluorescent detection methods. In particular the invention provides for kits for detecting the presence of the target sequence, i.e., at least one of the junctions of elite event MZIR260, in a sample containing nucleic acid molecules from elite event MZIR260. The kit is comprised of at least one polynucleotide capable of binding to the target site (e.g., a junction sequence of elite event MZIR260) or substantially adjacent to the target site and at least one means for detecting the binding of the polynucleotide to the target site. The detecting means can be fluorescent, chemiluminescent, colorimetric, or isotopic and can be coupled at least with immunological methods for detecting the binding. A kit is also envisioned which can detect the presence of the target site in a sample, i.e., at least one of the junctions of corn elite event MZIR260, taking advantage of two or more polynucleotide sequences which together are capable of binding to nucleotide sequences adjacent to or within about 100 base pairs, or within about 200 base pairs, or within about 500 base pairs or within about 1000 base pairs of the target sequence and which can be extended toward each other to form an amplicon which contains at least the target site

In another embodiment, the invention encompasses a method for detecting eCry1Gb.1Ig protein in a biological sample, the method comprising: (a) extracting protein from a sample of corn elite event MZIR260 tissue; and (b) detecting the presence of eCry1Gb.1Ig protein. In some embodiments, the detecting comprises assaying the extracted protein using an immunological method comprising antibody specific for the eCry1Gb.1Ig protein produced by the corn elite event MZIR260 event; and (c) detecting the binding of said antibody to the eCry1Gb.1Ig protein.

In one embodiment, the invention provides a corn plant comprising elite event MZIR260 that controls at least one lepidopteran insect pest, including without limitation, one or more of the following Lepidopteran pests: Spodoptera spp. such as S. frugiperda (fall armyworm), S. littoralis (Egyptian cotton leafworm), S. ornithogalli (yellowstriped armyworm), S. praefica (western yellowstriped armyworm), S. eridania (southern armyworm), S. litura (Common cutworm/Oriental leafworm), S. cosmioides (black armyworm), S. exempta (African armyworm), S. mauritia (lawn armyworm) and/or S. exigua (beet armyworm), Ostrinia spp. such as O. nubilalis (European corn borer) and/or O. furnacalis (Asian corn borer), Plutella spp. such as P. xylostella (diamondback moth), Agrotis spp. such as A. ipsilon (black cutworm), A. segetum (common cutworm), A. gladiaria (claybacked cutworm), and/or A. orthogonia (pale western cutworm), Striacosta spp. such as S. albicosta (western bean cutworm), Helicoverpa spp. such as H. zea (corn earworm/soybean podworm), H. punctigera (native budworm), and/or H. armigera (cotton bollworm), Heliothis spp. such as H. virescens (tobacco budworm), Diatraea spp. such as D. grandiosella (southwestern corn borer) and/or D. saccharalis (sugarcane borer), Trichoplusia spp. such as T. ni (cabbage looper), Sesamia spp. such as S. nonagroides (Mediterranean corn borer), S. inferens (Pink stem borer) and/or S. calamistis (pink stem borer), Pectinophora spp. such as P. gossypiella (pink bollworm), Cochylis spp. such as C. hospes (banded sunflower moth), Manduca spp. such as M. sexta (tobacco hornworm) and/or M. quinquemaculata (tomato hornworm), Elasmopalpus spp. such as E. lignosellus (lesser cornstalk borer), Pseudoplusia spp. such as P. includens (soybean looper), Anticarsia spp. such as A. gemmatalis (velvetbean caterpillar), Plathypena spp. such as P. scabra (green cloverworm), Pieris spp. such as P. brassicae (cabbage butterfly), Papaipema spp. such as P. nebris (stalk borer), P seudaletia spp. such as P. unipuncta (common armyworm), Peridroma spp. such as P. saucia (variegated cutworm), Keiferia spp. such as K. lycopersicella (tomato pinworm), Artogeia spp. such as A. rapae (imported cabbageworm), Phthorimaea spp. such as P. operculella (potato tuberworm), Chrysodeixis spp. such as C. includens (soybean looper), Feltia spp. such as F. ducens (dingy cutworm), Chilo spp. such as C. suppressalis (striped stem borer), C. Agamemnon (oriental corn borer), C. venosatus (spotted borer), and C. partellus (spotted stalk borer), Cnaphalocrocis spp. such as C. medinalis (rice leaffolder), Conogethes spp. such as C. punctiferalis (Yellow peach moth), Mythimna spp. such as M. separata (Oriental armyworm), Athetis spp. such as A. lepigone (Two-spotted armyworm), Busseola spp. such as B. fusca (maize stalk borer), Etiella spp. such as E. zinckenella (pulse pod borer), Leguminivora spp. such as L. glycinivorella (soybean pod borer), Matsumuraeses spp. such as M. phaseoli (adzuki pod worm), Omiodes spp. such as O. indicata (Soybean leaffolder/Bean-leaf webworm), Rachiplusia spp. such as R. nu (sunflower Looper), Maruca spp. such as M. Testulalis Geyer (Bean pod borer), Monolepta spp. such as M. hieroglyphica (Double-spotted leaf beetle), or any combination of the foregoing.

In various embodiments, the corn elite event MZIR260 of the invention can be used for the control of Spodoptera frugiperda (fall armyworm), Diatraea saccharalis (sugarcane borer), Mythimna separata (oriental armyworm), Spodoptera litura (common cutworm/oriental leafworm), and Ostrinia furnacalis (Asian corn borer). In some embodiments, the elite event of the invention can be used for the control of a pest, e.g, fall armyworm, that is resistant to insect resistance trait protein(s), such as Vip3A protein (e.g., a Vip3Aa19 or Vip3Aa20, including without limitation maize event MIR162), a Cry1F protein (e.g., Cry1Fa, including without limitation maize event TC1507 or DP-4114), a Cry1A protein (e.g., Cry1A.105, including without limitation maize event MON89034), or a Cry2 protein (e.g., Cry2Ab, including without limitation maize event MON89034), a Cry1D protein (e.g., Cry1Da_7, Cry1Da2 or truncated Cry1Da, including without limitation maize event MON95379, DAS-01131-3 or ME240913), a Cry1B protein (e.g., Cry1B.868 or Cry1B.34, including without limitation maize event MON95379 or DP-910521-2).

In one embodiment, the invention provides a biological sample derived from an elite event MZIR260 corn plant, tissue, or seed, wherein the sample comprises a nucleotide sequence which is or is complementary to a sequence selected from the group consisting of SEQ ID NO: 1, SEQ ID NO: 2, SEQ ID NO: 3, SEQ ID NO: 4, SEQ ID NO: 5 and SEQ ID NO: 6, and wherein the sequence is detectable in the sample using a nucleic acid amplification or nucleic acid hybridization method. Thus, the genetic sequence may function a means of detection. In one aspect of this embodiment, the sample is selected from a corn commodity product, for example and not limited to corn flour, corn meal, corn syrup, corn oil, corn starch, and cereals manufactured in whole or in part to contain corn products. It is known in the art that a biological sample or extract may comprise proteins with biological activity. Therefore, in a further embodiment, the invention provides a biological sample derived from an elite event MZIR260 corn plant, tissue, or seed, wherein said biological sample comprises the insecticidal protein eCry1Gb.1Ig, which continues to have insecticidal activity.

In another embodiment, the invention provides an extract derived from an elite event MZIR260 corn plant, tissue, or seed comprising a nucleotide sequence which is or is complementary to a nucleotide sequence selected from the group consisting of SEQ ID NO: 1, SEQ ID NO: 2, SEQ ID NO: 3, SEQ ID NO: 4, SEQ ID NO: 5, and SEQ ID NO: 6. An example of such seed is deposited at the NCMA under Accession No. 202310010. In one aspect of this embodiment, the sequence is detected in the extract using a nucleic acid amplification or nucleic acid hybridization method. In some embodiments, the extract is a genomic DNA sample.

In another embodiment, the invention provides a method of producing a corn commodity product, comprising the steps of: a) obtaining transgenic elite event MZIR260 corn plant, cells or tissues thereof; and b) producing a corn commodity product from the said transgenic corn plant, cells, or tissue thereof, wherein the commodity product comprises protein concentrate, protein isolate, starch, meal, flour or oil therefrom.

In another embodiment, the invention provides a corn commodity product comprising a DNA molecule unique for corn elite event MZIR260, wherein said molecule comprises SEQ ID NO: 1, SEQ ID NO: 2, SEQ ID NO: 3, SEQ ID NO: 4, SEQ ID NO: 5 or SEQ ID NO: 6. In a further embodiment, the invention provides a non-living plant material comprising a nucleic acid molecule comprising SEQ ID NO: 1, SEQ ID NO: 2, SEQ ID NO: 3, SEQ ID NO: 4, SEQ ID NO: 5 or SEQ ID NO: 6. In some embodiments, the DNA molecule is detectable in the product or non-living plant material, e.g., using a nucleic acid amplification or nucleic acid hybridization method.

In another embodiment, the invention provides a method for determining zygosity of a corn plant comprising a corn elite event MZIR260 of the invention, said method comprising: (a) providing a genomic DNA sample from said corn plant; (b) producing a contacted sample by contacting said DNA sample with (i) a first event primer and a second event primer, wherein said first event primer specifically binds said transgene construct, said second event primer specifically binds said 5′ corn genomic flanking DNA or said 3′ corn genomic flanking DNA, and wherein said first event primer and said second event primer produce an event amplicon which is unique to elite event MZIR260, when subjected to quantitative PCR conditions, (ii) at least one native insertion site first primer and at least one native insertion site second primer, wherein the first primer is a forward primer and the second primer is a reverse primer, wherein a first and second primer function together when subjected to quantitative PCR conditions to produce an amplicon from the native MZIR260 insertion site when elite event MZIR260 is not present in the genome, (iii) an event probe that hybridizes with said event amplicon, (iv) a native insertion site probe that hybridizes with said native insertion site amplicon; (c) quantitating said event probe that hybridized to said event amplicon and quantitating said native insertion site probe that hybridized to said native insertion site amplicon (e.g., using fluorescence-based endpoint quantitative PCR); (d) comparing amounts of hybridized event probe to hybridized native insertion site probe; and (e) determining zygosity of said corn plant comprising corn elite event MZIR260 by comparing ratios of hybridized event probe and hybridized native insertion site probe. In some embodiments, the probes are fluorescent. The event primer set and probe and native insertion site primer set and probe may be mixed with the same DNA sample, or they may be separate with different DNA samples derived from the same corn plant. The native insertion primer set may comprise more than one forward native insertion site primer and/or more than one reverse native insertion site primer. The quantification of the event probe and the native insertion site probe (e.g., using fluorescence) may be sequentially or simultaneously. The results indicate if the corn plant is homozygous for elite event MZIR260 (i.e., has positive results for the event endpoint quantitative PCR but not for the native insertion site endpoint quantitative PCR), is heterozygous for elite event MZIR260 (i.e., has a positive result for both the event and for the native insertion site endpoint quantitative PCRs) or is wild type (i.e., has positive results for the native insertion site endpoint quantitative PCR but not for the event endpoint quantitative PCR).

It will be recognized that some sequence diversity will be found for the native insertion site, based on genetic diversity of the various corn germplasms into which elite event MZIR260 is introduced. Therefore, for successful zygosity determination in novel corn germplasms, a native insertion site primer set may need to be identified so that an amplicon is produced when elite event MZIR260 is not present in the genome, when subjected to quantitative PCR conditions. Multiple native insertion site primer sets and probes may be needed to properly determine the zygosity of event MZIR260 in a variety of corn germplasms. Multiple native insertion site primer sets and/or probes may be included in a single reaction to produce a native insertion site amplicon that hybridizes with a native insertion site probe. Similarly, event primers which specifically bind to the 5′ or 3′ flanking sequence of the MZIR260 may need to be identified for successful zygosity determination in novel corn germplasms, as the 5′ and/or 3′ flanking sequences may be diverse among a variety of germplasms. A gain, multiple event primer sets may be included in a single reaction to produce an event amplicon unique to elite event MZIR260.

In a further embodiment of the method for determining zygosity described above, the event and/or native insertion site amplicon may consist of 50-200 nucleotides in length. In a preferred embodiment, the amplicon for the event and for the native insertion site is 50-150 nucleotides in length. In another embodiment, the first event primer comprises at least 10 contiguous nucleotides of the sequence set forth in SEQ ID NO: 7, or a complement thereof, and the second event primer comprises at least 10 contiguous nucleotides of the sequence as set forth in SEQ ID NO: 8 or SEQ ID NO: 9, or a complement thereof. In a further embodiment, the first event primer is selected from SEQ ID NOs: 11, 17, 20, 28, and 32, or a complement thereof. In another embodiment, the second event primer is selected from SEQ ID NO: 12, 18, 21, 29, or 33, or a complement thereof. In another embodiment, the first native insertion site primer comprises at least 10 contiguous nucleotides of SEQ ID NO: 8, or a complement thereof, and the second native insertion site primer comprises at least 10 contiguous nucleotides of SEQ ID NO: 9, or a complement thereof. It is recognized that there may be more than one first primer or second primer for the native insertion site primers.

In a further embodiment, the results of the method for determining zygosity described above are read directly in a plate reader. The present invention also encompasses a kit for performing the method of determining zygosity described above. The kit comprises all primers and probes needed for performing the zygosity assay on a DNA sample, including a first event primer, a second event primer, at least one native insertion site primer, at least one native insertion site reverse primer, an event probe, and a native insertion site probe. The kit may include more than one primer set/probe for the event, for the native insertion site, or both. The kit may also include more than one pair of primers, for example two forward primers and a single reverse primer, as described in Example 2.

In a further embodiment, the present invention encompasses a method of breeding a corn plant comprising elite event MZIR260 wherein the zygosity of a corn plant comprising corn elite event MZIR260 is determined by the method described above. The zygosity determination method may be used in a breeding program to determine the zygosity of the event MZIR260 in a segregating progeny population. Corn plants may then be selected which are homozygous for event MZIR260 based on the results of the zygosity determination method described above. Corn plants may also be discarded if they are found to be heterozygous for event MZIR260 based on the results of the zygosity determination method.

In yet another embodiment, the invention provides a method for producing a corn plant useful for control of insects comprising: (a) sexually crossing a first parent corn plant with a second parent corn plant, wherein said first or second parent corn plant comprises elite event MZIR260, thereby producing a plurality of first generation progeny plants; and (b) selecting from the progeny plants a plant that has insect resistance to fall armyworm and/or sugarcane borer; wherein the progeny plants comprise a nucleotide sequence selected from the group consisting of SEQ ID NO: 1, SEQ ID NO: 2, SEQ ID NO:3, SEQ ID NO: 4, SEQ ID NO: 5, and SEQ ID NO: 6.

In yet another embodiment, the invention provides a use of elite event MZIR260 to confer insect resistance to a corn plant lacking said event. This usage may comprise, for example, sexually crossing a parent corn plant comprising elite event MZIR260 with a second parent corn plant which does not comprise elite event MZIR260 and selecting for progeny which comprise elite event MZIR260. The selection may be done, e.g., using a PCR or probe-based method as described herein or using a phenotypic assay, e.g., selecting for progeny that have insect resistance to fall armyworm and/or sugarcane borer. The progeny may further be backcrossed to the second parent, optionally multiple times, or crossed with additional corn plants as part of a breeding program to produce at least one variety of corn comprising elite event MZIR260 which previously did not comprise said event.

In another embodiment, the invention provides a method of asexually propagating corn plant comprising elite event MZIR260. A sexual propagation of a corn plant may be performed using methods well-known in the art, for example by anther culture or by microspore-derived plant tissue culture. In vitro plant regeneration may be performed by micropropagation, which involves the suppression of apical dominance resulting in the activation and multiplication of axillary buds, or by somatic embryogenesis, where, for example, cotyledon containing embryos are formed from somatic cells. A sexual propagation and in vitro plant regeneration are needed for asexual reproduction. In a further embodiment, the invention provides a corn plant comprising elite event MZIR260 produced by asexual propagation. The invention also provides use of a corn plant comprising elite event MZIR260 plant, cells, or tissues to produce a corn plant comprising elite event MZIR260.

In another embodiment, the invention provides a method of producing hybrid corn seeds comprising: a) crossing a first corn plant comprising elite event MZIR260 (e.g., a first inbred parent line) with a second corn plant that is different from the first corn plant (e.g., a second inbred parent line not comprising elite event MZIR260) to produce hybrid corn seed; and b) harvesting the hybrid corn seeds produced. In some embodiments, the method comprises crossing a first inbred corn line comprising elite event MZIR260 with a second inbred corn line having a different genotype (e.g., not comprising elite event MZIR260), wherein at least one of the corn lines is male sterile (e.g., via mechanical emasculation or via cytoplasmic male sterility). In some embodiments, the method comprises (a) planting seeds of a first inbred corn line comprising elite event MZIR260, and seeds of a second inbred line having a different genotype than the first inbred corn plant; (b) cultivating corn plants resulting from said planting until time of flowering and the production of flowers; (c) emasculating said flowers of plants of either the first or the second corn inbred line; (d) sexually crossing the two different inbred lines with each other by pollinating the emasculated plant with pollen of the non-emasculated plant; and (e) allowing hybrid seed to be produced by the emasculated plant and harvesting the hybrid seed produced thereby. In one aspect of this embodiment, the first inbred corn line is the female parent. In another aspect of this embodiment, the first inbred corn line is the male parent. The invention also encompasses the hybrid seed produced by the embodied methods and hybrid plants grown from the seed.

In another embodiment, the invention provides a method of selecting markers associated with corn elite event MZIR260 comprising: (a) screening corn elite event MZIR260 chromosome 2 sequences from a first corn genome, (b) comparing these with a non-transgenic isogenic line genome sequences, (c) comparing the sequences for the purpose of detecting sequence variations, (d) using these sequence variations as a means to develop markers associated with corn elite event MZIR260, (e) using the markers to screen lines, and (f) detecting marker(s) confirming the presence of corn elite event MZIR260 sequences on chromosome 2. In another embodiment, the invention provides a method of marker assisted selection for elite event MZIR260 comprising: (a) isolating nucleic acid molecule(s), or preparing a nucleic acid sample, from corn; (b) combining the nucleic acid molecule(s) with a pair of polynucleotide primers and probes, selected from the group comprising SEQ ID NO: 1, SEQ ID NO: 2, SEQ ID NO: 11, SEQ ID NO: 12, SEQ ID NO: 13, or any of SEQ ID NOs: 11, 12, 13, 17, 18, 19, 20, 21, 28, 29, 32, or 33, or their complements; (c) performing a nucleic acid amplification reaction which results in an amplicon; (d) detecting the amplicon; and (e) selecting the plant for the purposes of breeding insect resistant corn comprising corn elite event MZIR260.

In another embodiment, the invention comprises a transgenic corn plant, cells, or tissues comprising elite event MZIR260, characterized by the presence of SEQ ID NO: 1, SEQ ID NO: 2, SEQ ID NO: 3, SEQ ID NO: 4, SEQ ID NO: 5, or SEQ ID NO: 6, wherein the transgenic corn plant, cells, or tissues are further defined as a progeny or derived from a progeny of any generation of a corn plant comprising elite event MZIR260. In a further embodiment, the transgenic corn plant, cells, or tissues are or are derived from a hybrid bred from at least one parent comprising elite event MZIR260.

In some embodiments, an elite event MZIR260 of the disclosure can be used to develop other corn plants with one or more additional desired traits in addition to the insect resistance provided by the eCry1Gb.1Ig protein by, for example, breeding elite event MZIR260 with other events known in the art to have the desired trait, or by targeted insertion of an expression cassette comprising one or more gene(s) conferring the trait(s). Such stacked combinations in plants can also be created by other methods including, but not limited to, cross breeding plants by any conventional methodology or further transforming a plant or plant part comprising the MZIR260 event with an expression cassette comprising one or more gene(s) conferring the trait(s). It is further recognized that polynucleotides can be stacked at a desired genomic location using a site-specific nuclease or recombination system (e.g., Flp/FRT, Cre/Lox, TALE-endonucleases, zinc finger nucleases, CRISPR/Cas and related technologies). See U.S. Pat. Nos. 7,214,536, 8,921,332, 8,765,448, 5,527,695, 5,744,336, 5,910,415, 6,110,736, 6,175,058, 6,720,475, 6,455,315, 6,458,594 and US Patent Publication Nos. US2019093090, US2019264218, US2018327785, US2017240911, US2016208272, US2019062765.

In some embodiments, the one or more desired traits can include one or more traits conferred by polypeptides or double stranded RNA molecules (dsRNA) of interest for agronomic traits that primarily are of benefit to a seed company, grower or grain processor. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

Polynucleotides conferring resistance/tolerance to an herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (AC Case).

Polypeptides encoded by nucleotides sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional suitable polynucleotides include those coding for insecticidal polypeptides. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62: 807-813).

In embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

Double stranded RNA molecules useful with the disclosure include but are not limited to those that suppress target insect genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

In some embodiments, the one or more additional desired traits is conferred by any one or more of the insecticidal proteins or dsRNAs present in any of the following events: the Bt11 event (see U.S. Pat. No. 6,114,608), the MIR604 event (see U.S. Pat. No. 8,884,102), the MIR162 event (see U.S. Pat. No. 8,232,456), the 5307 event (see U.S. Pat. No. 10,428,393), the MZIR098 event (see US Patent Application No. US20200190533), the TC1507 event (see U.S. Pat. No. 7,288,643), the DAS-59122-7 event (see U.S. Pat. No. 7,323,556), the MON810 event (see U.S. Pat. No. 6,713,259), the MON863 event (see U.S. Pat. No. 7,705,216), the MON89034 event (see U.S. Pat. No. 8,062,840), the MON88017 event (see U.S. Pat. No. 9,556,492), the DP-4114 event (see U.S. Pat. No. 9,725,772), the MON87411 event (see U.S. Pat. No. 9,441,240), the DP-032218-9 event (see US Patent Application No. US2015361447), the DP-033121-3 event (see US Patent Application No. US2015361446), the DP-023211-2 event (see PCT Publication No. WO2019209700), the MON95379 event (see US Patent Application No. US2020032289), the DBN9936 event (see PCT Publication No. WO 2016173361), the DBN9501 event (see PCT Publication No. WO20207125), the GH 5112E-117C event (see PCT Publication No. WO17/088480), LP007-1 (see Chinese Patent Application No. CN112852801), LP007-2 (Chinese Patent Application No. CN112831584), LP007-3 (Chinese Patent Application No. CN112877454), LP007-4 (Chinese Patent Application No. CN112831585), LP007-5 (Chinese Patent Application No. CN113151534), LP007-6 (Chinese Patent Application No. CN113151533), LP007-7 (Chinese Patent Application No. CN112852991), LP007-8 (CN113980958), Ruifeng8, ND207, or the Ruifeng125 event (see Chinese Patent Application No. CN105017391), the KJ 1172 event (see Chinese Patent Application No. CN116410977 A), the LD 02 event (see Chinese Patent Application No. CN115820630A), the LD05 event (See Chinese Patent Application No. CN116287384), the LG11 event (see Chinese Patent Application Nos. CN116200529A and CN115725571A), the DBN9235 event (see Chinese Patent Application No. CN116219063), the DBN9508 event (see Chinese Patent No. CN109971880), the DBN9888 event (see PCT Publication No. WO16/173540), the DBN9978 event (see PCT Publication No. WO16/173362), the DBN9953 event (see Chinese Patent No. CN104878092), the DBN9927 event (see PCT Publication No. WO16/173360), the LP026-1 event (see Chinese Application No. CN116144672A), the LP026-2 event (see Chinese Application No. CN116144818A), the LP026-3 event (see Chinese Application No. CN116144671A), the LP026-4 event (see Chinese Application No. CN116144817A), the LP026-5 event (see Chinese Application No. CN116200519A), the 2A-7 event (see Chinese Patent Application No. CN112280743A), the CA 09328 event (see Chinese Patent Application No. CN112126707A), the ZM8-143 event (see Chinese Patent Application No. CN108018286), the ZM1-027 event (see Chinese Patent Application No. CN108018368A), the DP-915635-4 event (see PCT Publication No. WO21/247204), the DP-910521-2 event (see PCT Publication No. WO23/091888), the DAS-01131-3 event (see PCT Publication No. WO23/091884), the MON95275 event (see PCT Publication No. WO21/216571), and the M E 240913 event (see PCT Publication No. WO21/087586).

In some embodiments, a corn plant (including a tissue, seed or cell thereof) comprising elite event MZIR260 further comprises one or more of the following insect resistance events: the Bt11 event (see U.S. Pat. No. 6,114,608), the MIR604 event (see U.S. Pat. No. 8,884,102), the MIR162 event (see U.S. Pat. No. 8,232,456), the 5307 event (see U.S. Pat. No. 10,428,393), the MZIR098 event (see US Patent Application No. US20200190533), the TC1507 event (see U.S. Pat. No. 7,288,643), the DAS-59122-7 event (see U.S. Pat. No. 7,323,556), the MON810 event (see U.S. Pat. No. 6,713,259), the MON863 event (see U.S. Pat. No. 7,705,216), the MON89034 event (see U.S. Pat. No. 8,062,840), the MON88017 event (see U.S. Pat. No. 9,556,492), the DP-4114 event (see U.S. Pat. No. 9,725,772), the MON87411 event (see U.S. Pat. No. 9,441,240), the DP-032218-9 event (see US Patent Application No. US2015361447), the DP-033121-3 event (see US Patent Application No. US2015361446), the DP-023211-2 event (see PCT Publication No. WO2019209700), the MON95379 event (see US Patent Application No. US2020032289), the DBN9936 event (see PCT Publication No. WO 2016173361), the DBN9501 event (see PCT Publication No. WO20207125), the GH 5112E-117C event (see PCT Publication No. WO17/088480), LP007-1 (see Chinese Patent Application No. CN112852801), LP007-2 (Chinese Patent Application No. CN112831584), LP007-3 (Chinese Patent Application No. CN112877454), LP007-4 (Chinese Patent Application No. CN112831585), LP007-5 (Chinese Patent Application No. CN113151534), LP007-6 (Chinese Patent Application No. CN113151533), LP007-7 (Chinese Patent Application No. CN112852991), LP007-8 (CN113980958), Ruifeng8, ND207, or the Ruifeng125 event (see Chinese Patent Application No. CN105017391), the KJ 1172 event (see Chinese Patent Application No. CN116410977 A), the LD 02 event (see Chinese Patent Application No. CN115820630A), the LD05 event (See Chinese Patent Application No. CN116287384), the LG11 event (see Chinese Patent Application Nos. CN116200529A and CN115725571A), the DBN9235 event (see Chinese Patent Application No. CN116219063), the DBN9508 event (see Chinese Patent No. CN109971880), the DBN9888 event (see PCT Publication No. WO16/173540), the DBN9978 event (see PCT Publication No. WO16/173362), the DBN9953 event (see Chinese Patent No. CN104878092), the DBN9927 event (see PCT Publication No. WO16/173360), the LP026-1 event (see Chinese Application No. CN116144672A), the LP026-2 event (see Chinese Application No. CN116144818A), the LP026-3 event (see Chinese Application No. CN116144671A), the LP026-4 event (see Chinese Application No. CN116144817A), the LP026-5 event (see Chinese Application No. CN116200519A), the 2A-7 event (see Chinese Patent Application No. CN112280743A), the CA 09328 event (see Chinese Patent Application No. CN112126707A), the ZM8-143 event (see Chinese Patent Application No. CN108018286), the ZM1-027 event (see Chinese Patent Application No. CN108018368A), the DP-915635-4 event (see PCT Publication No. WO21/247204), the DP-910521-2 event (see PCT Publication No. WO23/091888), the DAS-01131-3 event (see PCT Publication No. WO23/091884), the MON95275 event (see PCT Publication No. WO21/216571), and the ME240913 event (see PCT Publication No. WO21/087586).

In some embodiments, one or more other nucleic acids of interest encode one or more herbicide tolerance agents, e.g., PAT (phosphinothricin N-acetyltransferase), AAD-1 (aryloxyalkanoate dioxygenase 1), EPSPS (5-enolpyruvulshikimate-3-phosphate synthase), DMO (dicamba mono-oxygenase), 2,4-D and FOPs dioxygenase, or inhibitors of protoporphyrinogen oxidase (PPO, see, e.g., US Patent Application No. US2019185873).

In some embodiments, a corn plant (including a tissue, seed or cell thereof) comprising elite event MZIR260 further comprises any one or more of the following herbicide tolerance events: GA 21 (see PCT Publication No. WO98/44140), NK 603 (see U.S. Pat. No. 6,825,400), DAS40278 (see PCT Publication No. WO2011/022469), DBN9858 (see PCT Publication No. WO2016173508), MON87429 (see PCT Publication No. WO19/152316), Zm_CSM 63715L (see PCT Publication No. WO2024137408); W2-2 (see Chinese Patent Application No. CN113278721), T25 (see USDA/APHIS Petition 94-357-01 for Determination of Nonregulated Status for Glufosinate Resistant Corn Transformation Events T14 and T25, June 1995), MON87429 (see PCT Publication No. WO19/152316), and Zm_CSM 63715L in combination with any other event, including MON87429.

In some embodiments, one or more other nucleic acids of interest encode one or more enzymes, e.g., an alpha-amylase. In some embodiments, a corn plant comprising elite event MZIR260 further comprises the 3272 event (see U.S. Pat. No. 7,635,799).

In some embodiments, a corn plant (including a tissue, seed or cell thereof) comprising elite event MZIR260 further comprises two or more insect resistance and/or herbicide tolerance events, such as the events described above.

In some embodiments, one or more other nucleic acids of interest comprise one or more of the following events: MZDT09Y (see U.S. Pat. No. 9,121,033), LY038 (see U.S. Pat. No. 7,157,281), BT176 (see Koziel et al. (1993) Biotechnology 11:194-200), and DP202216-6 (see US Patent Application No US2019320607).

In another embodiment, the invention encompasses a process for producing corn elite event MZIR260 seed. This process comprises crossing a corn plant comprising elite event MZIR260 of the invention with a second corn plant. The second corn plant may or may not comprise the MZIR260 event. In some embodiments, the second plant comprises at least one other event that confers a desired trait, e.g., one or more of the events listed above. In preferred embodiments, the second corn plant does not comprise the MZIR260 event. In some embodiments, following the crossing, or pollination event, the seed is allowed to develop and set in the maternal plant. The invention further comprises seed of corn comprising elite event MZIR260 produced by the process described above, as well as the corn plant comprising elite event MZIR260 produced by germinating the seed.

In another embodiment, the invention provides a process of introducing an additional trait into a corn plant comprising elite event MZIR260, comprising: (a) crossing a corn plant comprising elite event MZIR260, with another maize plant that comprises an additional trait to produce hybrid progeny plants, (b) selecting hybrid progeny plants that have the additional trait and elite event MZIR260 to produce selected hybrid progeny plants; (c) backcrossing the selected progeny plants with either the corn elite event MZIR260 parental plants or the parent plants containing the additional trait to produce backcross progeny plants; (d) selecting for backcross progeny plants that have the additional trait and elite event MZIR260 to produce selected backcross progeny plants; and (e) repeating steps (c) and (d) at least three or more times to produce backcross progeny plants that comprise the additional trait and elite event MZIR260. The invention further comprises a plant produced by the process described above.

In another embodiment, the invention provides a method for developing a corn elite event MZIR260 plant germplasm in a corn plant breeding program, comprising applying plant breeding techniques wherein said techniques comprise recurrent selection, backcrossing, pedigree breeding, marker enhanced selection, haploid/double haploid production, or transformation of a corn plant comprising elite event MZIR260, or its parts, wherein application of said techniques results in development of a second corn germplasm comprising elite event MZIR260.

In another embodiment, the invention provides a method of producing a corn elite event MZIR260 plant with doubled haploid chromosomes, the method comprising: (a) crossing a plant comprising elite event MZIR260 with an inducer maize plant to produce a progeny with haploid chromosomes; and (b) doubling the haploid chromosomes in the progeny to produce a corn elite event MZIR260 plant with doubled haploid chromosomes.

In various embodiments, the elite event MZIR260 can be introgressed in any corn inbred or hybrid using art recognized breeding techniques. The goal of plant breeding is to combine in a single variety or hybrid various desirable traits. For field crops, these traits may include resistance to insects and diseases, tolerance to herbicides, tolerance to heat and drought, reducing the time to crop maturity, greater yield, and better agronomic quality. With mechanical harvesting of many crops, uniformity of plant characteristics such as germination and stand establishment, growth rate, maturity, and plant and ear height, is important.

Field crops are bred through techniques that take advantage of the plant's method of pollination. A plant is self-pollinated if pollen from one flower is transferred to the same or another flower of the same plant. A plant is cross-pollinated if the pollen comes from a flower on a different plant.

Plants that have been self-pollinated and selected for type for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of hybrid plants that differ genetically and will not be uniform.

Corn can be bred by both self-pollination and cross-pollination techniques. Corn has separate male and female flowers on the same plant, located on the tassel and the ear, respectively. Natural pollination occurs in corn when wind blows pollen from the tassels to the silks that protrude from the tops of the ears.

A reliable method of controlling male fertility in plants offers the opportunity for improved plant breeding. This is especially true for development of corn hybrids, which relies upon some sort of male sterility system. There are several options for controlling male fertility available to breeders, such as: manual or mechanical emasculation (or detasseling), cytoplasmic male sterility, genetic male sterility, gametocides and the like.

Hybrid corn seed is typically produced by a male sterility system incorporating manual or mechanical detasseling or cytoplasmic male sterility. For example, alternate strips of two corn inbreds are planted in a field, and the pollen-bearing tassels are removed from one of the inbreds (female) or the inbred is cytoplasmic male sterile. Providing that there is sufficient isolation from sources of foreign corn pollen, the ears of the detasseled inbred will be fertilized only from the other inbred (male), and the resulting seed is therefore hybrid and will form hybrid plants.

### Development of Corn Inbred Lines

The use of male sterile inbreds is but one factor in the production of corn hybrids. Plant breeding techniques known in the art and used in a corn plant breeding program include, but are not limited to, recurrent selection, backcrossing, pedigree breeding, restriction length polymorphism enhanced selection, marker assisted selection and transformation. The development of corn hybrids in a corn plant breeding program requires, in general, the development of homozygous inbred lines, the crossing of these lines, and the evaluation of the crosses. Pedigree breeding and recurrent selection breeding methods are used to develop inbred lines from breeding populations. Corn plant breeding programs combine the genetic backgrounds from two or more inbred lines or various other germplasm sources into breeding pools from which new inbred lines are developed by selfing and selection of desired phenotypes. The new inbreds are crossed with other inbred lines and the hybrids from these crosses are evaluated to determine which of those have commercial potential. Plant breeding and hybrid development, as practiced in a corn plant-breeding program, are expensive and time-consuming processes.

Pedigree breeding starts with the crossing of two genotypes, each of which may have one or more desirable characteristics that is lacking in the other or which complements the other. If the two original parents do not provide all the desired characteristics, other sources can be included in the breeding population. In the pedigree method, superior plants are selfed and selected in successive generations. In the succeeding generations the heterozygous condition gives way to homogeneous lines as a result of self-pollination and selection. Typically in the pedigree method of breeding five or more generations of selfing and selection is practiced: F₁→F₂; F₂→F₃; F₃→F₄; F₄→F₋₅; etc.

Recurrent selection breeding, backcrossing for example, can be used to improve an inbred line and a hybrid that is made using those inbreds. Backcrossing can be used to transfer a specific desirable trait from one inbred or source to an inbred that lacks that trait. This can be accomplished, for example, by first crossing a superior inbred (recurrent parent) to a donor inbred (non-recurrent parent), that carries the appropriate gene(s) for the trait in question. The progeny of this cross is then mated back to the superior recurrent parent followed by selection in the resultant progeny for the desired trait to be transferred from the non-recurrent parent. After five or more backcross generations with selection for the desired trait, the progeny will be homozygous for loci controlling the characteristic being transferred, but will be like the superior parent for essentially all other genes. The last backcross generation is then selfed to give pure breeding progeny for the gene(s) being transferred. A hybrid developed from inbreds containing the transferred gene(s) is essentially the same as a hybrid developed from the same inbreds without the transferred gene(s).

An inbred plant could also be produced by applying double haploid methods to the progeny of a cross between a corn plant comprising elite event MZIR260 and a different plant. Double haploid methods produce substantially homozygous plants without repeated backcrossing steps. The haploid/doubled haploid process of developing inbreds starts with the induction of a haploid by using, for example, KWS inducers lines, Krasnador inducers lines, stock six inducer lines (Coe, 1959, Am. Nat. 93:381-382). The haploid cell is then doubled, and the doubled haploid plant is produced. In some embodiments, the invention is a method of producing a corn plant with doubled haploid chromosomes derived from a corn elite event MZIR260, the method comprising: (a) crossing a plant, wherein said plant comprises elite event MZIR260, with an inducer maize plant to produce a progeny with haploid chromosomes; and (b) doubling the haploid chromosomes in the progeny to produce a maize plant with doubled haploid chromosomes. In some embodiments, the progeny may be for example a cell, seed, embryo or plant. In further embodiments, the maize plant with doubled haploid chromosomes produced by step (b) above is a maize inbred plant with the characteristics of corn elite event MZIR260. In other embodiments, the plant crossed with an inducer in step (a) is a hybrid maize plant produced by crossing a corn plant comprising elite event MZIR260 with a different plant.

For examples of the use of double hybrid methods, see Prasanna et al. (eds) Doubled Haploid Technology in Maize Breeding: Theory and Practice Mexico, D.F.: CIMMYT, Barnabus et al. “Colchicine, an efficient genome doubling agent for maize microsporescultured in anthero”, Plant Cell Reports, 1999, 18:858-862 or US patent publication 2003/0005479.

Sometimes this doubled haploid can be used as an inbred but sometimes it is further self pollinated to finish the inbred development. Another breeding process is pedigree selection which uses the selection in an F2 population produced from a cross of two genotypes (often elite inbred lines), or selection of progeny of synthetic varieties, open pollinated, composite, or backcrossed populations. Pedigree selection is effective for highly heritable traits, such as a transgenic event, but other traits, such as yield, require replicated test crosses at a variety of stages for accurate selection.

Elite inbred lines, that is, pure breeding, homozygous inbred lines, can also be used as starting materials for breeding or source populations from which to develop other inbred lines. These inbred lines derived from elite inbred lines can be developed using the pedigree breeding and recurrent selection breeding methods described earlier. As an example, when backcross breeding is used to create these derived lines in a corn plant-breeding program, elite inbreds can be used as a parental line or starting material or source population and can serve as either the donor or recurrent parent.

### Development of Corn Hybrids

A single cross corn hybrid results from the cross of two inbred lines, each of which has a genotype that complements the genotype of the other. The hybrid progeny of the first generation is designated F₁. In the development of commercial hybrids in a corn plant-breeding program, only the F₁ hybrid plants are sought. Preferred F₁ hybrids are more vigorous than their inbred parents. This hybrid vigor, or heterosis, can be manifested in many polygenic traits, including increased vegetative growth and increased yield.

In various embodiments, the development of a corn hybrid breeding program involves three steps: (1) the selection of plants from various germplasm pools for initial breeding crosses; (2) the selfing of the selected plants from the breeding crosses for several generations to produce a series of inbred lines, which, although different from each other, breed true and are highly uniform; and (3) crossing the selected inbred lines with different inbred lines to produce the hybrid progeny (F₁). During the inbreeding process in corn, the vigor of the lines decreases. Vigor is restored when two different inbred lines are crossed to produce the hybrid progeny (F₁). An important consequence of the homozygosity and homogeneity of the inbred lines is that the hybrid between a defined pair of inbreds will always be the same. Once the inbreds that give a superior hybrid have been identified, the hybrid seed can be reproduced indefinitely as long as the homogeneity of the inbred parents is maintained. Much of the hybrid vigor exhibited by F₁ hybrids is lost in the next generation (F₂). Consequently, seed from hybrids is not used for planting stock.

Hybrid seed production requires elimination or inactivation of pollen produced by the female parent. Incomplete removal or inactivation of the pollen provides the potential for self-pollination. This inadvertently self-pollinated seed may be unintentionally harvested and packaged with hybrid seed.

Once the seed is planted, it is possible to identify and select these self-pollinated plants. These self-pollinated plants will be genetically equivalent to the female inbred line used to produce the hybrid.

As is readily apparent to one skilled in the art, the foregoing are only some of the various ways by which the inbred of the invention can be obtained by those looking to introgress the transgenic genotype of the invention into other corn lines. Other means are available, and the above examples are illustrative only.

One skilled in the art will also recognize that transgenic corn seed comprising elite event MZIR260 be treated with various seed-treatment or plant-treatment chemicals, including insecticides and/or fungicides. In one embodiment, the invention comprises a method for protecting a corn plant comprising elite event MZIR260 against feeding damage by one or more pests, said method comprising (a) providing a corn seed or plant comprising elite event MZIR260; and (b) treating the seed or plant with an insecticide. The present invention also encompasses a corn plant comprising elite event MZIR260 treated with an insecticide. The present invention also encompasses a corn seed comprising elite event MZIR260 treated with an insecticide. In some embodiments, the insecticide is or comprises one or more of the following: (7E,9Z)-dodeca-7,9-dien-1-yl acetate, (9Z,11E)-tetradeca-9,11-dien-1-yl acetate, (9Z,12E)-tetradeca-9,12-dien-1-yl acetate, (E)-6-methylhept-2-en-4-ol, (E)-dec-5-en-1-yl acetate with (E)-dec-5-en-1-ol, (E)-tridec-4-en-1-yl acetate, (E,Z)-tetradeca-4,10-dien-1-yl acetate, (Z)-dodec-7-en-1-yl acetate, (Z)-hexadec-11-en-1-yl acetate, (Z)-hexadec-11-enal, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, 1,2-dibromo-3-chloropropane, 1,2-dichloropropane, 1,2-dichloropropane with 1,3-dichloropropene, 1,3-dichloropropene, 14-methyloctadec-1-ene, 1-hydroxy-1H-pyridine-2-thione, 2-(octylthio)-ethanol, 2-chlorophenyl N-methylcarbamate (CPM C), 3-(4-chlorophenyl)-5-methylrhodanine, 3,4-dichlorotetrahydrothio-phene 1,1-dioxide, 4-(quinoxalin-2-ylamino)benzenesulfonamide, 4-methylnonan-5-ol with 4-methylnonan-5-one, 5-methyl-6-thioxo-1,3,5-thiadiazinan-3-ylacetic acid, 6-isopentenylaminopurine, 8-hydroxyquinoline sulfate, abamectin, acequinocyl, acetamiprid, acetoprole, acrinathrin, acynonapyr, Adoxophyes orana GV, afidopyropen, afoxolaner, Agrobacterium radiobacter, AKD-3088, alanycarb, aldicarb, aldoxycarb, allethrin, alpha-cypermethrin, alphamethrin, alpha-multistriatin, Amblyseius spp., amidoflumet, amino acids, aminocarb, Anagrapha falcifera NPV, Anagrus atomus, Aphelinus abdominalis, Aphidius colemani, Aphidoletes aphidimyza, apholate, Autographa californica NPV, AZ 60541, azadirachtin, azocyclotin, Bacillus aizawai, Bacillus chitinosporus AQ746 (NRRL Accession No B-21 618), Bacillus firmus, Bacillus kurstaki, Bacillus mycoides AQ726 (NRRL Accession No. B-21664), Bacillus pumilus (NRRL Accession No B-30087), Bacillus pumilus AQ717 (NRRL Accession No. B-21662), Bacillus sp. AQ175 (ATCC Accession No. 55608), Bacillus sp. AQ177 (ATCC Accession No. 55609), Bacillus sp. AQ178 (ATCC Accession No. 53522), Bacillus sphaericus Neide, Bacillus subtilis AQ153 (ATCC Accession No. 55614), Bacillus subtilis AQ30002 (NRRL Accession No. B-50421), Bacillus subtilis AQ30004 (NRRL Accession No. B-50455), Bacillus subtilis AQ713 (NRRL Accession No. B-21661), Bacillus subtilis AQ743 (NRRL Accession No. B-21665), Bacillus subtilis unspecified, Bacillus thuringiensis AQ52 (NRRL Accession No. B-21619), Bacillus thuringiensis BD #32 (NRRL Accession No B-21530), Bacillus thuringiensis Berliner, Bacillus thuringiensis subsp. Aizawai, Bacillus thuringiensis subsp. Israelensis, Bacillus thuringiensis subsp. Japonensis, Bacillus thuringiensis subsp. Kurstaki, Bacillus thurin-giensis subsp. Tenebrionis, Bacillus thuringiensis subspec. kurstaki BMP 123, Beauveria bassiana, Beauveria brongniartii, benclothiaz, benomyl, bensultap, benzoximate, benzpyrimoxan, betacyfluthrin, beta-cypermethrin, bethoxazin, bifenazate, bifenthrin, binapacryl, bioallethrin, bioresmethrin, bis(tributyltin) oxide, bisazir, bistrifluron, bisulflufen, brevicomin, broflanilide, brofluthrinate, bromoacetamide, bromophos-ethyl, bronopol, busulfan, butocarboxim, butopyronoxyl, butoxy (polypropylene glycol), butylpyridaben, cadusafos, calcium arsenate, carbaryl, carbofuran, carbon disulfide, carbosulfan, cartap, CAS number: 1594624-87-9, CAS number: 1922957-47-8, CAS number: 1255091-74-7, CAS number: 1365070-72-9, CAS number: 1445683-71-5, CAS number: 1445684-82-1, CAS number: 1594626-19-3, CAS number: 1594637-65-6, CAS number: 1632218-00-8, CAS number: 1808115-49-2, CAS number: 1922957-46-7, CAS number: 1922957-48-9, CAS number: 1956329-03-5, CAS number: 1990457-52-7, CAS number: 1990457-55-0, CAS number: 1990457-57-2, CAS number: 1990457-66-3, CAS number: 1990457-77-6, CAS number: 1990457-85-6, CAS number: 2032403-97-5, CAS number: 2044701-44-0, CAS number: 2095470-94-1, CAS Number: 2128706-04-5, CAS number: 2128706-05-6, CAS number: 2133042-31-4, CAS number: 2133042-44-9, CAS number: 2171099-09-3, CAS number: 2220132-55-6, CAS number: 2396747-83-2, CAS number: 2408220-91-5, CAS number: 2408220-94-8, CAS number: 2415706-16-8, Piperflanilide (CAS number: 2615135-05-0), CAS number: 2719848-60-7, CAS number: RNA (Leptinotarsa decemlineata-specific recombinant double-stranded interfering GS2), chlorantraniliprole, chlordane, chlorfenapyr, chloropicrin, chloroprallethrin, chlorpyrifos, chromafenozide, Chrysoperla carnea, clenpirin, cloethocarb, clothianidin, codlelure, codlemone, copper acetoarsenite, copper dioctanoate, copper hydroxide, copper sulfate, cresol, crufomate, Cryptolaemus montrouzieri, cuelure, cyanofenphos, cyantraniliprole, cybutryne, cyclaniliprole, cyclobutrifluram, cycloprothrin, cycloxaprid, Cydia pomonella GV, cyenopyrafen, cyetpyrafen, cyflumetofen, cyfluthrin, cyhalodiamide, cylohalothrin, cypermethrin, cyphenothrin, cyproflanilide, cyromazine, cytokinins, Dacnusa sibirica, dazomet, DBCP, DCIP, deltamethrin, diafenthiuron, dialifos, diamidafos, dibrom, dibutyl adipate, dibutyl phthalate, dibutyl succinate, dichlofenthion, dichlone, dichlorophen, dicliphos, dicloromezotiaz, diethyltoluamide, diflubenzuron, Diglyphus isaea, dimatif, dimethoate, dimethyl carbate, dimethyl phthalate, dimpropyridaz, dinactin, dinocap, dinotefuran, dioxabenzofos, dipyrithione, disparlure, D-limonene, dodec-8-en-1-yl acetate, dodec-9-en-1-yl acetate, dodeca-8,10-dien-1-yl acetate, dodicin, dominicalure, doramectin, emamectin, emamectin benzoate, empenthrin, Encarsia formosa, endothal, endrin, eprinomectin, epsilon-momfluorothrin, epsilon-metofluthrin, Eretmocerus eremicus, esfenvalerate, ethion, ethiprole, ethoprophos, ethyl 4-methyloctanoate, ethyl hexanediol, ethylene dibromide, etofenprox, etoxazole, etpyrafen, eugenol, Extract of seaweed and fermentation product derived from melasse, Extract of seaweed and fermentation product derived from melasse comprising urea, Extract of seaweed and fermented plant products, Extract of seaweed and fermented plant products comprising phytohormones, vitamins, EDTA-chelated copper, zinc, and iron, famphur, fenaminosulf, fenamiphos, fenazaquin, fenfluthrin, fenitrothion, fenmezoditiaz, fenobucarb, fenothiocarb, fenoxycarb, fenpropathrin, fenpyrad, fenpyroximate, fensulfothion, fenthion, fentin, fentinacetate, fenvalerate, ferric phosphate, fipronil, flometoquin, flonicamid, fluacrypyrim, fluazaindolizine, fluazuron, flubendiamide, flubenzimine, fluchlordiniliprole, flucitrinate, flucycloxuron, flucythrinate, fluensulfone [318290-98-1], fluensulfone, flufenerim, flufenprox, flufiprole, fluhexafon, flumethrin, fluopyram, flupyradifurone, flupyrimin, flupyroxystrobin, fluralaner, fluvalinate, fluxametamide, formaldehyde, fosthiazate, fosthietan, frontalin, furfural, gamma-cyhalothrin, Gossyplure® (1:1 mixture of the (Z,E) and (Z,Z) isomers of hexadeca-7,11-dien-1-yl-acetate), grandlure, grandlure I, grandlure II, grandlure III, grandlure IV, Granulovirus, guadipyr, GY-81, halfenprox, halofenozide, Harpin, Helicoverpa armigera Nucleopolyhedrovirus, Helicoverpa zea NPV, Helicoverpa zea Nucleopolyhedrovirus, Heliothis punctigera Nucleopolyhedrovirus, Heliothis virescens Nucleopolyhedrovirus, hemel, hempa, heptafluthrin, heterophos, Heterorhabditis bacteriophora and H. megidis, hexalure, hexamide, hexythiazox, Hippodamia convergens, hydramethylnon, hydrargaphen, hydrated lime, imicyafos, imidacloprid, imiprothrin, Indazapyroxamet, indoxacarb, iodomethane, iprodione, ipsdienol, ipsenol, isamidofos, isazofos, isocycloseram, Isoflualanam (CAS number: 2892524-05-7), isothioate, ivermectin, japonilure, kappa-bifenthrin, kappa-tefluthrin, kasugamycin, kasugamycin hydrochloride hydrate, kinetin, lambda-cyhalothrin, ledprona, lepimectin, Leptomastix dactylopii, lineatin, litlure, looplure, lotilaner, lufenuron, Macrolophus caliginosus, Mamestra brassicae NPV, mecarphon, medlure, megatomoic acid, metaflumizone, metaldehyde, metam, metam-potassium, metam-sodium, Metaphycus helvolus, Metarhizium anisopliae var. acridum, Metarhizium anisopliae var. anisopliae, Metarhizium spp., metepa, methiocarb, methiotepa, methomyl, methoquin-butyl, methoxyfenozide, methyl apholate, methyl bromide, methyl eugenol, methyl isothiocyanate, methylneodecanamide, metofluthrin, metolcarb, mexacarbate, milbemectin, milbemycin oxime, momfluorothrin, morzid, moxidectin, muscalure, Muscodor albus 620 (NRRL Accession No. 30547), Muscodor roseus A 3-5 (NRRL Accession No. 30548), Myrothecium verrucaria composition, nabam, NC-184, Neem tree based products, Neodiprion sertifer NPV and N. lecontei NPV, nickel bis(dimethyldithiocarbamate), niclosamide, niclosamide-olamine, nicofluprole, nitenpyram, nithiazine, nitrapyrin, octadeca-2,13-dien-1-yl acetate, octadeca-3,13-dien-1-yl acetate, octhilinone, omethoate, orfralure, Orius spp., oryctalure, ostramone, oxamate, oxamyl, oxazosulfyl, oxolinic acid, oxytetracycline, Paecilomyces fumosoroseus, Paecilomyces lilacinus, parathion-ethyl, Pasteuria nishizawae, Pasteuria penetrans, Pasteuria ramosa, Pasteuria thornei, Pasteuria usgae, P-cymene, penfluron, pentachlorophenol, permethrin, phenothrin, phorate, phosphamidon, phosphocarb, Phytoseiulus persimilis, picaridin, pioxaniliprole, piperazine, piperonylbutoxide, pirimicarb, pirimiphos-ethyl, pirimiphos-methyl, Plutella xylostella Granulosis virus, Plutella xylostella Nucleopolyhedrovirus, Polyhedrosis virus, potassium and molybdenum and EDTA-chelated manganese, potassium ethylxanthate, potassium hydroxyquinoline sulfate, prallethrin, probenazole, profenofos, profluthrin, propargite, propetamphos, propoxur, prothiophos, protrifenbute, pyflubumide, pymetrozine, pyraclofos, pyrafluprole, pyrethrum, pyridaben, pyridalyl, pyridin-4-amine, pyrifluquinazon, pyrimidifen, pyriminostrobin, pyriprole [394730-71-3], pyriprole, pyriproxyfen, QRD 420 (a terpenoid blend), QRD 452 (a terpenoid blend), QRD 460 (a terpenoid blend), Quillaja saponaria, quinoclamine, quinonamid, resmethrin, Rhodococcus globerulus AQ719 (NRRL Accession No B-21663), sarolaner, S-bioallethrin, sebufos, selamectin, siglure, silafluofen, simazine, sodium pentachlorophenoxide, sordidin, spidoxamat, spinetoram, spinosad, spirobudifen, spirodiclofen, spiromesifen, spiropidion, spirotetramat, Spodoptera exigua multicapsid nuclear polyhedrosis virus, Spodoptera frugiperda Nucleopolyhedrovirus, Steinernema bibionis, Steinernema carpocapsae, Steinernema feltiae, Steinernema glaseri, Steinernema riobrave, Steinernema riobravis, Steinernema scapterisci, Steinernema spp., Streptomyces galbus (NRRL Accession No. 30232), Streptomyces sp. (NRRL Accession No. B-30145), streptomycin, streptomycin sesquisulfate, strychnine, sulcatol, sulfiflumin (CAS number: 2377084-09-6), sulfoxaflor, tazimcarb, tebufenozide, tebufenpyrad, tebupirimiphos, tecloftalam, tefluthrin, temephos, tepa, terbam, terbufos, terpenoid blend, tetrachlorantraniliprole, tetrachlorothiophene, tetradec-11-en-1-yl acetate, tetradiphon, tetramethrin, tetramethylfluthrin, tetranactin, tetraniliprole, theta-cypermethrin, thiacloprid, thiafenox, thiamethoxam, thiocyclam, thiodicarb, thiofanox, thiohempa, thiomersal, thiometon, thionazin, thiophanate, thiosultap, thiotepa, tigolaner, tiorantraniliprole, tioxazafen, tolfenpyrad, toxaphene, tralomethrin, transfluthrin, tretamine, triazamate, triazophos, triazuron, tributyltin oxide, trichlorfon, trichloronate, trichlorphon, Trichogramma spp., trifenmorph, trifluenfuronate, triflumezopyrim, trimedlure, trimedlure A, trimedlure B 1, trimedlure B 2, trimedlure C, trimethacarb, triphenyltin acetate, triphenyltin hydroxide, trunc-call, tyclopyrazoflor, Typhlodromus occidentalis, uredepa, Verticillium lecanii, Verticillium spp., xylenols, YI-5302, zeatin, zeta-Cypermethrin; N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427), (3′,4′,5′-trifluoro-biphenyl-2-yl)-amide, (3-methylisoxazol-5-yl)-[4-[5-(trifluoromethyl)-1,2,4-oxadiazol-3-yl]phenyl]methanone (these compounds may be prepared from the methods described in WO 2017/220485), (4-phenoxyphenyl)methyl 2-amino-6-methyl-pyridine-3-carboxylate (this compound may be prepared from the methods described in WO 2014/006945), (5-methyl-2-pyridyl)-[4-[5-(trifluoromethyl)-1,2,4-oxadiazol-3-yl]phenyl]methanone, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, (E)-6-methylhept-2-en-4-ol, (E)-dec-5-en-1-yl acetate with (E)-dec-5-en-1-ol, (E)-tridec-4-en-1-yl acetate, (E,Z)-tetradeca-4,10-dien-1-yl acetate,, (R)-3-(difluoromethyl)-1-methyl-N-[1,1,3-trimethylindan-4-yl]pyrazole-4-carboxamide, (Z)-dodec-7-en-1-yl acetate, (Z)-hexadec-11-en-1-yl acetate, (Z)-hexadec-11-enal, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (Z,2E)-5-[1-(2,4-dichlorophenyl) pyrazol-3-yl]oxy-2-methoxyimino-N,3-dimethyl-pent-3-enamide (this compound may be prepared from the methods described in WO 2018/153707), (Z,2E)-5-[1-(4-chlorophenyl) pyrazol-3-yl]oxy-2-methoxyimino-N,3-dimethyl-pent-3-enamide,, [2-[3-[2-[1-[2-[3,5-bis(difluoromethyl) pyrazol-1-yl]acetyl]-4-piperidyl]thiazol-4-yl]-4,5-dihydroisoxazol-5-yl]-3-chloro-phenyl]methanesulfonate, 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO2017/025510), 1,1-bis(4-chloro-phenyl)-2-ethoxyethanol, 1,1-dichloro-2,2-bis(4-ethylphenyl)-ethane, 1,2-dibromo-3-chloropropane, 1,2-dichloropropane with 1,3-dichloropropene, 1,3-dichloropropene, 1,3-dimethoxy-1-[[4-[5-(trifluoromethyl)-1,2,4-oxadiazol-3-yl]phenyl]methyl]urea, 1-[2-[1-(4-chlorophenyl) pyrazol-3-yl]oxymethyl]-3-methyl-phenyl]-4-methyl-tetrazol-5-one, 10-dien-1-yl acetate, 14-methyloctadec-1-ene, 1-bromo-2-chloroethane, 1-dichloro-1-nitroethane, 1-hydroxy-1H-pyridine-2-thione, 1-methoxy-3-methyl-1-[4-[5-(trifluoromethyl)-1,2,4-oxadiazol-3-yl]phenyl]methyl]urea, 1-methyl-4-[3-methyl-2-[[2-methyl-4-(3,4,5-trimethylpyrazol-1-yl)phenoxy]methyl]phenyl]tetrazol-5-one, 2-(difluoromethyl)-N-((3R)-1, 1, 3-trimethylindan-4-yl)pyridine-3-carboxamide, 2-(difluoromethyl)-N-((3R)-1, 1, 3-trimethylindan-4-yl)pyridine-3-carboxamide, 2-(1,3-dithiolan-2-yl)phenyl dimethylcarbamate, 2-(2-butoxyethoxy)-ethyl piperonylate, 2-(2-butoxyethoxy)ethyl thiocyanate, 2-(4,5-dimethyl-1,3-dioxolan-2-yl)phenyl methylcarbamate, 2-(4-chloro-3,5-xylyloxy) ethanol, 2-(difluoromethyl)-N-(3-ethyl-1,1-dimethyl-indan-4-yl)pyridine-3-carboxamide, 2-(difluoromethyl)-N-[(3R)-3-ethyl-1,1-dimethyl-indan-4-yl]pyridine-3-carboxamide, 2-(difluoromethyl)-N-[(3S)-3-ethyl-1,1-dimethyl-indan-4-yl]pyridine-3-carboxamide (this compound may be prepared from the methods described in WO 2014/095675), 2-(difluoromethyl)-N-[3-ethyl-1,1-dimethyl-indan-4-yl]pyridine-3-carboxamide, 2-(octylthio)-ethanol, 2,2,2-trichloro-1-(3,4-dichloro-phenyl)ethyl acetate, 2,2-dichlorovinyl 2-ethylsulfinylethyl methyl phosphate, 2,2-difluoro-N-methyl-2-[4-[5-(trifluoromethyl)-1,2,4-oxadiazol-3-yl]phenyl]acetamide, 2,4-dichlorophenyl benzenesulfonate, 2,6-Dimethyl-1H,5H-[1,4]dithiino[2,3-c:5,6-c′]dipyrrole-1,3,5,7 (2H,6H)-tetrone (this compound may be prepared from the methods described in WO 2011/138281), 2-[2-fluoro-6-[(8-fluoro-2-methyl-3-quinolyl)oxy]phenyl]propan-2-ol, 2-[6-(4-bromophenoxy)-2-(trifluoromethyl)-3-pyridyl]-1-(1,2,4-triazol-1-yl) propan-2-ol (this compound may be prepared from the methods described in WO 2017/029179), 2-[6-(4-chlorophenoxy)-2-(trifluoromethyl)-3-pyridyl]-1-(1,2,4-triazol-1-yl) propan-2-ol (this compound may be prepared from the methods described in WO 2017/029179), 2-chlorovinyl diethyl phosphate, 2-fluoro-N-methyl-N-1-naphthylacetamide, 2-imidazolidone, 2-isovalerylindan-1,3-dione, 2-methyl (prop-2-ynyl)aminophenyl methylcarbamate, 2-oxo-N-propyl-2-[4-[5-(trifluoromethyl)-1,2,4-oxadiazol-3-yl]phenyl]acetamide (this compound may be prepared from the methods described in WO 2018/065414), 2-thiocyanatoethyl laurate, 3-(4,4-difluoro-3,3-dimethyl-1-isoquinolyl)-7,8-dihydro-6H-cyclopenta[e]benzimidazole (these compounds may be prepared from the methods described in WO2016/156085), 3-(4,4-difluoro-3,4-dihydro-3,3-dimethylisoquinolin-1-yl) quinolone, 3-(4-chlorophenyl)-5-methylrhodanine, 3-(difluoromethyl)-1-methyl-N-[1,1,3-trimethylindan-4-yl]pyrazole-4-carboxamide, 3,4-dichlorotetrahydrothio-phene 1,1-dioxide, 3-[2-(1-chlorocyclopropyl)-3-(2-fluorophenyl)-2-hydroxy-propyl]imidazole-4-carbonitrile (this compound may be prepared from the methods described in WO 2016/156290), 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290), 3-bromo-1-chloroprop-1-ene, 3-chloro-6-methyl-5-phenyl-4-(2,4,6-trifluorophenyl)pyridazine, 3-difluoromethyl-1-methyl-1H-pyrazole-4-carboxylic acid, 3-ethyl-1-methoxy-1-[[4-[5-(trifluoromethyl)-1,2,4-oxadiazol-3-yl]phenyl]methyl]urea, 3-methyl-1-phenylpyrazol-5-yl dimethyl-carbamate, 4-(2-bromo-4-fluorophenyl)-N-(2-chloro-6-fluorophenyl)-1, 3-dimethyl-1H-pyrazol-5-amine, 4-(2,6-difluorophenyl)-6-methyl-5-phenyl-pyridazine-3-carbonitrile, 4-(2-bromo-4-fluoro-phenyl)-N-(2-chloro-6-fluoro-phenyl)-2,5-dimethyl-pyrazol-3-amine, 4-(quinoxalin-2-ylamino)benzenesulfonamide, 4,4-difluoro-1-(5-fluoro-4-methyl-benzimidazol-1-yl)-3,3-dimethyl-isoquinoline, 4,4-difluoro-3,3-dimethyl-1-(6-methylpyrazolo[1,5-a]pyridin-3-yl) isoquinoline, 4,4-difluoro-3,3-dimethyl-1-(7-methylpyrazolo[1,5-a]pyridin-3-yl) isoquinoline, 4,4-dimethyl-2-[[4-[5-(trifluoromethyl)-1,2,4-oxadiazol-3-yl]phenyl]methyl]isoxazolidin-3-one, 4-[6-[2-(2,4-difluorophenyl)-1,1-difluoro-2-hydroxy-3-(1,2,4-triazol-1-yl) propyl]-3-pyridyl]oxy]benzonitrile, 4-[6-[2-(2,4-difluorophenyl)-1,1-difluoro-2-hydroxy-3-(5-sulfanyl-1,2,4-triazol-1-yl) propyl]-3-pyridyl]oxy]benzonitrile, 4-[6-[2-(2,4-difluorophenyl)-1,1-difluoro-2-hydroxy-3-(5-thioxo-4H-1,2,4-triazol-1-yl) propyl]-3-pyridylloxy]benzonitrile, 4-chloro-2-(2-chloro-2-methyl-propyl)-5-[(6-iodo-3-pyridyl) methoxylpyridazin-3-one, 4-chlorophenyl phenyl sulfone, 4-methyl (prop-2-ynyl)amino-3,5-xylyl methylcarbamate, 4-methylnonan-5-ol with 4-methylnonan-5-one, 5-(1,3-benzodioxol-5-yl)-3-hexylcyclohex-2-enone, 5,5-dimethyl-2-[4-[5-(trifluoromethyl)-1,2,4-oxadiazol-3-yl]phenyl]methyl]isoxazolidin-3-one, 5,5-dimethyl-3-oxocyclohex-1-enyl dimethylcarbamate, 5-amino-1,3,4-thiadiazole-2-thiol zinc salt (2:1), 5-methyl-6-thioxo-1,3,5-thiadiazinan-3-ylacetic acid, 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391), 6-chloro-4,4-difluoro-3,3-dimethyl-1-(4-methylbenzimidazol-1-yl) isoquinoline, 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391), 6-ethyl-5,7-dioxo-pyrrolo [4,5][1,4]dithiino[1,2-c]isothiazole-3-carbonitrile, 6-isopentenylaminopurine, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, 8-hydroxyquinoline sulfate, acethion, acetoprole, acibenzolar, acibenzolar-S-methyl, acrylonitrile, Adoxophyes orana GV, Agrobacterium radiobacter, aldoxycarb, aldrin, allosamidin, allyxycarb, alpha-chlorohydrin, alpha-ecdysone, alpha-multistriatin, aluminium phosphide, Amblyseius spp., amectotractin, ametoctradin, amidithion, amidothioate, aminocarb, aminopyrifen, amisulbrom, amiton, amiton hydrogen oxalate, amitraz, anabasine, Anagrapha falcifera NPV, Anagrus atomus, ancymidol, anilazine, anisiflupurin, anthraquinone, antu, Aphelinus abdominalis, Aphidius colemani, Aphidoletes aphidimyza, apholate, aramite, arsenous oxide, athidathion, Autographa californica NPV, azaconazole, azamethiphos, azobenzene, azothoate, azoxystrobin, Bacillus sphaericus Neide, Bacillus thuringiensis delta endotoxins, barium carbonate, barium hexafluorosilicate, barium polysulfide, barthrin, Bayer 22/190, Bayer 22408, Beauveria brongniartii, benalaxyl, benclothiaz, benomyl, benoxa-fos, benthiavalicarb, benzothiostrobin, benzovindiflupyr, benzyl benzoate, beta-cyfluthrin, beta-cypermethrin, bethoxazin, bioethanomethrin, biopermethrin, bis(2-chloroethyl) ether, bis (tributyltin) oxide, bisazir, bisthiosemi, bitertanol, bixafen, blasticidin-S, borax, bordeaux mixture, boscalid, brevicomin, brodifacoum, brofenvalerate, bromadiolone, bromethalin, bromfenvinfos, bromoacetamide, bromo-cyclen, bromo-DDT, bromophos, bromopropylate, bromuconazole, bronopol, bufencarb, bupirimate, buprofezin, busulfan, but-3-ynyl N-[6-[[(Z)-[(1-methyltetrazol-5-yl)-phenyl-methylene]amino]oxymethyl]-2-pyridyl]carbamate, butacarb, butathiofos, butocarboxim, butonate, butopyronoxyl, butoxy (polypropylene glycol), butoxycarboxim, butylpyridaben, calcium arsenate, calcium cyanide, calcium polysulfide, camphechlor, captafol, captan, carbanolate, carbendazim, carbon disulfide, carbon tetrachloride, carbophenothion, carboxin, cartap hydrochloride, CAS Number: 2132414-04-9, CAS Number: 2344721-61-3, cevadine, chino-methionat, chloralose, chlorbenside, chlorbicyclen, chlordane, chlordecone, chlordimeform, chlordimeform hydrochloride, chlorfenethol, chlorfenson, chlorfensulfide, chlorobenzilate, chloroform, chloroinconazide, chloromebuform, chloromethiuron, chloroneb, chlorophacinone, chloropicrin, chloropropylate, chloro¬tha¬lo¬nil, chlorphoxim, chlorprazophos, chlorthiophos, chlozolinate, cholecalciferol, Chrysoperla carnea, cinerin I, cinerin II, cinerins, cismethrin, cis-resmethrin, clocythrin, closantel, codlelure, codlemone, copper acetoarsenite, copper arsenate, copper dioctanoate, copper hydroxide, copper naphthenate, copper oleate, copper oxide, copper oxychloride, copper sulfate, coumachlor, coumafuryl, coumaphos, coumatetralyl, coumethoxystrobin (jiaxiangjunzhi), coumithoate, coumoxystrobin, cresol, crimidine, crotamiton, crotoxyphos, crufomate, cryolite, Cryptolaemus montrouzieri, CS 708, cuelure, cufraneb, cyanofenphos, cyanophos, cyanthoate, cyazofamid, cybutryne, cyclethrin, cyclobutrifluram, Cydia pomonella GV, cyflufenamid, cymiazole, cymoxanil, cyproconazole, cyprodinil, cythioate, cytokinins, Dacnusa sibirica, DAEP, dazomet, DCIP, DCPM, DDT, debacarb, decarbofuran, demephion, demephion-O, demephion-S, demeton-methyl, demeton-0, demeton-O-methyl, demeton-S, demeton-S-methyl, demeton-S-methylsulfon, diamidafos, dibutyl adipate, dibutyl phthalate, dibutyl succinate, dicapthon, dichlobentiazox, dichlofenthion, dichlofluanid, dichlone, dichlorophen, dichlorvos, dichlozoline, dicliphos, diclocymet, diclomezine, dicloran, dicresyl, dicyclanil, dicyclopentadiene, dieldrin, dienochlor, diethofencarb, diethyl 5-methylpyrazol-3-yl phosphate, diethyltoluamide, difenacoum, difenoconazole, difethialone, diflovidazin, Diglyphus isaea, dilor, dimatif, dimefluthrin, dimefox, dimetan, dimethirimol, dimetho-morph, dimethrin, dimethyl carbate, dimethyl phthalate, dimethylvinphos, dimetilan, dimoxystrobin, dinex, dinex-diclexine, diniconazole, dinocap-4, dinocap-6, dinocton, dino-penton, dinoprop, dinosam, dinoseb, dinosulfon, dinoterbon, diofenolan, dioxabenzofos, dioxathion, diphacinone, diphenyl sulfone, dipymetitrone, dipyrithione, disparlure, disulfiram, dithianon, dithicrofos, DNOC, dodec-8-en-1-yl acetate, dodec-9-en-1-yl acetate, dodeca-8, dodemorph, dodicin, dodine, dofenapyn, dominicalure, doramectin, DSP, d-tetramethrin, ecdysterone, edifenphos, EI 1642, EMPC, Encarsia formosa, endothal, endothion, enestroburin, enoxastrobin, EPBP, epoxicon-azole, eprinomectin, Eretmocerus eremicus, ergocalciferol, etaphos, ethaboxam, ethiofencarb, ethirimol, ethoate-methyl, ethyl 1-[[4-[(Z)-2-ethoxy-3,3,3-trifluoro-prop-1-enoxy]phenyl]methyl]pyrazole-3-carboxylate (may be prepared from the methods described in WO 2020/056090), ethyl 1-[[4-[[2-(trifluoromethyl)-1,3-dioxolan-2-yl]methoxy]phenyl]methyl]pyrazole-3-carboxylate (may be prepared from the methods described in WO 2020/056090), ethyl 1-[[4-[5-(trifluoromethyl)-1,2,4-oxadiazol-3-yl]phenyl]methyl]pyrazole-4-carboxylate, ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365), ethyl 4-methyloctanoate, ethyl formate, ethyl hexanediol, ethylene dibromide, ethylene dichloride, ethylene oxide, etridiazole, etrimfos, eugenol, EXD, famoxa-done, farnesol, farnesol with nerolidol, fenamidone, fenaminosulf, fenaminstrobin, fenarimol, fenazaflor, fenbuconazole, fenbutatin oxide, fenchlorphos, fenethacarb, fenfuram, fenhexamid, fenitrothion, fenothiocarb, fenoxacrim, fenoxanil, fenpiclonil, fenpicoxamid, fenpirithrin, fenpropidin, fenpropimorph, fenpyrad, fenpyrazamine, fen-pyroximate, fenson, fensulfothion, fenthion, fenthion-ethyl, fentin, fentrifanil, ferbam, ferimzone, ferric phosphate, flocoumafen, florylpicoxamid, fluazinam, flubeneteram, flubenzimine, flucofuron, flucycloxuron, fludioxonil, fluenetil, flufenoxadiazam, flufenoxystrobin, fluindapyr, flumetylsulforim, flumorph, fluopicolide, fluopimomide, fluopyram, fluorbenside, fluoroacetamide, fluoroimide, fluoxapiprolin, fluoxastrobin, fluoxytioconazole, flupropadine, flupropadine hydrochloride, fluquinconazole, flusilazole, flusulfamide, flutianil, flutolanil, flutriafol, fluxapyroxad, FMC 1137, folpet, formaldehyde, formetanate, formetanate hydrochloride, formparanate, fosetyl-aluminium, fosmethilan, fospirate, fosthietan, frontalin, fuberidazole, furalaxyl, furametpyr, furathiocarb, furethrin, furfural, gamma-HCH, glyodin, grandlure, grandlure I, grandlure II, grandlure III, grandlure IV, guazatine, guazatine acetates, halfenprox, HCH, hemel, hempa, HEOD, heptachlor, heterophos, Heterorhabditis bacteriophora and H. megidis, hexaconazole, hexadecyl cyclopropanecarboxylate, hexalure, hexamide, HHDN, Hippodamia convergens, hydrargaphen, hydrated lime, hydrogen cyanide, hymexazol, hyquincarb, imanin, imazalil, imiben¬con¬azole, iminoctadine, inpyrfluxam, ipconazole, ipfentrifluconazole, ipflufenoquin, iprobenphos, iprodione, iprovalicarb, ipsdienol, ipsenol, IPSP, isamidofos, isazofos, isobenzan, isocarbophos, isodrin, isofenphos, isofetamid, isoflucypram, isolane, isoprothiolane, isopyrazam, isotianil, isoxathion, japonilure, jasmolin I, jasmolin II, jodfenphos, juvenile hormone I, juvenile hormone II, juvenile hormone III, kadethrin, kasugamycin, kasugamycin hydrochloride hydrate, kelevan, kinetin, kinoprene, kresoxim-methyl, lead arsenate, Leptomastix dactylopii, leptophos, lindane, lineatin, lirimfos, litlure, looplure, Ivbenmixianan, lythidathion, Macrolophus caliginosus, magnesium phosphide, malonoben, Mamestra brassicae NPV, mancopper, mancozeb, mandestrobin, mandipropamid, maneb, mazidox, m-cumenyl methylcarbamate, mecarbam, mecarphon, medlure, mefentrifluconazole, megatomoic acid, menazon, mepanipyrim, meperfluthrin, mephosfolan, mepronil, mercuric oxide, mercurous chloride, mesulfen, mesulfenfos, meta-laxyl, metam, metam-potassium, metam-sodium, Metaphycus helvolus, Metarhizium anisopliae var. acridum, Metarhizium anisopliae var. anisopliae, metarylpicoxamid, metconazole, metepa, methacrifos, methanesulfonyl fluoride, methasulfo-carb, methiotepa, methocrotophos, methoprene, methoquin-butyl, methothrin, methoxychlor, methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-(4-propyltriazol-2-yl)phenoxy]prop-2-enoate, methyl (Z)-3-methoxy-2-[2-methyl-5-[3-(trifluoromethyl) pyrazol-1-yllphenoxy]prop-2-enoate (these compounds may be prepared from the methods described in WO2020/079111), methyl (Z)-3-methoxy-2-[2-methyl-5-[4-(trifluoromethyl)triazol-2-yllphenoxy]prop-2-enoate, methyl apholate, methyl bromide, methyl eugenol, methyl isothiocyanate, methyl N-[[4-[1-(2,6-difluoro-4-isopropyl-phenyl) pyrazol-4-yl]-2-methyl-phenyl]methyl]carbamate (may be prepared from the methods described in WO 2020/097012), methyl N-[4-[1-(4-cyclopropyl-2,6-difluoro-phenyl) pyrazol-4-yl]-2-methyl-phenyl]methyl]carbamate (may be prepared from the methods described in WO 2020/097012), methyl N-[5-[4-(2,4-dimethylphenyl)triazol-2-yl]-2-methyl-phenyl]methyl]carbamate, methylchloroform, methylene chloride, methylneodecanamide, metiram, metolcarb, metominostrobin, metoxadiazone, metrafenone, metyltetraprole, MGK 264, milbemycin oxime, mipafox, mirex, monocrotophos, morphothion, morzid, moxidectin, muscalure, myclobutanil, myclozoline, Myrothecium verrucaria composition, N-((1R)-1-benzyl-3-chloro-1-methyl-but-3-enyl)-8-fluoro-quinoline-3-carboxamide (these compounds may be prepared from the methods described in WO2017/153380), N-((1S)-1-benzyl-3-chloro-1-methyl-but-3-enyl)-8-fluoro-quinoline-3-carboxamide (these compounds may be prepared from the methods described in WO2017/153380), N′-(2,5-dimethyl-4-phenoxy-phenyl)-N-ethyl-N-methyl-formamidine, N′-(2-chloro-5-methyl-4-phenoxy-phenyl)-N-ethyl-N-methyl-formamidine, N,2-dimethoxy-N-[4-[5-(trifluoromethyl)-1,2,4-oxadiazol-3-yl]phenyl]methyl]propanamide, N,N-dimethyl-1-[4-[5-(trifluoromethyl)-1,2,4-oxadiazol-3-yl]phenyl]methyl]-1,2,4-triazol-3-amine (THESE COM POUNDS may be prepared from the methods described in WO 2017/055473, WO 2017/055469, WO 2017/093348 and WO 2017/118689), N-[(1R)-1-benzyl-1,3-dimethyl-butyl]-7,8-difluoro-quinoline-3-carboxamide, N-[(1R)-1-benzyl-1,3-dimethyl-butyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-1,3-dimethyl-butyl]-7,8-difluoro-quinoline-3-carboxamide, N-[(1S)-1-benzyl-1,3-dimethyl-butyl]-8-fluoro-quinoline-3-carboxamide, N-[(1S)-1-benzyl-3,3,3-trifluoro-1-methyl-propyl]-8-fluoro-quinoline-3-carboxamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, N-[[4-[5-(trifluoromethyl)-1,2,4-oxadiazol-3-yl]phenyl]methyl]propanamide, N-[2-[2,4-dichloro-phenoxy]phenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, N′-[2-chloro-4-(2-fluorophenoxy)-5-methyl-phenyl]-N-ethyl-N-methyl-formamidine (this compound may be prepared from the methods described in WO 2016/202742), N′-[4-(4,5-dichlorothiazol-2-yl)oxy-2,5-dimethyl-phenyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075), N′-[5-bromo-2-methyl-6-(2-propoxypropoxy)-3-pyridyl]-N-ethyl-N-methyl-formamidine (this compound may be prepared from the methods described in IPCOM 000249876D), N′-[5-bromo-2-methyl-6-[(1R)-1-methyl-2-propoxy-ethoxy]-3-pyridyl]-N-ethyl-N-methyl-formamidine, N′-[5-bromo-2-methyl-6-[(1S)-1-methyl-2-propoxy-ethoxy]-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N-[N-methoxy-C-methyl-carbonimidoyl]-4-[5-(trifluoromethyl)-1,2,4-oxadiazol-3-yl]benzamide (these compounds may be prepared from the methods described in WO 2018/202428), N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896), nabam, naftalofos, naled, naphthalene, NC-170, Neodiprion sertifer NPV and N. lecontei NPV, nerolidol, N-ethyl-2-methyl-N-[[4-[5-(trifluoromethyl)-1,2,4-oxadiazol-3-yl]phenyl]methyl]propanamide, N-ethyl-N′-[5-methoxy-2-methyl-4-[(2-trifluoromethyl) oxetan-2-yl]phenyl]-N-methyl-formamidine, nickel bis(dimethyldithiocarbamate), niclosamide-olamine, nicotine, nicotine sulfate, nifluridide, nikkomycins, N-isopropyl-N′-[5-methoxy-2-methyl-4-(2,2,2-trifluoro-1-hydroxy-1-phenyl-ethyl)phenyl]-N-methyl-formamidine, nithiazine, nitrapyrin, nitrilacarb, nitrilacarb 1:1 zinc chloride complex, nitrothal-isopropyl, N-methoxy-N-[[4-[5-(trifluoromethyl)-1,2,4-oxadiazol-3-yl]phenyl]methyl]cyclopropanecarboxamide, N-methyl-4-[5-(trifluoromethyl)-1,2,4-oxadiazol-3-yl]benzamide, N-methyl-4-[5-(trifluoromethyl)-1,2,4-oxadiazol-3-yl]benzenecarbothioamide, norbormide, nuarimol, 0,0,0′,O′-tetrapropyl dithiopyrophosphate, octadeca-2,13-dien-1-yl acetate, octadeca-3,13-dien-1-yl acetate, octhilinone, ofurace, oleic acid, omethoate, orfralure, Orius spp., oryctalure, orysastrobin, ostramone, oxadixyl, oxamate, oxathiapiprolin, oxine-copper, oxolinic acid, oxycarboxin, oxydeprofos, oxydisulfoton, oxytetracycline, paclobutrazole, Paecilomyces fumosoroseus, para-dichlorobenzene, parathion, parathion-methyl, pefurazoate, penconazole, pencycuron, penflufen, penfluron, pentachlorophenol, pentachlorophenyl laurate, penthiopyrad, permethrin, PH 60-38, phenamacril, phenkapton, phosacetim, phosalone, phosdiphen, phosfolan, phosglycin, phosnichlor, phosphamidon, phosphine, phosphorus, phoxim-methyl, phthalide, Phytoseiulus persimilis, picarbutrazox, picaridin, picoxystrobin, pindone, piperazine, piperonyl butoxide, piprotal, pirimetaphos, polychlorodicyclopentadiene isomers, polychloroterpenes, polynactins, polyoxins, potassium arsenite, potassium ethylxanthate, potassium hydroxyquinoline sulfate, potassium thiocyanate, pp′-DDT, precocene I, precocene II, precocene III, primidophos, probenazole, prochloraz, proclonol, procymi¬done, profluthrin, promacyl, promecarb, propamocarb, propiconazole, propineb, propoxur, propyl isomer, proquinazid, prothidathion, prothioconazole, prothiofos, prothoate, pydiflumetofen, pyraclostrobin, pyrametostrobin, pyraoxystrobin, pyrapropoyne, pyraziflumid, pyrazophos, pyresmethrin, pyrethrin I, pyrethrin II, pyrethrins, pyribencarb, pyridachlometyl, pyridaphenthion, pyridin-4-amine, pyrifenox, pyrimethanil, pyrimitate, pyrimorph, pyrinuron, pyriofenone, pyrisoxazole, pyroquilon, quassia, quinalphos, quinalphos-methyl, quinoclamine, quinofumelin, quinonamid, quinothion, quinoxyfen, quintiofos, quintozene, R-1492, rafoxanide, resmethrin, Reynoutria sachalinensis extract, ribavirin, R metalaxyl, rotenone, ryania, ryanodine, S421, sabadilla, schradan, scilliroside, seboctylamine, sebufos, sedaxane, selamectin, sesamex, sesasmolin, SI-0009, siglure, simazine, simeconazole, sodium arsenite, sodium cyanide, sodium fluoride, sodium fluoro-acetate, sodium hexafluorosilicate, sodium pentachlorophenoxide, sodium selenate, sodium tetrathiocarbonate, sodium thiocyanate, sophamide, sordidin, spiroxamine, SSI-121, Steinernema bibionis, Steinernema carpocapsae, Steinernema feltiae, Steinernema glaseri, Steinernema riobrave, Steinernema riobravis, Steinernema scapterisci, Steinernema spp., streptomycin, streptomycin sesquisulfate, strychnine, sulcatol, sulcofuron, sulcofuron-sodium, sulfiram, sulfluramid, sulfotep, sulfoxide, sulfur, sulfuryl fluoride, sulprofos, tar oils, tau-fluvalinate, tazimcarb, TDE, tebuconazole, tebufloquin, tebupirimfos, tecloftalam, temephos, tepa, TEPP, terallethrin, terbam, tert-butyl N-[6-[[[(1-methyltetrazol-5-yl)-phenyl-methylene]amino]oxymethyl]-2-pyridyl]carbamate, tetrachloroethane, tetrachlorothiophene, tetraconazole, tetradec-11-en-1-yl acetate, tetradifon, tetramethylfluthrin, tetrasul, thallium sulfate, thiaben¬dazole, thiafenox, thiapronil, thicrofos, thifluzamide, thiocarboxime, thiocyclam, thiocyclam hydrogen oxalate, thiodiazole copper, thiofanox, thiohempa, thiomersal, thiometon, thionazin, thiophanate, thiophanate-methyl, thioquinox, thiosultap, thiosultap-sodium, thiotepa, thiram, thuringiensin, tiadinil, tolclofos-methyl, tolprocarb, tolylfluanid, tralomethrin, transpermethrin, tretamine, triadimefon, triadime¬nol, triamiphos, triarathene, triazamate, triazophos, triazoxide, triazuron, tributyltin oxide, trichlormetaphos-3, trichloronat, Trichogramma spp., triclopyricarb, tricyclazole, tridemorph, trifenmorph, trifenofos, trifloxystrobin, triflumizole, triforine, trimedlure, trimedlure A, trimedlure B1, trimedlure B2, trimedlure C, trimethacarb, trinactin, trinexapac, triphenyltin acetate, triphenyltin hydroxide, triprene, triticonazole, trunc-call, Typhlodromus occidentalis, uredepa, validamycin, valifenalate, vamidothion, vaniliprole, veratridine, veratrine, verbutin, Verticillium lecanii, vinclozoline, warfarin, XMC, xylenols, zeatin, zetamethrin, zhongshengmycin, zinc naphthenate, zinc phosphide, zinc thiazole, zineb, ziram, zolaprofos; Acinetobacter lwoffii, Acremonium alternatum, Acremonium cephalosporium, Acremonium diospyri, Acremonium obclavatum, Adoxophyes orana granulovirus (AdoxGV) (CAPEX®), Agrobacterium radiobacter strain K 84 (GALLTROL-A®), Alternaria alternate, Alternaria cassia, Alternaria destruens (SMOLDER®), Ampelomyces quisqualis (AQ10®), Aspergillus flavus AF36 (A F36®), Aspergillus flavus NRRL 21882 (AFLAGUARD®), Aspergillus spp., Aureobasidium pullulans, Azospirillum (MICROAZ®, TAZOB®), Azotobacter, Azotobacter chroocuccum (AZOTOMEAL®), Azotobacter cysts (BIONATURAL BLOOMING BLOSSOMS®), Bacillus amyloliquefaciens, Bacillus cereus, Bacillus chitinosporus strain AQ746, Bacillus chitinosporus strain CM-1, Bacillus circulans, Bacillus firmus (BIOSAFE®, BIONEM-WP®) in particular strain CNMC 1-1582 (e.g. VOTIVO® from BASF SE), Bacillus licheniformis strain 3086 (ECOGUARD®, GREEN RELEAF®), Bacillus licheniformis strain HB-2 (RHIZOBOOST®), Bacillus macerans, Bacillus marismortui, Bacillus megaterium, Bacillus mycoides strain AQ726, Bacillus papillae (MILKY SPORE POWDER®), Bacillus pumilus spp., Bacillus pumilus strain AQ717, Bacillus pumilus strain GB 34 (YIELD SHIELD®), Bacillus pumilus strain QST 2808 (SONATA®, BALLAD PLUS®), Bacillus sphaericus (VECTOLEX®), Bacillus spp., Bacillus spp. strain AQ175, Bacillus spp. strain AQ177, Bacillus spp. strain AQ178, Bacillus subtilis strain AQ153, Bacillus subtilis strain AQ743, Bacillus subtilis strain QST 713 (CEASE®, SERENADE®, RHAPSODY®), Bacillus subtilis strain QST 714 (JAZZ®), Bacillus subtilis strain QST 3002, Bacillus subtilis strain QST 3004, Bacillus subtilis var. amyloliquefaciens strain FZB 24 (TAEGRO®, RHIZOPRO®), Bacillus thuringiensis aizawai GC 91 (AGREE®), Bacillus thuringiensis Cry 2Ae, Bacillus thuringiensis Cry1Ab, Bacillus thuringiensis israelensis (BMP123®, AQUABAC®, VECTOBAC®), Bacillus thuringiensis kurstaki (JAVELIN®, DELIVER®, CRY MAX®, BONIDE®, SCUTELLA WP®, TURILAV WP®, ASTUTO®, DIPEL WP®, BIOBIT®, FORAY®), Bacillus thuringiensis kurstaki BMP 123 (BARITONE®), Bacillus thuringiensis kurstaki HD-1 (Bioprotec-CAF/3P®), Bacillus thuringiensis strain AQ52, Bacillus thuringiensis strain BD #32, Bacillus thuringiensis tenebrionis (NOVODOR®, BtBooster), Bacillus thuringiensis var. aizawai (XENTARI®, DIPEL®), bacteria spp. (GROWMEND®, GROWSWEET®, SHOOTUP®), bacteriophage of Clavipacter michiganensis (AGRIPHAGE®, BAKFLOR®), Beauveria bassiana (BEAUGENIC®, BROCARIL WP®), Beauveria bassiana GHA (MYCOTROLES®, MYCOTROLO®, BOTANIGUARD®), Beauveria brongniartii (ENGERLINGSPILZ®, SCHWEIZER BEAUVERIA®, MELOCONT®), Beauveria spp., Botrytis cineria, Bradyrhizobium japonicum (TERRAMAX®), Brevibacillus brevis, Burkholderia cepacia (DENY®, INTERCEPT®, BLUE CIRCLE®), Burkholderia gladii, Burkholderia gladioli, Burkholderia spp., Canadian thistle fungus (CBH CANADIAN BIOHERBICIDE®), Candida butyri, Candida famata, Candida fructus, Candida glabrata, Candida guilliermondii, Candida melibiosica, Candida oleophila strain O, Candida parapsilosis, Candida pelliculosa, Candida pulcherrima, Candida reukaufii, Candida saitoana (BIO-COAT®, BIOCURE®), Candida sake, Candida spp., Candida tenius, Cedecea davisae, Cellulomonas flavigena, Chaetomium cochliodes (NOVA-CIDE®), Chaetomium globosum (NOVA-CIDE®), Chromobacterium subtsugae strain PRAA 4-1T (Grandevo®), Cladosporium chlorocephalum, Cladosporium cladosporioides, Cladosporium oxysporum, Cladosporium spp., Cladosporium tenuissimum, Clonostachys rosea (ENDOFINE®), Colletotrichum acutatum, Coniothyrium minitans (COTANS WG®), Coniothyrium spp., Cryptococcus albidus (YIELDPLUS®), Cryptococcus humicola, Cryptococcus infirmo-miniatus, Cryptococcus laurentii, Cryptophlebia leucotreta granulovirus (CRYPTEX®), Cupriavidus campinensis, Cydia pomonella granulovirus (CYD-X®, MADEX®, MADEX® PLUS, MADEX MAX, CARPOVIRUSINE®), Cylindrobasidium laeve (STUM POUT®), Cylindrocladium, Debaryomyces hansenii, Drechslera hawaiinensis, Enterobacter cloacae, Enterobacteriaceae, Entomophtora virulenta (VEKTOR®), Epicoccum nigrum, Epicoccum purpurascens, Epicoccum spp., Filobasidium floriforme, Fusarium acuminatum, Fusarium chlamydosporum, Fusarium oxysporum (FUSACLEAN®, BIOFOX C®), Fusarium proliferatum, Fusarium spp., Galactomyces geotrichum, Gliocladium catenulatum (PRIMASTOP®, PRESTOP®), Gliocladium roseum, Gliocladium spp. (SOILGARD®), Gliocladium virens (SOILGARD®), Granulovirus (GRANUPOM®), Halobacillus halophilus, Halobacillus litoralis, Halobacillus trueperi, Halomonas spp., Halomonas subglaciescola, Halovibrio variabilis, Hanseniaspora uvarum, Helicoverpa armigera nucleopolyhedrovirus (HELICOVEX®), Helicoverpa zea nuclear polyhedrosis virus (GEMSTAR®), Isaria fumosorosea (previously known as Paecilomyces fumosoroseus strain, PFR-97®, PREFERAL®), Isoflavone formononetin (MYCONATE®), Kloeckera apiculata, Kloeckera spp., Lagenidium giganteum (LAGINEX®), Lecanicillium lecanii (formerly known as Verticillium lecanii (MYCOTAL®) conidia of strain KV 01 (e.g. VERTALEC® by Koppert/Arysta), Lecanicillium longisporum (VERTIBLAST®), Lecanicillium muscarium (VERTIKIL®), Lymantria Dispar nucleopolyhedrosis virus (DISPARVIRUS®), Marinococcus halophilus, Meira geulakonigii, Metarhizium anisopliae (DESTRUXIN WP®), Metarhizium anisopliae (MET52®), Metschnikowia fruticola (SHEMER®), Metschnikowia pulcherrima, Microdochium dimerum (ANTIBOT®), Micromonospora coerulea, Microsphaeropsis ochracea, Muscodor albus 620 (MUSCUDOR®), Muscodor roseus in particular strain A 3-5 (Accession No. NRRL 30548), Mycorrhizae spp. (AMYKOR®, ROOT MAXIMIZER®), Myrothecium verrucaria strain AARC-0255 (DITERA®, BROS PLUS®), Ophiostoma piliferum strain D97 (SYLVANEX®), Paecilomyces farinosus, Paecilomyces lilacinus strain 251 (MELOCON WG®), Paecilomyces linacinus (BIOSTAT WP®), Paenibacillus polymyxa, Pantoea agglomerans (BLIGHTBAN C9-1®), Pantoea spp., Pasteuria nishizawae in particular strain Pn1 (CLARIVA from Syngenta/ChemChina), Pasteuria spp. (ECONEM®), Penicillium aurantiogriseum, Penicillium billai (JUMPSTART®, TAGTEAM®), Penicillium brevicompactum, Penicillium frequentans, Penicillium griseofulvum, Penicillium purpurogenum, Penicillium spp., Penicillium viridicatum, Phlebiopsis gigantean (Rotstop®), phosphate solubilizing bacteria (PHOSPHOMEAL®), Phytophthora cryptogea, Phytophthora palmivora (DEVINE®), Pichia anomala, Pichia guilliermondii, Pichia membranaefaciens, Pichia onychis, Pichia stipites, Pseudomonas aeruginosa, Pseudomonas aureofasciens, Pseudomonas cepacia, Pseudomonas chlororaphis (ATEZE®), Pseudomonas corrugate, Pseudomonas fluorescens (ZEQUA NOX®), Pseudomonas fluorescens strain A 506 (BLIGHTBAN A 506®), Pseudomonas putida, Pseudomonas reactans, Pseudomonas spp., Pseudomonas syringae (BIO-SAVE®), Pseudomonas viridiflava, Pseudozyma flocculosa strain PF-A 22 UL (SPORODEX L®), Puccinia canaliculata, Puccinia thlaspeos (WOOD WARRIOR®), Pythium paroecandrum, Pythium oligandrum (POLYGANDRON®, POLYVERSUM®), Pythium periplocum, Rhanella aquatilis, Rhanella spp., Rhizobia (DORMAL®, VAULT®), Rhizoctonia, Rhodococcus globerulus strain AQ719, Rhodosporidium diobovatum, Rhodosporidium toruloides, Rhodotorula glutinis, Rhodotorula graminis, Rhodotorula mucilagnosa, Rhodotorula rubra, Rhodotorula spp., Saccharomyces cerevisiae, Salinococcus roseus, Sclerotinia minor (SARRITOR®), Sclerotinia minor, Scytalidium spp., Scytalidium uredinicola, Serratia marcescens, Serratia plymuthica, Serratia spp., Sordaria fimicola, Spodoptera exigua nuclear polyhedrosis virus (SPOD-X®, SPEXIT®), Spodoptera littoralis nucleopolyhedrovirus (LITTOVIR®), Sporobolomyces roseus, Stenotrophomonas maltophilia, Streptomyces albaduncus, Streptomyces exfoliates, Streptomyces galbus, Streptomyces griseoplanus, Streptomyces griseoviridis (MYCOSTOP®), Streptomyces hygroscopicus, Streptomyces lydicus (ACTINOVATE®), Streptomyces lydicus WYEC-108 (ACTINOGROW®), Streptomyces violaceus, Tilletiopsis minor, Tilletiopsis spp., Trichoderma asperellum (T34 BIOCONTROL®), Trichoderma atroviride (PLANTMATE®), Trichoderma gamsii (TENET®), Trichoderma hamatum TH 382, Trichoderma harzianum rifai (MYCOSTAR®), Trichoderma harzianum T-22 (TRIANUM-P®, PLANTSHIELD HC®, ROOTSHIELD®, TRIANUM-G®), Trichoderma harzianum T-39 (TRICHODEX®), Trichoderma inhamatum, Trichoderma koningii, Trichoderma lignorum, Trichoderma longibrachiatum, Trichoderma polysporum (BINA BT®), Trichoderma spp. LC 52 (SENTINEL®), Trichoderma taxi, Trichoderma virens (formerly Gliocladium virens GL-21) (SOILGUARD®), Trichoderma virens, Trichoderma viride, Trichoderma viride strain ICC 080 (REMEDIER®), Trichosporon pullulans, Trichosporon spp., Trichothecium roseum, Trichothecium spp., Typhula phacorrhiza strain 94670, Typhula phacorrhiza strain 94671, Ulocladium atrum, Ulocladium oudemansii (BOTRY-ZEN®), Ustilago maydis, various bacteria and supplementary micronutrients (NATURAL II®), various fungi (MILLENNIUM MICROBES®), Vip3Aa20, Verticillium chlamydosporium, Virgibaclillus marismortui, Xanthomonas campestris pv. Poae (CAMPERICO®), Xenorhabdus bovienii, Xenorhabdus nematophilus; AGNIQUE® MMF, azadirachtin (PLASMA NEEM OIL®, AZAGUARD®, MEEMAZAL®, MOLT-X® e.g. AZATIN XL from Certis, US), Botanical IGR (NEEMAZAD®, NEEMIX®), BUGOIL®, canola oil (LILLY MILLER VEGOL®), Chenopodium ambrosioides near ambrosioides (REQUIEM®), Chrysanthemum extract (CRISANT®), essentials oils of Labiatae (BOTANIA®), extract of neem oil (TRILOGY®), extracts of clove rosemary peppermint and thyme oil (GARDEN INSECT KILLER®), garlic, Glycinebetaine (GREENSTIM®), kaolin (SCREEN®), lemongrass oil (GREENMATCH®), Melaleuca alternifolia extract (also called tea tree oil) (TIMOREX GOLD®), mixture of clove 81aponaria81 garlic oil and mint (SOIL SHOT®), mixture of clove rosemary and peppermint extract (EF 400®), mixture of rosemary sesame 81aponaria81 thyme and cinnamon extracts (EF 300®), neem oil, Nepeta cataria (Catnip oil), Nepeta 81aponari, nicotine, oregano oil (MOSSBUSTER®), Pedaliaceae oil (NEMATON®), pine oil (RETENOL®), pyrethrum, Quillaja 81aponaria (NEMAQ®), Reynoutria sachalinensis (REGALIA®, SAKALIA®), rotenone (ECOROTEN®), Rutaceae plant extract (SOLEO®), soybean oil (ORTHO ECOSENSE®), storage glucam of brown algae (LAMINARIN®), thyme oil; (E,Z)-7,9-Dodecadien-1-yl acetate, (E,Z,Z)-3,8,11 Tetradecatrienyl acetate, (Z,Z,E)-7,11,13-Hexadecatrienal, 2-M ethyl-1-butanol, BIOLURE®, blackheaded fireworm pheromone (3M SPRAYABLE BLACKHEADED FIREWORM PHEROMONE®), Calcium acetate, CHECK-MATE®, Codling Moth Pheromone (Paramount dispenser-(CM)/Isomate C-Plus®), Entostat powder (extract from palm tree) (EX OSEX CM®), Grape Berry Moth Pheromone (3M MEC-GBM SPRAYABLE PHEROMONE®), Lavandulyl senecioate, Leafroller pheromone (3M MEC-LR SPRAY ABLE PHEROMONE®), Muscamone (SNIP7 FLY BAIT®), Oriental Fruit M oth Pheromone (3M ORIENTAL FRUIT MOTH SPRAY ABLE PHEROMONE®), Peachtree Borer Pheromone (ISOMATE-PR), SCENTURION®, STARBAR PREMIUM FLY BAIT®), Tomato Pinworm Pheromone (3M SPRAY ABLE PHEROMONE®); Acerophagus papaya, Adalia bipunctata (ADALIA-SYSTEM®), Adalia bipunctata (ADALINE®), Adalia bipunctata (APHIDALIA®), Ageniaspis citricola, Ageniaspis fuscicollis, Amblyseius andersoni (ANDERLINE®, ANDERSONI-SYSTEM®), Amblyseius californicus (AMBLYLINE®, SPICAL®), Amblyseius cucumeris (THRIPEX®, BUGLINE CUCUMERIS®), Amblyseius fallacis (FALLACIS®), Amblyseius swirskii (BUGLINE SWIRSKII®, SWIRSKII-MITE®), Amblyseius womersleyi (WOMERMITE®), Amitus hesperidum, Anagrus atomus, Anagyrus fusciventris, Anagyrus kamali, Anagyrus loecki, Anagyrus pseudococci (CITRIPAR®), Anicetus benefices, Anisopteromalus calandrae, Anthocoris nemoralis (ANTHOCORIS-SYSTEM®), Aphelinus abdominalis (APHELINE®, APHILINE®), Aphelinus asychis, Aphidius colemani (APHIPAR®), Aphidius ervi (APHELINUS-SYSTEM®), Aphidius ervi (ERVIPAR®), Aphidius gifuensis, Aphidius matricariae (A PHIPAR-M®), Aphidoletes aphidimyza (APHIDEND®, APHIDOLINE®), Aphytis lingnanensis, Aphytis melinus, Aprostocetus hagenowii, Atheta coriaria (STAPHYLINE®), Bombus spp., Bombus terrestris (BEELINE®, TRIPOL®), Bombus terrestris (NATUPOL BEEHIVE®), Cephalonomia stephanoderis, Chilocorus nigritus, Chrysoperla carnea (CHRYSOLINE®, CHRYSOPA®), Chrysoperla rufilabris, Cirrospilus ingenuus, Cirrospilus quadristriatus, Citrostichus phyllocnistoides, Closterocerus chamaeleon, Closterocerus spp., Coccidoxenoides perminutus (PLANOPAR®), Coccophagus cowperi, Coccophagus lycimnia, Cotesia flavipes, Cotesia plutellae, Cryptolaemus montrouzieri (CRYPTOBUG®, CRYPTOLINE®), Cybocephalus nipponicus, Dacnusa sibirica (MINUSA®, DACDIGLINE®, MINEX®), Delphastus catalinae (DELPHASTUS®), Delphastus pusillus, Diachasmimorpha krausii, Diachasmimorpha longicaudata, Diaparsis jucunda, Diaphorencyrtus aligarhensis, Diglyphus isaea (DIMINEX®, MIGLYPHUS®, DIGLINE®), Diversinervus spp., Encarsia citrina, Encarsia formosa (ENCARSIA MAX®, ENCARLINE®, EN-STRIP®), Encarsia guadeloupae, Encarsia haitiensis, Episyrphus balteatus (SYRPHIDEND®), Eretmoceris siphonini, Eretmocerus californicus, Eretmocerus eremicus (ENERMIX®, ERCAL®, ERETLINE E®, BEMIMIX®), Eretmocerus hayati, Eretmocerus mundus (BEMIPAR®, ERETLINE M®), Eretmocerus siphonini, Exochomus quadripustulatus, Feltiella acarisuga (FELTILINE®), Feltiella acarisuga (SPIDEND®), Fopius arisanus, Fopius ceratitivorus, Formononetin (WIRLESS BEEHOME®), Franklinothrips vespiformis (VESPOP®), Galendromus occidentalis, Goniozus legneri, Habrobracon hebetor, Harmonia axyridis (HARMOBEETLE®), Heterorhabditis bacteriophora (NEMASHIELD HB®, NEMASEEK®, TERRANEM-NAM®, TERRANEM®, LARVANEM®, B-GREEN®, NEMATTACK®, NEMATOP®), Heterorhabditis megidis (NEMASYS H®, BIONEM H®, EXHIBITLINE HM®, LARVANEM-M®), Heterorhabditis spp. (LAWN PATROL®), Hippodamia convergens, Hypoaspis aculeifer (ACULEIFER-SYSTEM®, ENTOMITE-A®), Hypoaspis miles (HYPOLINE M®, ENTOMITE-M®), Lbalia leucospoides, Lecanoideus floccissimus, Lemophagus errabundus, Leptomastidea abnormis, Leptomastix dactylopii (LEPTOPAR®), Leptomastix epona, Lindorus lophanthae, Lipolexis oregmae, Lucilia caesar (NATUFLY®), Lysiphlebus testaceipes, Macrolophus caliginosus (MIRICAL-N®, MACROLINE C®, MIRICAI®), Mesoseiulus longipes, Metaphycus flavus, Metaphycus lounsburyi, Micromus angulatus (MILACEWING®), Microterys flavus, Muscidifurax raptorellus and Spalangia cameroni (BIOPAR®), Neodryinus typhlocybae, Neoseiulus californicus, Neoseiulus cucumeris (THRY PEX®), Neoseiulus fallacis, Nesideocoris tenuis (NESIDIOBUG®, NESIBUG®), Ophyra aenescens (BIOFLY®), Orius insidiosus (THRIPOR-I®, ORILINE I®), Orius laevigatus (THRIPOR-L®, ORILINE L®), Orius majusculus (ORILINE M®), Orius strigicollis (THRIPOR-S®), Pauesia juniperorum, Pediobius foveolatus, Phasmarhabditis hermaphrodita (NEMASLUG®), Phymastichus coffea, Phytoseiulus macropilus, Phytoseiulus persimilis (SPIDEX®, PHYTOLINE P®), Podisus maculiventris (PODISUS®), Pseudacteon curvatus, Pseudacteon obtusus, Pseudacteon tricuspis, Pseudaphycus maculipennis, Pseudleptomastix mexicana, Psyllaephagus pilosus, Psyttalia concolor (complex), Quadrastichus spp., Rhyzobius lophanthae, Rodolia cardinalis, Rumina decollate, Semielacher petiolatus, Sitobion avenae (ERVIBANK®), Steinernema carpocapsae (NEMATAC C®, MILLENIUM®, BIONEM C®, NEMATTACK®, NEMASTAR®, CAPSANEM®), Steinernema feltiae (NEMASHIELD®, NEMASYS F®, BIONEM F®, STEINERNEMA-SYSTEM®, NEMATTACK®, NEMAPLUS®, EXHIBITLINE SF®, SCIA-RID®, ENTONEM®), Steinernema kraussei (NEMASYS L®, BIONEM L®, EXHIBITLINE SRB®), Steinernema riobrave (BIOVECTOR®, BIOVEKTOR®), Steinernema scapterisci (NEMATACS®), Steinernema spp., Steinernematid spp. (GUARDIAN NEMATODES®), Stethorus punctillum (STETHORUS®), Tamarixia radiate, Tetrastichus setifer, Thripobius semiluteus, Torymus sinensis, Trichogramma brassicae (TRICHOLINE B®), Trichogramma brassicae (TRICHO-STRIP®), Trichogramma evanescens, Trichogramma minutum, Trichogramma ostriniae, Trichogramma platneri, Trichogramma pretiosum, Xanthopimpla stemmator; abscisic acid, AMINOMITE®, BIOGAIN®, BIOSEA®, CAS Number: 2643947-26-4, Chondrostereum purpureum (CHONTROL PASTE®), Colletotrichum gloeosporioides (COLLEGO®), Copper Octanoate (CUEVA®), Erwinia amylovora (Harpin) (ProAct®, Ni-HIBIT Gold CST®), fatty acids derived from a natural by-product of extra virgin olive oil (FLIPPER®), Ferri-phosphate (FERRAMOL®), Homo-brassonolide, Iron Phosphate (LILLY MILLER WORRY FREE FERRAMOL SLUG & SNAIL BAIT®), Microctonus hyperodae, Mycoleptodiscus terrestris (DES-X®), Nosema locustae (SEMASPORE ORGANIC GRASSHOPPER CONTROL®), potassium bicarbonate (MILSTOP®), potassium iodide+potassiumthiocyanate (ENZICUR®), potassium salts of fatty acids (SANOVA®), potassium silicate solution (SIL-MATRIX®), Spider venom, Bacillus mojavensis strain R3B (Accession No. NCAIM (P) B 001389) (WO 2013/034938) from Certis USA LLC, Bacillus pumilus, in particular strain BU F-33, having NRRL Accession No. 50185 (CARTISSA® from BASF, EPA Reg. No. 71840-19), Bacillus subtilis CX-9060 from Certis USA LLC, Bacillus sp., in particular strain D 747 (available as DOUBLE NICKEL® from Kumiai Chemical Industry Co., Ltd.), having Accession No. FERM BP-8234, U.S. Pat. No. 7,094,592, Bacillus subtilis strain BU 1814, (VELONDIS® PLUS, VELONDIS® FLEX and VELONDIS® EXTRA from BASF SE), Bacillus subtilis var. amyloliquefaciens strain FZB 24 having Accession No. DSM 10271 (available from Novozymes as TAEGRO® or TAEGRO® ECO (EPA Registration No. 70127-5)), Bacillus subtilis, in particular strain QST713/AQ713 (having NRRL Accession No. B-21661 and described in U.S. Pat. No. 6,060,051, available as SERENADE® OPTI or SERENADE® ASO from Bayer CropScience LP, US), Paenibacillus polymyxa, in particular strain AC-1 (e.g. TOPSEED® from Green Biotech Company Ltd.), Paenibacillus sp. strain having Accession No. NRRL B-50972 or Accession No. NRRL B-67129, WO 2016/154297, Pantoea agglomerans, in particular strain E 325 (Accession No. NRRL B-21856) (available as BLOOMTIME BIOLOGICAL™ FD BIOPESTICIDE from Northwest Agri Products), Pseudomonas proradix (e.g. PRORADIX® from Sourcon Padena); Aureobasidium pullulans, in particular blastospores of strain DSM 14940, blastospores of strain DSM 14941 or mixtures of blastospores of strains DSM 14940 and DSM 14941 (e.g., BOTECTOR® and BLOSSOM PROTECT® from bio-ferm, CH), Pseudozyma aphidis (as disclosed in WO2011/151819 by Yissum Research Development Company of the Hebrew University of Jerusalem), Saccharomyces cerevisiae, in particular strains CNCM No. 1-3936, CNCM No. 1-3937, CNCM No. 1-3938 or CNCM No. 1-3939 (WO 2010/086790) from Lesaffre et Compagnie, FR; Agrobacterium radiobacter strain K 84 (e.g. GALLTROL-A® from AgBioChem, CA), Bacillus amyloliquefaciens isolate B 246 (e.g. AVOGREEN™ from University of Pretoria), Bacillus amyloliquefaciens strain F727 (also known as strain M B1110) (NRRL Accession No. B-50768, WO 2014/028521) (STARGUS® from Marrone Bio Innovations), Bacillus amyloliquefaciens strain FZB 42, Accession No. DSM 23117 (available as RHIZOVITAL® from A BITEP, DE), Bacillus amyloliquefaciens, in particular strain D 747 (available as DOUBLE NICKEL™ from Kumiai Chemical Industry Co., Ltd., having accession number FERM BP-8234, U.S. Pat. No. 7,094,592), Bacillus licheniformis FMCH001 and Bacillus subtilis FMCH 002 (QUARTZO® (WG) and PRESENCE® (WP) from FMC Corporation), Bacillus licheniformis, in particular strain SB 3086, having Accession No. ATCC 55406, WO 2003/000051, Bacillus methylotrophicus strain BAC-9912 (from Chinese Academy of Sciences' Institute of Applied Ecology), Bacillus mycoides, isolate, having Accession No. B-30890 (available as BMJ TGAI® or WG and LIFEGARD™ from Certis USA LLC), Bacillus pumilus, in particular strain QST2808 (available as SONATA® from Bayer CropScience LP, US, having Accession No. NRRL B-30087 and described in U.S. Pat. No. 6,245,551), Bacillus subtilis CX-9060 from Certis USA LLC, Bacillus subtilis IAB/BS03 (AVIV™ from STK Bio-Ag Technologies, PORTENTO® from Idai Nature), Bacillus subtilis KTSB strain (FOLIACTIVE® from Donaghys), Bacillus subtilis strain BU 1814, (available as VELONDIS® PLUS, VELONDIS® FLEX and VELONDIS® EXTRA from BASF SE), Bacillus subtilis strain GB 03 (available as KODIAK® from Bayer AG, DE), Bacillus subtilis strain MBI 600 (available as SUBTILEX from BASF SE), having Accession Number NRRL B-50595, U.S. Pat. No. 5,061,495, Bacillus subtilis strain Y 1336, Bacillus subtilis var. amyloliquefaciens strain FZB 24 having Accession No. DSM 10271 (available from Novozymes as TAEGRO® or TAEGRO® ECO (EPA Registration No. 70127-5)), Paenibacillus epiphyticus (WO 2016/020371) from BASF SE, Paenibacillus polymyxa ssp. plantarum (WO 2016/020371) from BASF SE, Paenibacillus sp. strain having Accession No. NRRL B-50972 or Accession No. NRRL B-67129, WO 2016/154297, Pseudomonas chlororaphis strain A FS009, having Accession No. NRRL B-50897, WO 2017/019448 (e.g., HOWLER™ and ZIO® from AgBiome Innovations, US), Pseudomonas chlororaphis, in particular strain MA 342 (e.g. CEDOMON®, CERALL®, and CEDRESS® by Bioagri and Koppert), Pseudomonas fluorescens strain A 506 (e.g. BLIGHTBAN® A 506 by NuFarm), Pseudomonas proradix (e.g. PRORADIX® from Sourcon Padena), Streptomyces griseoviridis strain K 61 (also known as Streptomyces galbus strain K 61) (Accession No. DSM 7206) (MYCOSTOP® from Verdera, PREFENCE® from BioWorks, cf. Crop Protection 2006, 25, 468-475), Streptomyces lydicus strain WYEC108 (also known as Streptomyces lydicus strain WY CD 108US) (ACTINO-IRON® and ACTINOVATE® from Novozymes); Trichoderma atroviride strain T11 (IM 1352941/CECT 20498), Ampelomyces quisqualis strain AQ10, having Accession No. CNCM 1-807 (e.g., AQ10® by IntrachemBio Italia), Ampelomyces quisqualis, in particular strain AQ10 (e.g. AQ10® by IntrachemBio Italia), Aspergillus flavus strain NRRL 21882 (products known as AFLA-GUARD® from Syngenta/ChemChina), Aureobasidium pullulans, in particular blastospores of strain DSM 14941, Aureobasidium pullulans, in particular blastospores of strain DSM 14940, Aureobasidium pullulans, in particular mixtures of blastospores of strains DSM 14940 and DSM 14941 (e.g. Botector® by bio-ferm, CH), Chaetomium cupreum (Accession No. CABI 353812) (e.g. BIOKUPRUM™ by AgriLife), Chaetomium globosum (available as RIVADIOM® by Rivale), Cladosporium cladosporioides, strain H 39, having Accession No. CBS122244, US 2010/0291039 (by Stichting Dienst Landbouwkundig Onderzoek), Coniothyrium minitans, in particular strain CON/M/91-8 (Accession No. DSM 9660, e.g. Contans® from Bayer CropScience Biologics GmbH), Cryptococcus flavescens, strain 3C (NRRL Y-50378),, Dactylaria candida, Dilophosphora alopecuri (available as TWIST FUNGUS®), Fusarium oxysporum, strain Fo47 (available as FUSA CLEAN® by Natural Plant Protection), Gliocladium catenulatum (Synonym: Clonostachys rosea f. catenulate) strain J 1446 (e.g. PRESTOP® by Lallemand), Gliocladium roseum (also known as Clonostachys rosea f rosea) strain IK 726 (Jensen D F, et al. Development of a biocontrol agent for plant disease control with special emphasis on the near commercial fungal antagonist Clonostachys rosea strain ‘IK726’, Australasian Plant Pathol. 2007,36 (2): 95-101), Gliocladium roseum (also known as Clonostachys rosea f rosea), in particular strain 321U from Adjuvants Plus, strain ACM 941 as disclosed in Xue A.G. (Can J our Plant Sci 2003, 83 (3): 519-524), Metschnikowia fructicola, in particular strain NRRL Y-30752, Microsphaeropsis ochracea, Penicillium steckii (DSM 27859, WO 2015/067800) from BASF SE, mixtures of Trichoderma asperellum strain ICC 012 (also known as Trichoderma harzianum ICC012), having Accession No. CABI CC IMI 392716 and Trichoderma gamsii (formerly T. viride) strain ICC 080, having Accession No. IMI 392151 (e.g., BIO-TAM™ from Isagro USA, Inc. or BIODERMA® by Agrobiosol de Mexico, S.A. de C.V.), Penicillium vermiculatum, Phlebiopsis gigantea strain VRA 1992 (ROTSTOP® C from Danstar Ferment), Pseudozyma flocculosa, strain PF-A 22 UL (available as SPORODEX® L by Plant Products Co., CA), Saccharomyces cerevisiae strain LAS117 cell walls (CEREVISANE® from Lesaffre, ROMEO® from BASF SE), Saccharomyces cerevisiae strains CNCM No. 1-3936, CNCM No. 1-3937, CNCM No. 1-3938, CNCM No. 1-3939 (WO 2010/086790) from Lesaffre et Compagnie, FR, Saccharomyces cerevisiae, in particular strain LASO2 (from Agro-Levures et Dérivés), Simplicillium lanosoniveum, strain T34 (e.g. T34 Biocontrol by Biocontrol Technologies S.L., ES) or strain ICC 012 from Isagro, strain WRL-076 (NRRL Y-30842), U.S. Pat. No. 7,579,183, Talaromyces flavus, strain V 117b, Trichoderma asperelloides JM 41R (Accession No. NRRL B-50759) (TRICHO PLUS® from BASF SE), Trichoderma asperellum, in particular strain SKT-1, having Accession No. FERM P-16510 (e.g. ECO-HOPE® from Kumiai Chemical Industry), Trichoderma asperellum, in particular, strain kd (e.g. T-Gro from Andermatt Biocontrol), Trichoderma atroviride strain 77B (T77 from Andermatt Biocontrol), Trichoderma atroviride strain ATCC 20476 (IMI 206040), Trichoderma atroviride strain LC52 (e.g. Tenet by Agrimm Technologies Limited), Trichoderma atroviride strain LU 132 (e.g. Sentinel from Agrimm Technologies Limited), Trichoderma atroviride strain NMI no. V 08/002388, Trichoderma atroviride strain NMI no. V 08/002389, Trichoderma atroviride strain NMI no. V 08/002390, Trichoderma atroviride strain no. V 08/002387, Trichoderma atroviride strain SKT-1 (FERM P-16510), JP Patent Publication (Kokai) 11-253151 A, Trichoderma atroviride strain SKT-2 (FERM P-16511), JP Patent Publication (Kokai) 11-253151 A, Trichoderma atroviride strain SKT-3 (FERM P-17021), JP Patent Publication (Kokai) 11-253151 A, Trichoderma atroviride, in particular strain SC1 (Accession No. CBS 122089, WO 2009/116106 and U.S. Pat. No. 8,431,120 (from Bi-PA)), Trichoderma atroviride,strain CNCM 1-1237 (e.g. Esquive® WP from A grauxine, FR), Trichoderma fertile (e.g. product TrichoPlus from BASF), Trichoderma gamsii (formerly T. viride), Trichoderma gamsii (formerly T. viride) strain ICC 080 (IMICC 392151 CABI) (available as BIODERMA® by AGROBIOSOL DE MEXICO, S.A. DE C.V.),, Trichoderma gamsii strain ICC080 (IMI CC 392151 CABI, e.g. BioDerma by AGROBIOSOL DE MEXICO, S.A. DE C.V.),, Trichoderma harmatum, Trichoderma harmatum, having Accession No. ATCC 28012, Trichoderma harzianum, Trichoderma harzianum rifai T39 (e.g. TRICHODEX® from Makhteshim, US), Trichoderma harzianum strain Cepa SimbT5 (from Simbiose Agro), Trichoderma harzianum strain DB 103 (available as T-GRO® 7456 by Dagutat Biolab), Trichoderma harzianum strain ITEM 908 (e.g. Trianum-P from Koppert), Trichoderma harzianum strain T-22 (e.g. Trianum-P from Andermatt Biocontrol or Koppert), Trichoderma harzianum strain TH 35 (e.g. Root-Pro by Mycontrol), Trichoderma polysporum strain IMI 206039 (e.g. Binab TF WP by BINAB Bio-Innovation AB, Sweden), Trichoderma stromaticum having Accession No. Ts3550 (e.g. Tricovab by CEPLAC, Brazil), Trichoderma virens (also known as Gliocladium virens) in particular strain GL-21 (e.g. SoilGard by Certis, US), Trichoderma virens strain G-41, formerly known as Gliocladium virens (Accession No. ATCC 20906) (e.g., ROOTSHIELD® PLUS WP and TURFSHIELD® PLUS WP from BioWorks, US), Trichoderma viride in particular strain B35 (Pietr et al., 1993, Zesz. Nauk. A R w Szczecinie 161:125-137), Trichoderma viride strain TV 1 (e.g. Trianum-P by Koppert), Ulocladium oudemansii strain U3, having Accession No. NM 99/06216 (e.g., BOTRY-ZEN® by Botry-Zen Ltd, New Zealand and BOTRY STOP® from BioWorks, Inc.), Verticillium albo-atrum (formerly V. dahliae) strain WCS850 having Accession No. WCS850, deposited at the Central Bureau for Fungi Cultures (e.g., DUTCH TRIG® by Tree Care Innovations), Verticillium chlamydosporium; a mixture of Azotobacter vinelandii and Clostridium pasteurianum (available as INVIGORATE® from Agrinos), a mixture of Bacillus licheniformis FMCH001 and Bacillus subtilis FMCH002 (available as QUARTZO® (WG), PRESENCE® (WP) from FMC Corporation), Azorhizobium caulinodans, in particular strain ZB-SK-5, Azospirillum brasilense (e.g., VIGOR® from KALO, Inc.), Azospirillum lipoferum (e.g., VERTEX-IF™ from TerraMax, Inc.), Azotobacter chroococcum, in particular strain H23, Azotobacter vinelandii, in particular strain ATCC 12837, Bacillus amyloliquefaciens BS27 (Accession No. NRRL B-5015), Bacillus amyloliquefaciens in particular strain FZB 42 (e.g. RHIZOVITAL® from ABiTEP, DE), Bacillus amyloliquefaciens in particular strain IN 937a, Bacillus amyloliquefaciens pm414 (LOLI-PEPTA® from Biofilm Crop Protection), Bacillus amyloliquefaciens SB 3281 (ATCC #PTA-7542, WO 2017/205258), Bacillus amyloliquefaciens TJ 1000 (available as QUIK ROOTS® from Novozymes), Bacillus cereus family member EE128 (NRRL No. B-50917), Bacillus cereus family member EE 349 (NRRL No. B-50928), Bacillus cereus in particular strain BP01 (ATCC 55675, e.g. MEPICHLOR® from Arysta Lifescience, US), Bacillus mycoides BT155 (NRRL No. B-50921), Bacillus mycoides BT46-3 (NRRL No. B-50922), Bacillus mycoides EE118 (NRRL No. B-50918), Bacillus mycoides EE141 (NRRL No. B-50916), Bacillus pumilus in particular strain QST2808 (Accession No. NRRL No. B-30087), Bacillus siamensis in particular strain K CTC 13613T, Bacillus subtilis in particular strain AQ 30002 (Accession No. NRRL No. B-50421 and described in U.S. patent application Ser. No. 13/330,576), Bacillus subtilis in particular strain AQ30004 (NRRL No. B-50455 and described in U.S. patent application Ser. No. 13/330,576), Bacillus subtilis in particular strain MBI 600 (e.g. SUBTILEX® from BASF SE), Bacillus subtilis rm303 (RHIZOMAX® from Biofilm Crop Protection), Bacillus subtilis strain BU 1814 (available as TEQUALIS® from BASF SE), Bacillus tequilensis in particular strain NII-0943, Bacillus thuringiensis BT013A (NRRL No. B-50924) also known as Bacillus thuringiensis 4Q7, Bradyrhizobium japonicum (e.g. OPTIMIZE® from Novozymes), Delftia acidovorans in particular strain RAY 209 (e.g. BIOBOOST® from Brett Young Seeds), Lactobacillus sp. (e.g. LACTOPLANT® from LactoPA FI), Mesorhizobium cicer (e.g., NODULATOR from BASF SE), Paenibacillus polymyxa in particular strain A C-1 (e.g. TOPSEED® from Green Biotech Company Ltd.), Pseudomonas aeruginosa in particular strain PN1, Pseudomonas proradix (e.g. PRORADIX® from Sourcon Padena), Rhizobium leguminosarium biovar viciae (e.g., NODULATOR from BASF SE), Rhizobium leguminosarum in particular bv. viceae strain Z25 (Accession No. CECT 4585), Serratia marcescens in particular strain SRM (Accession No. MTCC 8708),, Sinorhizobium meliloti strain NRG-185-1 (NITRAGIN® GOLD from Bayer CropScience), Thiobacillus sp. (e.g. CROPAID® from Cropaid Ltd UK); Myrothecium verrucaria strain AARC-0255 (e.g. DiTera™ from Valent Biosciences), Penicillium bilaii strain ATCC 22348 (e.g. JumpStart® from Acceleron BioAg), Penicillium bilaii strain ATCC ATCC20851, Purpureocillium lilacinum (previously known as Paecilomyces lilacinus) strain 251 (A GAL 89/030550, e.g. BioAct from Bayer CropScience Biologics GmbH), Pythium oligandrum strain DV 74, Pythium oligandrum strain M1 (ATCC 38472 e.g. Polyversum from Bioprepraty, CZ), Rhizopogon amylopogon (Myco-Sol from Agri-Enterprise, LLC, formerly Helena Chemical Company), Rhizopogon fulvigleba (e.g. Myco-Sol from Agri-Enterprise, LLC, formerly Helena Chemical Company), Talaromyces flavus strain V117b, Trichoderma asperellum strain (Eco-T from Plant Health Products, ZA), Trichoderma asperellum strain kd (e.g. T-Gro from Andermatt Biocontrol), Trichoderma atroviride in particular strain no. V 08/002387, Trichoderma atroviride strain CNCM 1-1237 (e.g. Esquive® WP from A grauxine, FR), Trichoderma atroviride strain LC52 (also known as Trichoderma atroviride strain LU 132, e.g. Sentinel from Agrimm Technologies Limited), Trichoderma atroviride strain no. NMI No. V 08/002388, Trichoderma atroviride strain no. NMI No. V 08/002389, Trichoderma atroviride strain no. NMI No. V 08/002390, Trichoderma atroviride strain SC1 (described in WO 2009/116106), Trichoderma harzianum strain 1295-22, Trichoderma harzianum strain ITEM 908, Trichoderma harzianum strain T-22 (e.g. Trianum-P from Andermatt Biocontrol or Koppert), Trichoderma harzianum strain TSTh20,, Trichoderma virens strain GI-3, Trichoderma virens strain GL-21 (e.g. SoilGard® from Certis, USA), Trichoderma viride strain B 35 (Pietr et al., 1993, Zesz. Nauk. A R w Szczecinie 161:125-137), Verticillium albo-atrum (formerly V. dahliae) strain WCS850 (CBS 276.92, e.g. Dutch Trig from Tree Care Innovations); Agrobacterium radiobacter strain K 84 (Galltrol from AgBiochem Inc.), Bacillus amyloliquefaciens in particular strain PTS-4838 (e.g. AVEO from Valent Biosciences, US),, Bacillus mycoides, isolate J. (e.g. BmJ from Certis USA LLC),, Bacillus sphaericus in particular Serotype H5a5b strain 2362 (strain ABTS-1743) (e.g. VECTOLEX® from Valent BioSciences, US), Bacillus thuringiensis israelensis strain BMP 144 (e.g. AQUABAC® by Becker Microbial Products IL), Bacillus thuringiensis subsp. aizawai strain GC-91, Bacillus thuringiensis subsp. aizawai, in particular serotype H-7 (e.g. FLORBAC® WG from Valent BioSciences, US), Bacillus thuringiensis subsp. aizawai, in particular strain ABTS-1857 (SD-1372, e.g. XENTARI® from Valent BioSciences), Bacillus thuringiensis subsp. israelensis (serotype H-14) strain A M 65-52 (Accession No. ATCC 1276) (e.g. VECTOBAC® by Valent BioSciences, US), Bacillus thuringiensis subsp. kurstaki strain ABTS 351, Bacillus thuringiensis subsp. kurstaki strain BMP 123 (from Becker Microbial Products, IL, BARITONE from Bayer CropScience), Bacillus thuringiensis subsp. kurstaki strain EG 2348 (LEPINOX from Certis, US), Bacillus thuringiensis subsp. kurstaki strain EG 7841 (CRY MAX from Certis, US), Bacillus thuringiensis subsp. kurstaki strain HD-1 (e.g. DIPEL® ES from Valent BioSciences, US), Bacillus thuringiensis subsp. kurstaki strain PB 54, Bacillus thuringiensis subsp. kurstaki strain SA 11 (JAVELIN from Certis, US), Bacillus thuringiensis subsp. kurstaki strain SA 12 (THURICIDE from Certis, US), Bacillus thuringiensis subsp. tenebrionis strain NB 176 (SD-5428, e.g. NOVODOR® FC from BioFa DE), Bacillus thuringiensis var. Colmeri (e.g. TIANBAOBTC by Changzhou Jianghai Chemical Factory), Bacillus thuringiensis var. japonensis strain Buibui, Bacillus thuringiensis var. kurstaki strain EVB-113-19 (e.g., BIOPROTEC® from AEF Global), Brevibacillus laterosporus, Burkholderia spp. in particular Burkholderia rinojensis strain A 396 (also known as Burkholderia rinojensis strain MBI 305) (Accession No. NRRL B-50319, WO 2011/106491 and WO 2013/032693, e.g. MBI206 TGAI and ZELTO® from Marrone Bio Innovations),, Chromobacterium subtsugae in particular strain PRAA 4-1T (e.g. MBI-203, e.g. GRANDEVO® from Marrone Bio Innovations), Lecanicillium muscarium Ve6 (MYCOTAL from Koppert), Paenibacillus popilliae (formerly Bacillus popilliae, e.g. MILKY SPORE POWDER™ or MILKY SPORE GRANULAR™ from St. Gabriel Laboratories), Serratia entomophila (e.g. INVADE® by Wrightson Seeds), Serratia marcescens in particular strain SRM (Accession No. MTCC 8708), Trichoderma asperellum (TRICHODERMAX from Novozymes), Wolbachia pipientis ZAP strain (e.g., ZAP MALES® from MosquitoMate); Beauveria bassiana strain ATCC 74040 (e.g. NATURALIS® from Intrachem Bio Italia), Beauveria bassiana strain ATP02 (Accession No. DSM 24665), Apopka 97 (PREFERAL from SePRO), Beauveria bassiana strain GHA (Accession No. ATCC74250, e.g. BOTANIGUARD® ES and MY CONTROL-O® from Laverlam International Corporation), Metarhizium anisopliae 3213-1 (deposited under NRRL accession number 67074 disclosed in WO 2017/066094, Pioneer Hi-Bred International), Metarhizium robertsil 15013-1 (deposited under NRRL accession number 67073), Metarhizium robertsii 23013-3 (deposited under NRRL accession number 67075), Paecilomyces lilacinus strain 251 (MELOCON from Certis, US); Cydia pomonella (codling moth) granulosis virus (GV), Helicoverpa armigera (cotton bollworm) nuclear polyhedrosis virus (NPV), of Adoxophyes orana (summer fruit tortrix) granulosis virus (GV), Spodoptera exigua (beet armyworm) mNPV, Spodoptera frugiperda (fall armyworm) mNPV; Burkholderia spp. in particular Burkholderia cepacia (formerly known as Pseudomonas cepacia), Gigaspora spp., Glomus spp., Laccaria spp., LactoBacillus buchneri, Paraglomus spp., Pisolithus tinctorus, Pseudomonas spp., Rhizobium spp. in particular Rhizobium trifolii, Rhizopogon spp., Scleroderma spp., Streptomyces spp., Suillus spp., Agrobacterium spp., Azorhizobium caulinodans, Azospirillum spp., Azotobacter spp., Bradyrhizobium spp., Gigaspora monosporum; Allium sativum (NEM GUARD from Eco-Spray, BRALIC from ADAMA), Armour-Zen, Artemisia absinthium, Biokeeper WP, Brassicaceae extract in particular oilseed rape powder or mustard powder, Cassia nigricans, Celastrus angulatus, Chenopodium anthelminticum, Chenopodium quinoa saponin extract from quinoa seeds (e.g. Heads Up® (Saponins of Quinoa) from Heads Up plant Protectants, CA), Chitin, Dryopteris filix-mas, Equisetum arvense, Fortune A za, Fungastop, Melaleuca alternifolia extract (TIMOREX GOLD from STK), naturally occurring Blad polypeptide extracted from Lupin seeds (FRACTURE® from FMC), naturally occurring Blad polypeptide extracted from Lupin seeds (PROBLAD® from Certis EU), Pyrethrins, Quassia amara, Quercus, Quillaja extract (QL AGRI 35 from BASF), REGALIA MAXX from Marrone Bio), Requiem™ Insecticide, Reynoutria sachalinensis extract (REGALLIA, ryania/ryanodine, Symphytum officinale, Tanacetum vulgare, Thymol, Thymol mixed with Geraniol (CEDROZ from Eden Research), Thymol mixed with Geraniol and Eugenol (MEVALONE from Eden Research), Triact 70, TriCon, Tropaeulum majus, Urtica dioica, Veratrin, Viscum album; mercuric oxide, octhilinone, thiophanate-methyl; MGK 264, 2-(2-butoxyethoxy)-ethyl piperonylate, 2-isovalerylindan-1,3-dione, 4-(quinoxalin-2-ylamino)benzenesulfonamide, 5-(1,3-benzodioxol-5-yl)-3-hexylcyclohex-2-enone, acibenzolar, acibenzolar-S-methyl, alpha-bromadiolone, alpha-chlorohydrin, aluminium phosphide, anthraquinone, antu, arsenous oxide, barium carbonate, benoxacor, bisthiosemi, brodifacoum, bromadiolone, bromethalin, calcium cyanide, chloralose, chlorophacinone, cholecalciferol, cloquintocet (including cloquintocet-mexyl), copper naphthenate, copper oxychloride, coumachlor, coumafuryl, coumatetralyl, crimidine, cyprosulfamide, diazinon, dichlormid, dicyclopentadiene, difenacoum, difethialone, diphacinone, ergocalciferol, farnesol, farnesol with nerolidol, fenchlorazole (including fenchlorazole-ethyl), fenclorim, flocoumafen, fluoroacetamide, flupropadine, flupropadine hydrochloride, fluxofenim, furilazole, gamma-HCH, guazatine, guazatine acetates, HCH, hydrogen cyanide, imanin, iodomethane, isoxadifen (including isoxadifen-ethyl), lindane, magnesium phosphide, M B-599, mefenpyr (including mefenpyr-diethyl), metcamifen, methiocarb, methyl bromide, nerolidol, norbormide, petroleum oils, phosacetim, phosphine, phosphorus, pindone, piperonyl butoxide, piprotal, potassium arsenite, probenazole, propyl isomer, pyridin-4-amine, pyrinuron, Reynoutria sachalinensis extract, ribavirin, S421, scilliroside, sesamex, sesasmolin, sodium arsenite, sodium cyanide, sodium fluoro-acetate, strychnine, sulfoxide, thallium sulfate, thiram, trimethacarb, warfarin, zinc naphthenate, zinc phosphide, or ziram.

In some embodiments, the invention encompasses a corn plant or seed comprising elite event MZIR260 treated with an insecticide, where the insecticide comprises one or more of imidacloprid, sulfoxaflor, vitavax, permethrin, carboxin, metalaxyl, thiamethoxam, cyantraniliprole, chlorantraniliprole, clothianidin, tebuprimphos, cyfluthrin, terbufos, feluthrin, ethoprop, phorate, bifenthrin, bronflanilide, phorate, esfenvalerate, beta-cyfluthrin, alpha-cypermethrin, zeta cypermethrin, thiodicarb, gamma cyhalothrin, lambda-cyhalothrhin, flubendiamide, carbaryl, chlorantraniliprole, methoxyfenozide, methomyl, spinetoram, indoxacarb, and spinosad. In a further embodiment, the invention encompasses a corn plant or seed comprising elite event MZIR260 treated with an insecticide, where the insecticide comprises one or more of thiamethoxam, sulfoxaflor, lambda-cyhalothrin, cyantraniliprole, chlorantraniliprole, clothianidin, imidacloprid, thiodicarb and tefluthrin.

In one embodiment, the invention comprises a method for protecting a corn plant comprising elite event MZIR260 against a fungus, said method comprising (a) providing a corn seed or plant comprising elite event MZIR260; and (b) treating the seed or plant with a fungicide. In some embodiments, the invention encompasses a corn plant or seed comprising elite event MZIR260 treated with one or more fungicides. In some embodiments, the fungicide is or comprises one or more of the following: petroleum oils, 1,1-bis(4-chloro-phenyl)-2-ethoxyethanol, 2,4-dichlorophenyl benzenesulfonate, 2-fluoro-N-methyl-N-1-naphthylacetamide, 4-chlorophenyl phenyl sulfone, acetoprole, aldoxycarb, amidithion, amidothioate, amiton, amiton hydrogen oxalate, amitraz, aramite, arsenous oxide, azobenzene, azothoate, benomyl, benoxa-fos, benzyl benzoate, bixafen, brofenvalerate, bromo-cyclen, bromophos, bromopropylate, buprofezin, butocarboxim, butoxycarboxim, butylpyridaben, calcium polysulfide, camphechlor, carbanolate, carbophenothion, cymiazole, chino-methionat, chlorbenside, chlordimeform, chlordimeform hydrochloride, chlorfenethol, chlorfenson, chlorfensulfide, chlorobenzilate, chloromebuform, chloromethiuron, chloropropylate, chlorthiophos, cinerin I, cinerin II, cinerins, closantel, coumaphos, crotamiton, crotoxyphos, cufraneb, cyanthoate, DCPM, DDT, demephion, demephion-O, demephion-S, demeton-methyl, demeton-O, demeton-O-methyl, demeton-S, demeton-S-methyl, demeton-S-methylsulfon, dichlofluanid, dichlorvos, dicliphos, dienochlor, dimefox, dinex, dinex-diclexine, dinocap-4, dinocap-6, dinocton, dino-penton, dinosulfon, dinoterbon, dioxathion, diphenyl sulfone, disulfiram, DNOC, dofenapyn, doramectin, endothion, eprinomectin, ethoate-methyl, etrimfos, fenazaflor, fenbutatin oxide, fenothiocarb, fenpyrad, fen-pyroximate, fenpyrazamine, fenson, fentrifanil, flubenzimine, flucycloxuron, fluenetil, fluorbenside, FMC 1137, formetanate, formetanate hydrochloride, formparanate, gamma-HCH, glyodin, halfenprox, hexadecyl cyclopropanecarboxylate, isocarbophos, jasmolin I, jasmolin II, jodfenphos, lindane, malonoben, mecarbam, mephosfolan, mesulfen, methacrifos, methyl bromide, metolcarb, mexacarbate, milbemycin oxime, mipafox, monocrotophos, morphothion, moxidectin, naled, 4-chloro-2-(2-chloro-2-methyl-propyl)-5-[(6-iodo-3-pyridyl) methoxy]pyridazin-3-one, nifluridide, nikkomycins, nitrilacarb, nitrilacarb 1:1 zinc chloride complex, omethoate, oxydeprofos, oxydisulfoton, pp′-DDT, parathion, permethrin, phenkapton, phosalone, phosfolan, phosphamidon, polychloroterpenes, polynactins, proclonol, promacyl, propoxur, prothidathion, prothoate, pyrethrin I, pyrethrin II, pyrethrins, pyridaphenthion, pyrimitate, quinalphos, quintiofos, R-1492, phosglycin, rotenone, schradan, sebufos, selamectin, sophamide, SSI-121, sulfiram, sulfluramid, sulfotep, sulfur, diflovidazin, tau-fluvalinate, TEPP, terbam, tetradifon, tetrasul, thiafenox, thiocarboxime, thiofanox, thiometon, thioquinox, thuringiensin, triamiphos, triarathene, triazophos, triazuron, trifenofos, trinactin, vamidothion, vaniliprole, bethoxazin, copper dioctanoate, copper sulfate, cybutryne, dichlone, dichlorophen, endothal, fentin, hydrated lime, nabam, quinoclamine, quinonamid, simazine, triphenyltin acetate, triphenyltin hydroxide, crufomate, piperazine, thiophanate, chloralose, fenthion, pyridin-4-amine, strychnine, 1-hydroxy-1H-pyridine-2-thione, 4-(quinoxalin-2-ylamino)benzenesulfonamide, 8-hydroxyquinoline sulfate, bronopol, copper hydroxide, cresol, dipyrithione, dodicin, fenaminosulf, formaldehyde, hydrargaphen, kasugamycin, kasugamycin hydrochloride hydrate, nickel bis(dimethyldithiocarbamate), nitrapyrin, octhilinone, oxolinic acid, oxytetracycline, potassium hydroxyquinoline sulfate, probenazole, streptomycin, streptomycin sesquisulfate, tecloftalam, thiomersal, Adoxophyes orana GV, Agrobacterium radiobacter, Amblyseius spp., Anagrapha falcifera NPV, Anagrus atomus, Aphelinus abdominalis, Aphidius colemani, Aphidoletes aphidimyza, Autographa californica NPV, Bacillus sphaericus Neide, Beauveria brongniartii, Chrysoperla carnea, Cryptolaemus montrouzieri, Cydia pomonella GV, Dacnusa sibirica, Diglyphus isaea, Encarsia formosa, Eretmocerus eremicus, Heterorhabditis bacteriophora and H. megidis, Hippodamia convergens, Leptomastix dactylopii, Macrolophus caliginosus, Mamestra brassicae NPV, Metaphycus helvolus, Metarhizium anisopliae var. acridum, Metarhizium anisopliae var. anisopliae, Neodiprion sertifer NPV and N. lecontei NPV, Orius spp., Paecilomyces fumosoroseus, Phytoseiulus persimilis, Steinernema bibionis, Steinernema carpocapsae, Steinernema feltiae, Steinernema glaseri, Steinernema riobrave, Steinernema riobravis, Steinernema scapterisci, Steinernema spp., Trichogramma spp., Typhlodromus occidentalis, Verticillium lecanii, apholate, bisazir, busulfan, dimatif, hemel, hempa, metepa, methiotepa, methyl apholate, morzid, penfluron, tepa, thiohempa, thiotepa, tretamine, uredepa, (E)-dec-5-en-1-yl acetate with (E)-dec-5-en-1-ol, (E)-tridec-4-en-1-yl acetate, (E)-6-methylhept-2-en-4-ol, (E,Z)-tetradeca-4,10-dien-1-yl acetate, (Z)-dodec-7-en-1-yl acetate, (Z)-hexadec-11-enal, (Z)-hexadec-11-en-1-yl acetate, (Z)-hexadec-13-en-11-yn-1-yl acetate, (Z)-icos-13-en-10-one, (Z)-tetradec-7-en-1-al, (Z)-tetradec-9-en-1-ol, (Z)-tetradec-9-en-1-yl acetate, (7E,9Z)-dodeca-7,9-dien-1-yl acetate, (9Z,11E)-tetradeca-9,11-dien-1-yl acetate, (9Z,12E)-tetradeca-9,12-dien-1-yl acetate, 14-methyloctadec-1-ene, 4-methylnonan-5-ol with 4-methylnonan-5-one, alpha-multistriatin, brevicomin, codlelure, codlemone, cuelure, disparlure, dodec-8-en-1-yl acetate, dodec-9-en-1-yl acetate, dodeca-8,10-dien-1-yl acetate, dominicalure, ethyl 4-methyloctanoate, eugenol, frontalin, grandlure, grandlure I, grandlure II, grandlure III, grandlure IV, hexalure, ipsdienol, ipsenol, japonilure, lineatin, litlure, looplure, medlure, megatomoic acid, methyl eugenol, muscalure, octadeca-2,13-dien-1-yl acetate, octadeca-3,13-dien-1-yl acetate, orfralure, oryctalure, ostramone, siglure, sordidin, sulcatol, tetradec-11-en-1-yl acetate, trimedlure, trimedlure A, trimedlure B 1, trimedlure B 2, trimedlure C, trunc-call, 2-(octylthio)-ethanol, butopyronoxyl, butoxy (polypropylene glycol), dibutyl adipate, dibutyl phthalate, dibutyl succinate, diethyltoluamide, dimethyl carbate, dimethyl phthalate, ethyl hexanediol, hexamide, methoquin-butyl, methylneodecanamide, oxamate, picaridin, 1-dichloro-1-nitroethane, 1,1-dichloro-2,2-bis(4-ethylphenyl)-ethane, 1,2-dichloropropane with 1,3-dichloropropene, 1-bromo-2-chloroethane, 2,2,2-trichloro-1-(3,4-dichloro-phenyl)ethyl acetate, 2,2-dichlorovinyl 2-ethylsulfinylethyl methyl phosphate, 2-(1,3-dithiolan-2-yl)phenyl dimethylcarbamate, 2-(2-butoxyethoxy)ethyl thiocyanate, 2-(4,5-dimethyl-1,3-dioxolan-2-yl)phenyl methylcarbamate, 2-(4-chloro-3,5-xylyloxy) ethanol, 2-chlorovinyl diethyl phosphate, 2-imidazolidone, 2-isovalerylindan-1,3-dione, 2-methyl (prop-2-ynyl)aminophenyl methylcarbamate, 2-thiocyanatoethyl laurate, 3-bromo-1-chloroprop-1-ene, 3-methyl-1-phenylpyrazol-5-yl dimethyl-carbamate, 4-methyl (prop-2-ynyl)amino-3,5-xylyl methylcarbamate, 5,5-dimethyl-3-oxocyclohex-1-enyl dimethylcarbamate, acethion, acrylonitrile, aldrin, allosamidin, allyxycarb, alpha-ecdysone, aluminium phosphide, aminocarb, anabasine, athidathion, azamethiphos, Bacillus thuringiensis delta endotoxins, barium hexafluorosilicate, barium polysulfide, barthrin, Bayer 22/190, Bayer 22408, beta-cyfluthrin, beta-cypermethrin, bioethanomethrin, biopermethrin, bis(2-chloroethyl) ether, borax, bromfenvinfos, bromo-D DT, bufencarb, butacarb, butathiofos, butonate, calcium arsenate, calcium cyanide, carbon disulfide, carbon tetrachloride, cartap hydrochloride, cevadine, chlorbicyclen, chlordane, chlordecone, chloroform, chloropicrin, chlorphoxim, chlorprazophos, cis-resmethrin, cismethrin, clocythrin, copper acetoarsenite, copper arsenate, copper oleate, coumithoate, cryolite, CS 708, cyanofenphos, cyanophos, cyclethrin, cythioate, d-tetramethrin, DAEP, dazomet, decarbofuran, diamidafos, dicapthon, dichlofenthion, dicresyl, dicyclanil, dieldrin, diethyl 5-methylpyrazol-3-yl phosphate, dilor, dimefluthrin, dimetan, dimethrin, dimethylvinphos, dimetilan, dinoprop, dinosam, dinoseb, diofenolan, dioxabenzofos, dithicrofos, DSP, ecdysterone, EI 1642, EMPC, EPBP, etaphos, ethiofencarb, ethyl formate, ethylene dibromide, ethylene dichloride, ethylene oxide, EXD, fenchlorphos, fenethacarb, fenitrothion, fenoxacrim, fenpirithrin, fensulfothion, fenthion-ethyl, flucofuron, fosmethilan, fospirate, fosthietan, furathiocarb, furethrin, guazatine, guazatine acetates, sodium tetrathiocarbonate, halfenprox, HCH, HEOD, heptachlor, heterophos, HHDN, hydrogen cyanide, hyquincarb, IPSP, isazofos, isobenzan, isodrin, isofenphos, isolane, isoprothiolane, isoxathion, juvenile hormone I, juvenile hormone II, juvenile hormone III, kelevan, kinoprene, lead arsenate, leptophos, lirimfos, lythidathion, m-cumenyl methylcarbamate, magnesium phosphide, mazidox, mecarphon, menazon, mercurous chloride, mesulfenfos, metam, metam-potassium, metam-sodium, methanesulfonyl fluoride, methocrotophos, methoprene, methothrin, methoxychlor, methyl isothiocyanate, methylchloroform, methylene chloride, metoxadiazone, mirex, naftalofos, naphthalene, NC-170, nicotine, nicotine sulfate, nithiazine, nornicotine, O-5-dichloro-4-iodophenyl O-ethyl ethylphosphonothioate, O,O-diethyl O-4-methyl-2-oxo-2H-chromen-7-yl phosphorothioate, O,O-diethyl O-6-methyl-2-propylpyrimidin-4-yl phosphorothioate, O,O,O′,O′-tetrapropyl dithiopyrophosphate, oleic acid, para-dichlorobenzene, parathion-methyl, pentachlorophenol, pentachlorophenyl laurate, PH 60-38, phenkapton, phosnichlor, phosphine, phoxim-methyl, pirimetaphos, polychlorodicyclopentadiene isomers, potassium arsenite, potassium thiocyanate, precocene I, precocene II, precocene III, primidophos, profluthrin, promecarb, prothiofos, pyrazophos, pyresmethrin, quassia, quinalphos-methyl, quinothion, rafoxanide, resmethrin, rotenone, kadethrin, ryania, ryanodine, sabadilla, schradan, sebufos, SI-0009, thiapronil, sodium arsenite, sodium cyanide, sodium fluoride, sodium hexafluorosilicate, sodium pentachlorophenoxide, sodium selenate, sodium thiocyanate, sulcofuron, sulcofuron-sodium, sulfuryl fluoride, sulprofos, tar oils, tazimcarb, TDE, tebupirimfos, temephos, terallethrin, tetrachloroethane, thicrofos, thiocyclam, thiocyclam hydrogen oxalate, thionazin, thiosultap, thiosultap-sodium, tralomethrin, transpermethrin, triazamate, trichlormetaphos-3, trichloronat, trimethacarb, tolprocarb, triclopyricarb, triprene, veratridine, veratrine, XMC, zetamethrin, zinc phosphide, zolaprofos, meperfluthrin, tetramethylfluthrin, bis (tributyltin) oxide, bromoacetamide, ferric phosphate, niclosamide-olamine, tributyltin oxide, pyrimorph, trifenmorph, 1,2-dibromo-3-chloropropane, 1,3-dichloropropene, 3,4-dichlorotetrahydrothio-phene 1,1-dioxide, 3-(4-chlorophenyl)-5-methylrhodanine, 5-methyl-6-thioxo-1,3,5-thiadiazinan-3-ylacetic acid, 6-isopentenylaminopurine, anisiflupurin, benclothiaz, cytokinins, DCIP, furfural, isamidofos, kinetin, Myrothecium verrucaria composition, tetrachlorothiophene, xylenols, zeatin, potassium ethylxanthate, acibenzolar, acibenzolar-S-methyl, Reynoutria sachalinensis extract, alpha-chlorohydrin, antu, barium carbonate, bisthiosemi, brodifacoum, bromadiolone, bromethalin, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, norbormide, phosacetim, phosphorus, pindone, pyrinuron, scilliroside, sodium fluoroacetate, thallium sulfate, warfarin,-2-(2-butoxyethoxy)ethyl piperonylate, 5-(1,3-benzodioxol-5-yl)-3-hexylcyclohex-2-enone, farnesol with nerolidol, verbutin, MGK 264, piperonyl butoxide, piprotal, propyl isomer, S421, sesamex, sesasmolin, sulfoxide, anthraquinone, copper naphthenate, copper oxychloride, dicyclopentadiene, thiram, zinc naphthenate, ziram, imanin, ribavirin, chloroinconazide, mercuric oxide, thiophanate-methyl, azaconazole, bitertanol, bromuconazole, cyproconazole, difenoconazole, diniconazole, epoxiconazole, fenbuconazole, fluquinconazole, flusilazole, flutriafol, furametpyr, hexaconazole, imazalil-, imiben-conazole, ipconazole, metconazole, myclobutanil, paclobutrazole, pefurazoate, penconazole, prothioconazole, pyrifenox, prochloraz, propiconazole, pyrisoxazole,-simeconazole, tebucon-azole, tetraconazole, triadimefon, triadimenol, triflumizole, triticonazole, ancymidol, fenarimol, nuarimol, bupirimate, dimethirimol, ethirimol, dodemorph, fenpropidin, fenpropimorph, spiroxamine, tridemorph, cyprodinil, mepanipyrim, pyrimethanil, fenpiclonil, fludioxonil, benalaxyl, furalaxyl, metalaxyl, R-metalaxyl, ofurace, oxadixyl, carbendazim, debacarb, fuberidazole, thiabendazole, chlozolinate, dichlozoline, myclozoline-, procymidone, vinclozoline, boscalid, carboxin, fenfuram, flutolanil, mepronil, oxycarboxin, penthiopyrad, thifluzamide, dodine, iminoctadine, azoxystrobin, dimoxystrobin, enestroburin, fenaminstrobin, flufenoxystrobin, fluoxastrobin, kresoxim-methyl, metominostrobin, trifloxystrobin, orysastrobin, picoxystrobin, pyraclostrobin, pyrametostrobin, pyraoxystrobin, ferbam, mancozeb, maneb, metiram, propineb, zineb, captafol, captan, fluoroimide, folpet, tolylfluanid, bordeaux mixture, copper oxide, mancopper, oxine-copper, nitrothal-isopropyl, edifenphos, iprobenphos, phosdiphen, tolclofos-methyl, anilazine, benthiavalicarb, blasticidin-S, chloroneb, chloro-tha-lonil, cyflufenamid, cymoxanil, cyclobutrifluram, diclocymet, diclomezine, dicloran, diethofencarb, dimethomorph, flumorph, dithianon, ethaboxam, etridiazole, famoxadone, fenamidone, fenoxanil, ferimzone, fluazinam, flumetylsulforim, fluopicolide, fluoxytioconazole, flusulfamide, fluxapyroxad,-fenhexamid, fosetyl-aluminium, hymexazol, iprovalicarb, cyazofamid, methasulfocarb, metrafenone, pencycuron, phthalide, polyoxins, propamocarb, pyribencarb, proquinazid, pyroquilon, pyriofenone, quinoxyfen, quintozene, tiadinil, triazoxide, tricyclazole, triforine, validamycin, valifenalate, zoxamide, mandipropamid, flubeneteram, isopyrazam, sedaxane, benzovindiflupyr, pydiflumetofen, 3-difluoromethyl-1-methyl-1H-pyrazole-4-carboxylic acid (3′,4′,5′-trifluoro-biphenyl-2-yl)-amide, isoflucypram, isotianil, dipymetitrone, 6-ethyl-5,7-dioxo-pyrrolo [4,5][1,4]dithiino[1,2-c]isothiazole-3-carbonitrile, 2-(difluoromethyl)-N-[3-ethyl-1,1-dimethyl-indan-4-yl]pyridine-3-carboxamide, 4-(2,6-difluorophenyl)-6-methyl-5-phenyl-pyridazine-3-carbonitrile, (R)-3-(difluoromethyl)-1-methyl-N-[1,1,3-trimethylindan-4-yl]pyrazole-4-carboxamide, 4-(2-bromo-4-fluoro-phenyl)-N-(2-chloro-6-fluoro-phenyl)-2,5-dimethyl-pyrazol-3-amine, 4-(2-bromo-4-fluorophenyl)-N-(2-chloro-6-fluorophenyl)-1, 3-dimethyl-1H-pyrazol-5-amine, fluindapyr, coumethoxystrobin (jiaxiangjunzhi), Ivbenmixianan, dichlobentiazox, mandestrobin, 3-(4,4-difluoro-3,4-dihydro-3,3-dimethylisoquinolin-1-yl) quinolone, 2-[2-fluoro-6-[(8-fluoro-2-methyl-3-quinolyl)oxy]phenyl]propan-2-ol, oxathiapiprolin, tert-butyl N-[6-[[(1-methyltetrazol-5-yl)-phenyl-methylene]amino]oxymethyl]-2-pyridyl]carbamate, pyraziflumid, inpyrfluxam, trolprocarb, mefentrifluconazole, ipfentrifluconazole, 2-(difluoromethyl)-N-[(3R)-3-ethyl-1,1-dimethyl-indan-4-yl]pyridine-3-carboxamide, N′-(2,5-dimethyl-4-phenoxy-phenyl)-N-ethyl-N-methyl-formamidine, N′-[4-(4,5-dichlorothiazol-2-yl)oxy-2,5-dimethyl-phenyl]-N-ethyl-N-methyl-formamidine, [2-[3-[2-[1-[2-[3,5-bis (difluoromethyl) pyrazol-1-yl]acetyl]-4-piperidyl]thiazol-4-yl]-4,5-dihydroisoxazol-5-yl]-3-chloro-phenyl]methanesulfonate, but-3-ynyl N-[6-[[(Z)-[(1-methyltetrazol-5-yl)-phenyl-methylene]amino]oxymethyl]-2-pyridyl]carbamate, methyl N-[5-[4-(2,4-dimethylphenyl)triazol-2-yl]-2-methyl-phenyl]methyl]carbamate, 3-chloro-6-methyl-5-phenyl-4-(2,4,6-trifluorophenyl)pyridazine, pyridachlometyl, 3-(difluoromethyl)-1-methyl-N-[1,1,3-trimethylindan-4-yl]pyrazole-4-carboxamide, 1-[2-[1-(4-chlorophenyl) pyrazol-3-yl]oxymethyl]-3-methyl-phenyl]-4-methyl-tetrazol-5-one, 1-methyl-4-[3-methyl-2-[[2-methyl-4-(3,4,5-trimethylpyrazol-1-yl)phenoxy]methyl]phenyl]tetrazol-5-one, aminopyrifen, ametoctradin, amisulbrom, penflufen, (Z,2E)-5-[1-(4-chlorophenyl) pyrazol-3-yl]oxy-2-methoxyimino-N,3-dimethyl-pent-3-enamide, florylpicoxamid, fenpicoxamid, metarylpicoxamid, tebufloquin, ipflufenoquin, quinofumelin, isofetamid, ethyl 1-[[4-[[2-(trifluoromethyl)-1,3-dioxolan-2-yl]methoxy]phenyl]methyl]pyrazole-3-carboxylate (may be prepared from the methods described in WO 2020/056090), ethyl 1-[[4-[(Z)-2-ethoxy-3,3,3-trifluoro-prop-1-enoxy]phenyl]methyl]pyrazole-3-carboxylate (may be prepared from the methods described in WO 2020/056090), methyl N-[4-[1-(4-cyclopropyl-2,6-difluoro-phenyl) pyrazol-4-yl]-2-methyl-phenyl]methyl]carbamate (may be prepared from the methods described in WO 2020/097012), methyl N-[4-[1-(2,6-difluoro-4-isopropyl-phenyl) pyrazol-4-yl]-2-methyl-phenyl]methyl]carbamate (may be prepared from the methods described in WO 2020/097012), 6-chloro-3-(3-cyclopropyl-2-fluoro-phenoxy)-N-[2-(2,4-dimethylphenyl)-2,2-difluoro-ethyl]-5-methyl-pyridazine-4-carboxamide (may be prepared from the methods described in WO 2020/109391), 6-chloro-N-[2-(2-chloro-4-methyl-phenyl)-2,2-difluoro-ethyl]-3-(3-cyclopropyl-2-fluoro-phenoxy)-5-methyl-pyridazine-4-carboxamide (may be prepared from the methods described in WO 2020/109391), 6-chloro-3-(3-cyclopropyl-2-fluoro-phenoxy)-N-[2-(3,4-dimethylphenyl)-2,2-difluoro-ethyl]-5-methyl-pyridazine-4-carboxamide (may be prepared from the methods described in WO 2020/109391), N-[2-[2,4-dichloro-phenoxyphenyl]-3-(difluoromethyl)-1-methyl-pyrazole-4-carboxamide, N-[2-[2-chloro-4-(trifluoromethyl)phenoxy]phenyl]-3-(difluoromethyl)-1-methyl-pyrazole-4-carboxamide, benzothiostrobin, phenamacril, 5-amino-1,3,4-thiadiazole-2-thiol zinc salt (2:1), fluopyram, flufenoxadiazam, flutianil, fluopimomide, pyrapropoyne, picarbutrazox, 2-(difluoromethyl)-N-(3-ethyl-1,1-dimethyl-indan-4-yl)pyridine-3-carboxamide, 2-(difluoromethyl)-N-((3R)-1, 1, 3-trimethylindan-4-yl)pyridine-3-carboxamide, 4-[[6-[2-(2,4-difluorophenyl)-1,1-difluoro-2-hydroxy-3-(1,2,4-triazol-1-yl) propyl]-3-pyridyl]oxy]benzonitrile, metyltetraprole, 2-(difluoromethyl)-N-((3R)-1, 1, 3-trimethylindan-4-yl)pyridine-3-carboxamide, a-(1, 1-dimethylethyl)-a-[4′-(trifluoromethoxy) [1,1′-biphenyl]-4-yl]-5-pyrimidinemethanol, fluoxapiprolin, enoxastrobin, methyl (Z)-3-methoxy-2-[2-methyl-5-[4-(trifluoromethyl)triazol-2-yllphenoxy]prop-2-enoate, methyl (Z)-3-methoxy-2-[2-methyl-5-(4-propyltriazol-2-yl)phenoxy]prop-2-enoate, methyl (Z)-2-[5-(3-isopropylpyrazol-1-yl)-2-methyl-phenoxy]-3-methoxy-prop-2-enoate, methyl (Z)-3-methoxy-2-[2-methyl-5-(3-propylpyrazol-1-yl)phenoxy]prop-2-enoate, methyl (Z)-3-methoxy-2-[2-methyl-5-[3-(trifluoromethyl) pyrazol-1-yl]phenoxy]prop-2-enoate (these compounds may be prepared from the methods described in WO2020/079111), methyl (Z)-2-(5-cyclohexyl-2-methyl-phenoxy)-3-methoxy-prop-2-enoate, methyl (Z)-2-(5-cyclopentyl-2-methyl-phenoxy)-3-methoxy-prop-2-enoate (these compounds may be prepared from the methods described in WO2020/193387), 4-[6-[2-(2,4-difluorophenyl)-1,1-difluoro-2-hydroxy-3-(1,2,4-triazol-1-yl) propyl]-3-pyridyl]oxy]benzonitrile, 4-[6-[2-(2,4-difluorophenyl)-1,1-difluoro-2-hydroxy-3-(5-sulfanyl-1,2,4-triazol-1-yl) propyl]-3-pyridyl]oxy]benzonitrile, 4-[6-[2-(2,4-difluorophenyl)-1,1-difluoro-2-hydroxy-3-(5-thioxo-4H-1,2,4-triazol-1-yl) propyl]-3-pyridyl]oxy]benzonitrile, trinexapac, coumoxystrobin, zhongshengmycin, thiodiazole copper, zinc thiazole, amectotractin, iprodione, seboctylamine; N′-[5-bromo-2-methyl-6-[(1S)-1-methyl-2-propoxy-ethoxy]-3-pyridyl]-N-ethyl-N-methyl-formamidine, N′-[5-bromo-2-methyl-6-[(1R)-1-methyl-2-propoxy-ethoxy]-3-pyridyl]-N-ethyl-N-methyl-formamidine, N′-[5-bromo-2-methyl-6-(1-methyl-2-propoxy-ethoxy)-3-pyridyl]-N-ethyl-N-methyl-formamidine, N′-[5-chloro-2-methyl-6-(1-methyl-2-propoxy-ethoxy)-3-pyridyl]-N-ethyl-N-methyl-formamidine, N′-[5-bromo-2-methyl-6-(1-methyl-2-propoxy-ethoxy)-3-pyridyl]-N-isopropyl-N-methyl-formamidine (these compounds may be prepared from the methods described in WO2015/155075); N′-[5-bromo-2-methyl-6-(2-propoxypropoxy)-3-pyridyl]-N-ethyl-N-methyl-formamidine (this compound may be prepared from the methods described in IPCOM 000249876D); N-isopropyl-N′-[5-methoxy-2-methyl-4-(2,2,2-trifluoro-1-hydroxy-1-phenyl-ethyl)phenyl]-N-methyl-formamidine, N′-[4-(1-cyclopropyl-2,2,2-trifluoro-1-hydroxy-ethyl)-5-methoxy-2-methyl-phenyl]-N-isopropyl-N-methyl-formamidine (these compounds may be prepared from the methods described in WO2018/228896); N-ethyl-N′-[5-methoxy-2-methyl-4-[(2-trifluoromethyl) oxetan-2-yl]phenyl]-N-methyl-formamidine, N-ethyl-N′-[5-methoxy-2-methyl-4-[(2-trifuoromethyl)tetrahydrofuran-2-yl]phenyl]-N-methyl-formamidine (these compounds may be prepared from the methods described in WO2019/110427); N-[(1R)-1-benzyl-3-chloro-1-methyl-but-3-enyl]-8-fluoro-quinoline-3-carboxamide, N-[(1S)-1-benzyl-3-chloro-1-methyl-but-3-enyl]-8-fluoro-quinoline-3-carboxamide, N-[(1R)-1-benzyl-3,3,3-trifluoro-1-methyl-propyl]-8-fluoro-quinoline-3-carboxamide, N-[(1S)-1-benzyl-3,3,3-trifluoro-1-methyl-propyl]-8-fluoro-quinoline-3-carboxamide, N-[(1R)-1-benzyl-1,3-dimethyl-butyl]-7,8-difluoro-quinoline-3-carboxamide, N-[(1S)-1-benzyl-1,3-dimethyl-butyl]-7,8-difluoro-quinoline-3-carboxamide, 8-fluoro-N-[(1R)-1-[(3-fluorophenyl)methyl]-1,3-dimethyl-butyl]quinoline-3-carboxamide, 8-fluoro-N-[(1S)-1-[(3-fluorophenyl)methyl]-1,3-dimethyl-butyl]quinoline-3-carboxamide, N-[(1R)-1-benzyl-1,3-dimethyl-butyl]-8-fluoro-quinoline-3-carboxamide, N-[(1S)-1-benzyl-1,3-dimethyl-butyl]-8-fluoro-quinoline-3-carboxamide, N-((1R)-1-benzyl-3-chloro-1-methyl-but-3-enyl)-8-fluoro-quinoline-3-carboxamide, N-((1S)-1-benzyl-3-chloro-1-methyl-but-3-enyl)-8-fluoro-quinoline-3-carboxamide (these compounds may be prepared from the methods described in WO2017/153380); 1-(6,7-dimethylpyrazolo[1,5-a]pyridin-3-yl)-4,4,5-trifluoro-3,3-dimethyl-isoquinoline, 1-(6,7-dimethylpyrazolo[1,5-a]pyridin-3-yl)-4,4,6-trifluoro-3,3-dimethyl-isoquinoline, 4,4-difluoro-3,3-dimethyl-1-(6-methylpyrazolo[1,5-a]pyridin-3-yl) isoquinoline, 4,4-difluoro-3,3-dimethyl-1-(7-methylpyrazolo[1,5-a]pyridin-3-yl) isoquinoline, 1-(6-chloro-7-methyl-pyrazolo[1,5-a]pyridin-3-yl)-4,4-difluoro-3,3-dimethyl-isoquinoline (these compounds may be prepared from the methods described in WO 2017/025510); 1-(4,5-dimethylbenzimidazol-1-yl)-4,4,5-trifluoro-3,3-dimethyl-isoquinoline, 1-(4,5-dimethylbenzimidazol-1-yl)-4,4-difluoro-3,3-dimethyl-isoquinoline, 6-chloro-4,4-difluoro-3,3-dimethyl-1-(4-methylbenzimidazol-1-yl) isoquinoline, 4,4-difluoro-1-(5-fluoro-4-methyl-benzimidazol-1-yl)-3,3-dimethyl-isoquinoline, 3-(4,4-difluoro-3,3-dimethyl-1-isoquinolyl)-7,8-dihydro-6H-cyclopenta[e]benzimidazole (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide, N,2-dimethoxy-N-[[4-[5-(trifluoromethyl)-1,2,4-oxadiazol-3-yl]phenyl]methyl]propanamide, N-ethyl-2-methyl-N-[[4-[5-(trifluoromethyl)-1,2,4-oxadiazol-3-yl]phenyl]methyl]propanamide, 1-methoxy-3-methyl-1-[[4-[5-(trifluoromethyl)-1,2,4-oxadiazol-3-yl]phenyl]methyl]urea, 1,3-dimethoxy-1-[[4-[5-(trifluoromethyl)-1,2,4-oxadiazol-3-yl]phenyl]methyl]urea, 3-ethyl-1-methoxy-1-[[4-[5-(trifluoromethyl)-1,2,4-oxadiazol-3-yl]phenyl]methyl]urea, N-[[4-[5-(trifluoromethyl)-1,2,4-oxadiazol-3-yl]phenyl]methyl]propanamide, 4,4-dimethyl-2-[[4-[5-(trifluoromethyl)-1,2,4-oxadiazol-3-yl]phenyl]methyl]isoxazolidin-3-one, 5,5-dimethyl-2-[[4-[5-(trifluoromethyl)-1,2,4-oxadiazol-3-yl]phenyl]methyl]isoxazolidin-3-one, ethyl 1-[[4-[5-(trifluoromethyl)-1,2,4-oxadiazol-3-yl]phenyl]methyl]pyrazole-4-carboxylate, N,N-dimethyl-1-[[4-[5-(trifluoromethyl)-1,2,4-oxadiazol-3-yl]phenyl]methyl]-1,2,4-triazol-3-amine. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl) propan-2-ol (this compound may be prepared from the methods described in WO 2017/029179); 2-[6-(4-bromophenoxy)-2-(trifluoromethyl)-3-pyridyl]-1-(1,2,4-triazol-1-yl) propan-2-ol (this compound may be prepared from the methods described in WO 2017/029179); 3-[2-(1-chlorocyclopropyl)-3-(2-fluorophenyl)-2-hydroxy-propyl]imidazole-4-carbonitrile (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile (this compound may be prepared from the methods described in WO 2016/156290); (4-phenoxyphenyl)methyl 2-amino-6-methyl-pyridine-3-carboxylate (this compound may be prepared from the methods described in WO 2014/006945); 2,6-Dimethyl-1H,5H-[1,4]dithiino[2,3-c:5,6-c′]dipyrrole-1,3,5,7 (2H,6H)-tetrone (this compound may be prepared from the methods described in WO 2011/138281); N-methyl-4-[5-(trifluoromethyl)-1,2,4-oxadiazol-3-yl]benzenecarbothioamide; N-methyl-4-[5-(trifluoromethyl)-1,2,4-oxadiazol-3-yl]benzamide; (Z,2E)-5-[1-(2,4-dichlorophenyl) pyrazol-3-yl]oxy-2-methoxyimino-N,3-dimethyl-pent-3-enamide (this compound may be prepared from the methods described in WO 2018/153707); N′-(2-chloro-5-methyl-4-phenoxy-phenyl)-N-ethyl-N-methyl-formamidine; N′-[2-chloro-4-(2-fluorophenoxy)-5-methyl-phenyl]-N-ethyl-N-methyl-formamidine (this compound may be prepared from the methods described in WO 2016/202742); 2-(difluoromethyl)-N-[(3S)-3-ethyl-1,1-dimethyl-indan-4-yl]pyridine-3-carboxamide (this compound may be prepared from the methods described in WO 2014/095675); (5-methyl-2-pyridyl)-[4-[5-(trifluoromethyl)-1,2,4-oxadiazol-3-yl]phenyl]methanone, (3-methylisoxazol-5-yl)-[4-[5-(trifluoromethyl)-1,2,4-oxadiazol-3-yl]phenyl]methanone (these compounds may be prepared from the methods described in WO 2017/220485); 2-oxo-N-propyl-2-[4-[5-(trifluoromethyl)-1,2,4-oxadiazol-3-yl]phenyl]acetamide (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate (this compound may be prepared from the methods described in WO 2018/158365); 2,2-difluoro-N-methyl-2-[4-[5-(trifluoromethyl)-1,2,4-oxadiazol-3-yl]phenyl]acetamide, N-[(E)-methoxyiminomethyl]-4-[5-(trifluoromethyl)-1,2,4-oxadiazol-3-yl]benzamide, N-[(Z)-methoxyiminomethyl]-4-[5-(trifluoromethyl)-1,2,4-oxadiazol-3-yl]benzamide, and N-[N-methoxy-C-methyl-carbonimidoyl]-4-[5-(trifluoromethyl)-1,2,4-oxadiazol-3-yl]benzamide (these compounds may be prepared from the methods described in WO 2018/202428).

In another embodiment, the present invention provides a corn plant comprising elite event MZIR260, wherein the corn plant is useful for control of Spodoptera spp. and/or Diatraea spp. insect pests. In further embodiments, the corn plant is useful for control of Spodoptera frugiperda (fall armyworm) and, optionally, Diatraea saccharalis (sugarcane borer). In another embodiment, the corn plant is useful for control of Spodoptera frugiperda (fall armyworm), Diatraea saccharalis (sugarcane borer), Mythimna separata (oriental armyworm), Spodoptera litura (common cutworm/oriental leafworm), and Ostrinia furnacalis (Asian corn borer). Another embodiment of the invention is a method of controlling insect pests, comprising planting a corn plant comprising elite event MZIR260 in a field. The field may comprise at least one MZIR260 corn plant, at least 50% MZIR260 corn plants, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% MZIR260 corn plants. In some embodiments, the field comprises refuge corn plants (e.g., at least 5% or at least 10% of the field) that do not comprise elite event MZIR260, but which may include other events such as those for herbicide tolerance. The present invention also provides a use of a corn plant comprising elite event MZIR260 for controlling insect pests in a field. The insect pests may be Spodoptera spp. and/or Diatraea spp. The insect pests may further be Spodoptera frugiperda (fall armyworm) and/or Diatraea saccharalis (sugarcane borer).

Another embodiment of the invention is a recombinant sequence, which comprises a maize chromosomal target site located on chromosome 2 between nucleotide coordinates 140849156 and 140851379 in the reference B 73 corn genome and a heterologous nucleic acid. The heterologous nucleic acid may be introduced at the maize chromosomal target site by targeted insertion. A further embodiment is a recombinant nucleic acid molecule of chromosome 2 comprising a heterologous nucleic acid sequence inserted on chromosome 2, wherein the heterologous nucleic acid sequence comprises SEQ ID NO: 1, SEQ ID NO: 2, SEQ ID NO: 3, SEQ ID NO: 4, SEQ ID NO: 5, or SEQ ID NO: 6. A preferred embodiment is a recombinant nucleic acid molecule comprising a heterologous nucleic acid inserted on chromosome 2, wherein the heterologous nucleic acid is set forth as SEQ ID NO:. 5.

Another embodiment of the invention is a method of making a transgenic maize plant comprising inserting a heterologous nucleic acid at a position on chromosome 2, wherein the heterologous nucleic acid comprises the nucleotide sequence set forth in SEQ ID NO: 1, SEQ ID NO: 2, SEQ ID NO: 3, SEQ ID NO: 4, SEQ ID NO: 5, or SEQ ID NO: 6. A preferred embodiment is a method of making a transgenic maize plant, wherein the heterologous nucleic acid comprises the nucleotide sequence set forth in SEQ ID NO: 5.

In some aspects, the disclosure provides methods of modifying elite event MZIR260, e.g., in a cell or plant. In some embodiments, the modification is a deletion, an insertion (e.g., of a heterologous nucleic acid sequence), a substitution, a duplication, or inversion, or a combination thereof. In some embodiments, the modification comprises deletion of a portion or all of a selectable marker coding sequence present in the nucleic acid molecule, e.g., a PMI coding sequence. In some embodiments, the modification is introduced using a nuclease, such as a CRISPR-Cas nuclease, a Zinc finger nuclease, a meganuclease, a TAL effector nuclease (TALEN), or a combination thereof.

In some embodiments, the modification is made in a host cell or plant of the disclosure, e.g., a maize cell or maize plant, to produce a modified transgenic cell or modified transgenic plant. In some embodiments, the modification is made by expressing the nuclease in the host cell or plant (e.g., by transforming the host cell or plant with an expression cassette encoding the nuclease or by crossing the plant with another plant containing such an expression cassette). In some embodiments, the modification is made by directly introducing the nuclease into the host cell or plant, e.g., using reagents that transfer the nuclease into the host cell or plant such as through physical methods such as biolistics/particle bombardment, protoplast transfection, nanoparticle-mediated delivery, aerosol bean injection, or whisker-mediated delivery. In some embodiments, the method further comprises producing a plant from the modified transgenic host cell to produce a modified transgenic plant. In some embodiments, the method further comprises selfing or crossing the modified transgenic plant with another plant for at least one generation (e.g., one, two, three, four or more generations), thereby producing a modified transgenic progeny plant. In some embodiments, the disclose provides such a modified transgenic cell, modified transgenic plant, or modified transgenic progeny plant, e.g., produced by a method herein.

In certain embodiments, the nucleic acid modification is affected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Non-limiting examples of methods of using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is affected by a meganuclease, which are endodeoxyribonucleases characterized by a large recognition site (double-stranded DNA sequences of 12 to 40 base pairs). Non-limiting examples of methods of using meganucleases can be found in U.S. Pat. Nos. 8,163,514; 8,133,697; 8,021,867; 8,119,361; 8,119,381; 8,124,369; and 8,129,134

In certain embodiments, the nucleic acid modification is affected by a CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a Cas nuclease can be programmed by an RNA guide (gRNA) to recognize a specific nucleic acid target, in other words the Cas nuclease can be recruited to a specific nucleic acid target locus of interest using said short RNA guide.

In general, the CRISPR/Cas or CRISPR system as used herein refers collectively to the elements involved in the expression of or directing the activity of CRISPR-associated (“Cas”) nuclease, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a″spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas nuclease is Cas12a).

The CRISPR-Cas nuclease can be any such nuclease known in the art, such as a Cas9, Cas12a, Cas12b, Cas12i, Cas13a (formerly referred to as C2c2), C2c3, Cas13b or a modified version of any of the foregoing. CRISPR-Cas nucleases are well known in the art (see, e.g., Dong et al. Efficient Targeted Mutagenesis Mediated by CRISPR-Cas12a Ribonucleoprotein Complexes in Maize. Front. Genome Ed. (2021), vol. 3, article 670529; Wei et al. TALEN or Cas9-Rapid, Efficient and Specific Choices for Genome Modifications. J. of Genetics and Genomics (2013), vol. 40, pp. 281-289; Sedeek et al. Plant Genome Engineering for Targeted Improvement of Crop Traits. Frontiers in Plant Science (2019), vol. 10, article 114; and Zhang et al. Applications and potential of genome editing in crop improvement. Genome Biology (2018), vol. 19, article 210).

### EXAMPLES

The invention will be further described by reference to the following detailed examples. These examples are provided for purposes of illustration only, and are not intended to be limiting unless otherwise specified. Standard recombinant DNA and molecular cloning techniques used here are well known in the art and are described by Ausubel (ed.), Current Protocols in Molecular Biology, John Wiley and Sons, Inc. (1994); J. Sambrook, et al., Molecular Cloning: A Laboratory Manual, 3d Ed., Cold Spring Harbor, NY: Cold Spring Harbor Laboratory Press (2001); and by T. J. Silhavy, M. L. Berman, and L. W. Enquist, Experiments with Gene Fusions, Cold Spring Harbor Laboratory, Cold Spring Harbor, NY (1984).

### Example 1. Construct Selection, Transformation and Event Selection

Construct 24795 was constructed containing the promoter, CDS and terminator for each of eCry1Gb.1Ig and PMI as listed in Table 2. These genetic elements were synthesized and ligated into the binary vector through a restriction enzyme based cloning method.

TABLE 2
Cassette assembly

| Construct | Cassette | | | |
|---|---|---|---|---|
| ID | position | Promoter | CDS | Terminator |
| 24795 | 1 | SoUbi4-02 | eCry1Gb.1lg-03 | ZmUbi361-05 |
| | 2 | Ubi1-43 | pmi-15 | Ubi1-04 |

Transformation of Zea mays to produce genetically modified maize was accomplished using immature embryos via Agrobacterium tumefaciens-mediated transformation, as described in Zhong et al. (2018) (Advances in Agrobacterium-mediated Maize Transformation. In: Lagrimini L. (eds) Maize. Methods in Molecular Biology, vol 1676. Humana Press, New York, NY). A. tumefaciens strain LBA4404 (recA-) harboring disarmed pTi plasmid pA L4404 and helper plasmid pVGW7 was used for maize transformation. Detailed information about the pA L4404 and pVGW7 plasmids is described by Hoekema et al. (Nature. (1983) 303:179-189), Ishida et al. (Nat Biotechnol (1996) 14:745-750) and Imayama et al. (U.S. Pat. No. 10,266,835). A. tumefaciens strain L BA 4404 (recA) containing individual binary vectors was prepared as described by Li et al. (Plant Physiol (2003) 133:736-47). For maize transformation, immature embryos from greenhouse grown maize inbred line NP2222 were harvested approximately 9 days after pollination and used as explants (Zhong et al., 2018). Immature embryo isolation, Agrobacterium inoculation and co-cultivation of Agrobacterium with the immature embryos were performed as described in Zhong et al. (2018) using the bulk extraction method described therein. Using this method, genetic elements within the left and right border regions of the transformation plasmid were efficiently transferred and integrated into the genome of the plant cell, while genetic elements outside these border regions were not transferred.

Transformed tissues and putative transgenic events were regenerated and rooted as described earlier (Zhong et al., 2018) using media with mannose selection for events containing a phosphomannose isomerase (PMI) selectable marker (Negrotto et al., (2000) Plant Cell Rep. 19:789-803.). Regenerated plantlets were tested for the presence of eCry1Gb.1Ig-03 and pmi-15 by real-time TAQMAN® PCR analysis developed by Ingham et al. (Biotechniques 31 (1): 132-4, 136-40, 2001). Plants with putative single copy for all transgenes and without transformation plasmid backbone contamination were transferred to the greenhouse for further analysis and seed setting.

As shown in Table 3, a large-scale corn transformation effort was undertaken in which embryos were transformed to generate 1064 any-copy events. An any-copy event may contain multiple T-DNA insertions at multiple locations in the genome and/or whole or partial duplications and/or T-DNA rearrangements at a given insertion site. Two hundred sixty-nine (269) of these events were putatively determined by primary TaqMan® PCR analysis to have a single insertion, with no backbone DNA. After Events were sent to the greenhouse, secondary TaqMan® PCR and ELISA analyses were conducted to confirm the single copy, plasmid backbone-free events with desirable levels of expression for the trait protein. Sixty-four (64) events were selected for additional molecular characterization. This molecular characterization included verification of coding sequences to eliminate events with altered protein coding sequences, flanking sequence assessment, and confirmation of the absence of plasmid backbone. Flanking sequence assessment involved flanking sequence recovery, such that the genomic location of the transgene insertion site could be determined and characterized, confirming that the transgene insertion site met quality criteria. To advance, an event was free of the plasmid backbone, contained one and only copy of the intact T-DNA insertion, and had no genetic rearrangements. This molecular characterization data together with greenhouse seed production was used to select events to go to the field nursery. Of the events evaluated, twenty-four (24) backbone-free, intact (no genetic rearrangement) single-copy events were selected for field trials.

TABLE 3
Event selection

| T0 Event Selection | Events | % |
|---|---|---|
| Any-copy events evaluated | 1064 | 100 |
| Events that passed 1° TaqM an ® and sent to | 269 | 25.3 |
| greenhouse | | |
| Events selected based on 2° TaqMan ® confirmation | 155 | 14.6 |
| Events selected based on ELISA protein expression | 64 | 6 |
| in leaves | | |
| Events that passed CDS coding sequence verification | 62 | 5.8 |
| to eliminate events with altered amino acid sequences | | |
| Events that passed flanking sequence assessment to | 32 | 3 |
| avoid genic regions | | |
| Events for which sufficient T1 and F1 seed was | 27 | 2.5 |
| obtained in greenhouse | | |
| Events planted in field trials | 24 | 2.3 |

The field trials typically comprised a hybrid line from each event tested, with at least fifteen (15) plants of each hybrid per plot, three (3) replicates per location, and at least four (4) locations evaluated. For the field trials, fall armyworm damage was assessed using the O-9 Davis scale. All field trials included positive controls of transgenic event MIR162 (U.S. Pat. No. 9,752,198). All field trials also included a negative control which was non-transgenic corn. Events were also evaluated for agronomic performance, including yield. Events which performed the best compared to the positive and negative controls were selected for advancement.

Following two years of field trials, events were selected based on trait and agronomic performance, using assays similar to those described in Examples 9 and 10. After two seasons of additional field trials, events were selected for further evaluation, and finally 10 events were evaluated in extensive event selection trials.

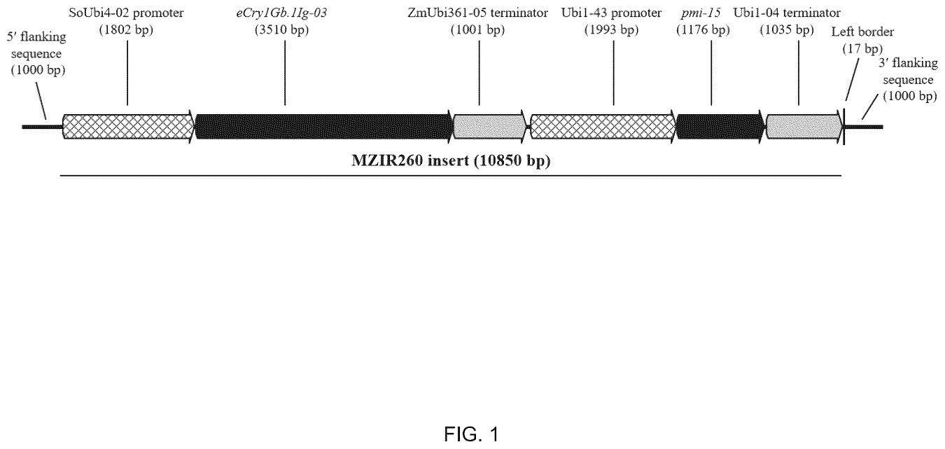

Based on the combination of all of the trait efficacy trials, agronomic trials, molecular characterization, and level of protein expression of eCry1Gb.1Ig, MZIR260 was identified as an elite event. The superior performance of plants comprising the MZIR260 event is due to the intactness of the transgene itself and to the transgene insertion at a specific location in the corn genome which supports adequate expression for high efficacy of the trait gene encoded on the transgene, and which further does not have any significant negative impacts on plant performance. Additionally, the high agronomic performance and trait efficacy of all MZIR260 hybrids produced from multiple genetic backgrounds further showcased the elite quality of the MZIR260 event. A gain, this superiority was likely due to the combination of the exact genomic location of the transgene and characteristics of the transgene insertion as discussed supra. FIG. 1 illustrates the transgene of MZIR260 inserted into the corn genome.

### Example 2. TaqMan® Assay for MZIR260

A real-time, MZIR260-specific polymerase chain reaction (PCR) method was developed to detect and quantify MZIR260 deoxyribonucleic acid (DNA) extracted from seed, grain and other plant material samples. The method consists of a maize-specific PCR method as a reference and an event-specific PCR method for detection and quantification of MZIR260 maize DNA. This method can be used to determine the relative content of event MZIR260 maize DNA in proportion to total maize DNA in samples.

For specific detection of MZIR260 maize genomic DNA, two specific primers (SEQ ID NO: 11 and SEQ ID NO: 12) were used to amplify an 88-bp fragment of the region that spans the 3′ insert-to-plant genome junction (SEQ ID NO: 10). A control primer set and probe (for example, to an endogenous maize gene such as the alcohol dehydrogenase 1 (adh1) gene) may be used as a positive control. The amount of PCR product is determined during each cycle in real-time by measuring the fluorescence produced by an MZIR260-specific oligonucleotide probe labeled with 6-FAM™ as a reporter dye at its 5′ end and M GB-NFQ at its 3′ end (SEQ ID NO: 13). The primers and probe are shown in Table 4. Table 5 lists additional primers that can be used to detect the MZIR260 event and gene-specific targets in the MZIR260 insert.

TABLE 4
Elite Event M ZIR 260 Detection by TaqMan® PCR

| | designed for 3′ genome-insert | | |
|---|---|---|---|
| | junction | | |
| Sequence | Descrip- | SEQ ID | |
| Name | tion | NO: | Sequence 5′ to 3′ |
| M ZIR 260- | Forward | 11 | GTTATTAAGTTGTCT |
| esqPCR-F | primer | | AAGCGTCAATTTGTT |
| M ZIR 260- | Reverse | 12 | CGCCATAGTGTACTT |
| esqPCR-R | primer | | GACATCTTATGT |
| M ZIR 260- | Probe | 13 | CACCACAATATAAAA |
| esqPCR-Pr | | | AG |

TABLE 5
Elite M ZIR 260 Detection by TaqMan® and/
or gel-based PCR of (1) Event M ZIR 260
(2) Gene-specific targets in M ZIR 260 Insert

| Target/Assay | Descrip- | SEQ ID | |
|---|---|---|---|
| type | tion | NO | Sequence 5′ to 3′ |
| eCry1Gb.1lg-03 | Forward | 14 | CTCCAGGCTGAAGAACAGCAT |
| (gene-specific)- | primer | | |
| TaqMan® | Reverse | 15 | GCCCAGGCCGATGTTGTA |
| | primer | | |
| | Probe | 16 | ACTACACGAACCACTGCGTGCGCTT |
| 5′ Genome-Insert | Forward | 17 | CAGTCATAGATGATGGGTGATTCAA |
| Junction (Event- | primer | | |
| specific) | Reverse | 18 | AGCTAATTAGGGAAGCGGCCTA |
| TaqMan® | primer | | |
| | Probe | 19 | TAGCCTAACGGTGTTGACTA |
| 3′ Genome-Insert | M ZIR 260- | 20 | CGTGAGCCTTTAGCAACTAGCTAGA |
| Junction (Event- | ESQL-F | | |
| specific) Gel- | M ZIR 260- | 21 | GCGCCATAGTGTACTTGACATCTTA |
| based PCR | ESQL-R | | |
| pmi-15 | forward | 22 | CCGGGTGAATCAGCGTTT |
| | primer | | |
| | reverse | 23 | GCCGTGGCCTTTGACAGT |
| | primer | | |
| | probe | 24 | TGCCGCCAACGAATCACCGG |

It is recognized that other primer combinations could be used to detect MZIR260 junction sequences. For example, to detect the 5′ junction sequence (SEQ ID NO: 1), a first primer may be SEQ ID NO: 17, or the complement thereof, and a second primer may be SEQ ID NO: 19, or the complement thereof. A skilled person of the art would recognize that a first primer comprising a sequence from the 5′ flanking sequence (SEQ ID NO: 8, or a complement thereof) and a second primer comprising a sequence from within the transgene (SEQ ID NO: 7, or a complement thereof) can function as a pair in a PCR reaction to produce an amplicon that comprises the 5′ junction sequence, and this amplicon may be detected by a probe, for example, SEQ ID NO: 19. Similarly, to detect the 3′ junction sequence (SEQ ID NO: 2), a first primer may be SEQ ID NO: 20, or the complement thereof, and a second primer may be SEQ ID NO: 21 or a complement thereof. A skilled person of the art would recognize that a first primer comprising a sequence from the 3′ flanking sequence (SEQ ID NO: 9, or a complement thereof) and a second primer comprising a sequence from within the transgene (SEQ ID NO: 7, or a complement thereof) can function as a pair in a PCR reaction to produce an amplicon that comprises the 3′ junction sequence, and this amplicon may be detected by a probe, for example. It is important to note that these junction sequences are novel to the invention described herein. Therefore, it is only with the present disclosure that a skilled person would know the junction sequences which are novel and unique to elite event MZIR260, and also have motivation to detect them.

TaqMan® analysis was essentially carried out as described in Ingham et al. (Biotechniques, 31:132-140, 2001) herein incorporated by reference. Genomic DNA from biological samples are prepared using methods known in the art.

TaqMan® PCR reactions were carried out either in 96- or 384-well plates. For the endogenous corn gene control, primers and probes were designed specific to the Zea mays alcohol dehydrogenase 1 (adh1) gene (Genbank accession no. AY 691949). It will be recognized by the skilled person that other corn genes can be used as endogenous controls.

The master mix for the maize adh1-specific and event MZIR260-specific TaqMan PCR is shown in the Table 6.

TABLE 6
Detection of M ZIR 260 by TaqM an ® PCR

| Components | Final concentration | |
|---|---|---|
| DNA | 5-50 | ng |
| JUMPSTART ™ Taq READYMIX ™* | 1X | |
| MZIR260 forward primer | 300 | nM |
| MZIR260 reverse primer | 300 | nM |
| MZIR260 probe | 100 | nM |
| adh1 forward primer | 300 | nM |
| adh1 reverse primer | 300 | nM |
| adh1 probe | 100 | nM |

| | |
|---|---|
| Nuclease-free water | as needed |

*supplemented with Sulforhodamine 101 and MgCl₂

PCR is performed in the Life Technologies QuantStudion™ Flex 7 Real-Time PCR instrument using the following amplification parameters: 10 min at 95° C., followed by 40 cycles of 15 s at 95° C. and 1 min at 60° C. It will be understood that cycle parameters can be optimized for temperature, time and/or cycle number. The PCR may be run on an ABI 7900HT, a GENEAMP™ PCR system 9700, or any other appropriate system. The post-run analysis was performed according to manufacturer's instructions. Both MZIR260 event-specific assay and gene specific assays could be run for real-timePCR analysis or in end-point PCR analysis. Results validated the above methodology, verifying it successfully identified the MZIR260 event in a biological sample derived from corn tissue, including seed, grain, and other plant material samples.

Both real-time TaqMan® analysis and end-point TaqMan® analysis could be used to determine zygosity status of event MZIR260 present in individual plants or seeds comprising elite event MZIR260. When end-point TaqMan® analysis would be used for Zygosity call, MZIR260 event specific primer/probe set could be coupled with a wild type allele primer/probe set in a duplex reaction. The wild type allele primer/probe set could be designed by a person skilled in the field based on the native insertion site sequence of MZIR260 event.

### Example 3-Mendelian Inheritance of MZIR260

Mendelian inheritance of the trait gene eCry1Gb.1Ig-03 and the selectable marker gene pmi-15 present in Event MZIR260 was assessed in three segregating generations (BC₂F₁, BC₃F₁, and F₂) of MZIR260 maize. Backcross (BC) generations for MZIR260 were produced by crossing a hemizygous MZIR260 maize parent (F1) to a nontransgenic recurrent parent (BDAX4608), and this backcrossing was repeated through several breeding cycles to yield the BC₂F₁ and BC₃F₁ generations. Positive hemizygous segregants, as determined by mannose resistance, were utilized in each backcross as the transgenic parent. In addition, the F1 generation was selfed to yield the F2 generation.

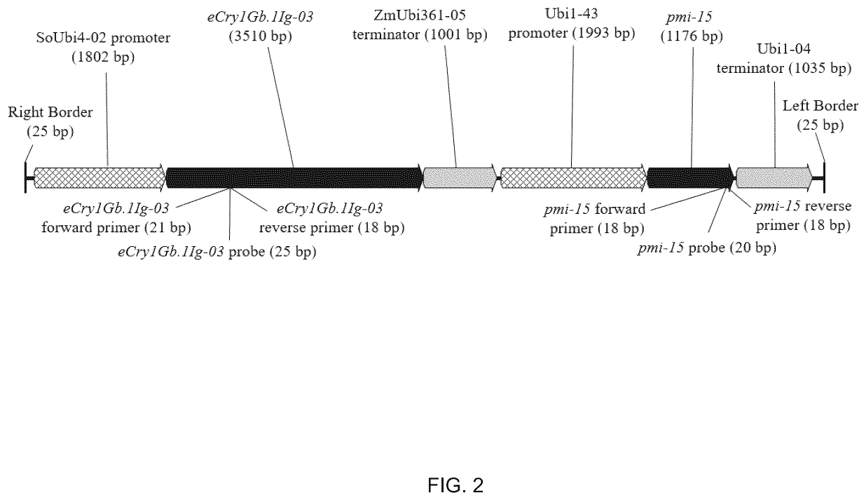

A total of 210, 218, and 132 plants were individually analyzed from BC₂F₁, BC₃F₁, and F2 generations, respectively, to determine copy number of eCry1Gb.1Ig-03 and pmi-15 by real-time quantitative PCR (qPCR) analysis (Ingham et al. 2001). For all plants, a control assay targeting the maize gene, adh1 (alcohol dehydrogenase 1), was used to confirm the presence of DNA in each reaction and to confirm that the real-time qPCR reagents were functioning as expected. Table 6 lists the primers and probes used to detect eCry1Gb.1Ig-03, pmi-15, and the maize endogenous gene adh1. FIG. 2 shows the locations of the eCry1Gb.1Ig-03 and pmi-15-specific primers and probes in the transferred DNA (T-DNA) of the transformation plasmid 24795 used to create Event MZIR260.

TABLE 6
Real-time qPCR Primers and Probes Used
for the Detection of
eCry1G b.11g-03, pmi-15, and adh1

| | SEQ | |
|---|---|---|
| | ID | |
| Target | NO: | Sequence 5′ to 3′ |
| eCry1Gb.1lg-03 | 14 | CTCCAGGCTGAAGAACAG |
| forward primer | | CAT |
| eCry1Gb.1lg-03 | 15 | GCCCAGGCCGATGTTGTA |
| reverse primer | | |
| eCry1Gb.1lg-03 | 16 | ACTACACGAACCACTGCG |
| probe | | TGCGCTT |
| pmi-15 forward | 22 | CCGGGTGAATCAGCGTTT |
| primer | | |
| pmi-15 reverse | 23 | GCCGTGGCCTTTGACAGT |
| primer | | |
| pmi-15 probe | 24 | TGCCGCCAACGAATCACCGG |
| adh1 forward | 25 | GAACGTGTGTTGGGTTTGCAT |
| primer | | |
| adh1 reverse | 26 | TCCAGCAATCCTTGCACCTT |
| primer | | |
| adh1 probe | 27 | TGCAGCCTAACCATGCGCAG |
| | | GGTA |

The following PCR cycling parameters are used for all assays: 10 min at 95° C., followed by 40 cycles of 15 s at 95° C. and 1 min at 60° C. It will be understood that cycle parameters can be optimized for temperature, time and/or cycle number.

The copy number for eCry1Gb.1Ig-03 and pmi-15 determined by real-time quantitative PCR (qPCR) from individual plants in the BC₂F₁ and BC₃F₁, and F₂ generations was used to calculate gene frequency and observed segregation ratios. The frequencies for eCry1Gb.1Ig-03 and pmi-15 were identical in each of the three generations, indicating that the two genes segregate as a single locus. The expected segregation ratio for eCry1Gb.1Ig-03 and pmi-15 in the BC₂F₁ and BC₃F₁ generations is 1:1 (i.e., 50% of the plants in each generation contain one copy of the transgenes and 50% of the plants in each generation do not contain the transgenes). The expected segregation ratio for eCry1Gb.1Ig-03 and pmi-15 in the F₂ generation is 1:2:1 (i.e., 25% of the plants contain two copies of the transgenes, 50% of the plants contain one copy of the transgenes, and 25% of the plants do not contain the transgenes).

The goodness-of-fit of the observed segregation ratios to the expected segregation ratios was tested by chi-square analysis (Strickberger 1976, Genetics, 2nd ed. New York: Macmillan Publishing Company. pp. 140-163). The chi-square critical value at α=0.05 is 3.84 for the BC₂F₁ and BC₃F₁ generations and 5.99 for the F₂ generation. The chi-square value was less than 3.84 for the BC₂F₁ and BC₃F₁ generations and less than 5.99 for the F₂ generation tested indicating that eCry1Gb.1Ig-03 and pmi-15 in MZIR260 maize are inherited in a predictable manner, according to Mendelian principles and consistent with insertion into a chromosome within the maize nuclear genome.

### Example 4. Analysis of Flanking DNA Sequence

The DNA sequence of the entire MZIR260 insert and 1000 base pairs (bp) of the maize genomic regions flanking the insert was determined to assess the intactness of the insert, the organization of the functional elements, and the presence of any rearrangements, deletions, and/or base pair changes within the MZIR260 insert. The MZIR260 insert sequence was compared to the T-DNA sequence in the transformation plasmid 24975 to determine if any changes had occurred during the transformation process.

Tissue Harvest and DNA Extraction. MZIR260 maize plants (approximately 100) were grown under greenhouse conditions for approximately twelve days, and afterwards moved to the dark and grown for three days at 25° C. to produce etiolated tissue for high molecular weight (HMW) DNA extraction. Leaf tissue was harvested and stored at −80° C. until ready for use. The leaf tissue was ground into a fine powder using a pre-chilled mortar and pestle, and liquid nitrogen. The ground tissue was immediately used for DNA extraction. The isolation of HMW genomic DNA (gDNA) fragments from plant nuclei and the shearing of the HMW gDNA embedded in agarose were performed at Bio S&T (Saint-Laurent, Quebec, Canada) using proprietary methods, as well as methods outlined in Zhang et al. (1995) Plant J 7:175-84. Briefly, nuclei were prepared using 7.7 grams of leaf tissue. The isolated nuclei were embedded in 5 ml of 0.8% (weight/volume [w/v]) low-melting-point agarose plugs and digested with Proteinase K. Forty plugs were stored in 40 ml of 50 mM ethylenediaminetetraacetic acid (EDTA) pH 8.0 and 0.4 g dithiothreitol at 4° C.

Fosmid Library Construction and Characterization. A fosmid library was constructed using the COPY CONTROL™ Fosmid Library Production Kit with pCC1FOS™ vector (LGC Biosearch Technologies). The protocol provided with the COPY CONTROL™ Fosmid Library Production K it was followed for all aspects of fragment end-repair, vector ligation, phage packaging, library titration, and unamplified library storage.

After fosmid library construction, the library was further characterized. Library sizing was performed on 13 randomly selected clones. Fosmid DNA was isolated using the QIAGEN Plasmid Maxi Kit (Qiagen Inc.) and was digested with a NotI restriction endonuclease (New England Biolabs) according to the manufacturer's protocol. Following size fractionation by PFGE (pulsed-field gel electrophoresis) the average insert size was calculated. Library sizing results indicated all 13 randomly-selected clones contained inserts and the average insert size was approximately 38 Kb. Genomic coverage was calculated based on an estimated maize genome size of 2.67 gigabase pairs (Gb) (Arumuganathan and Earle, 1991, Plant Mol Biol Rep 9:208-218) (Genomic coverage=Total number of clones x Average insert size/Estimated genome size). A total of 922,560 MZIR260 fosmid clones were obtained and consequently the genomic coverage for the library was estimated to be approximately 13×.

Fosmid Library Screening. A total of twenty-four pooled cell aliquots were obtained by collecting a fraction of cells from two 96-well plates. DNA was isolated using the QIAGEN Plasmid Maxi Kit (Qiagen Inc.) and was used as template in PCR analyses (Tables 7-10) to identify positive pools for Event MZIR260.

Following F positive pool identification, single clones were isolated by arraying recombinant clones originally found in the positive pool. Each positive pool was arrayed into 384-well plates followed by overnight incubation 37° C. DNA was isolated using the QIA GEN Plasmid Maxi Kit (Qiagen, Inc.) and PCR was performed (Tables 7-10) to further identify row pools positive for Event MZIR260. Once a positive row pool was identified, PCR analyses were performed using DNA from each of the 24 cells in the positive pool row. DNA from positive clones was isolated using the QIA GEN® Plasmid Maxi Kit (Qiagen, Inc.) and digested with the restriction enzyme NotI to further verify presence of insert.

TABLE 7
PCR Components for Event MZIR260 Fosmid Library Screening

| Reagent | Final concentration |
|---|---|
| 2X Bio S&T proprietary buffer | N/A |
| Primer forward | 0.33 μM |
| Primer reverse | 0.33 μM |
| ddH₂O | N/A |
| Template | N/A |
| RNase A (Qiagen, Inc.) | 0.7 units (0.03 mM) |
| Taq DNA Polymerase (Qiagen, Inc.) | 1.5 units Taq polymerase |
| Total | N/A |

N/A = not applicable.

TABLE 8
Primers Used for M ZIR 260 Fosmid
Library PCR Screening

| PCR | | Primer | SEQ |
|---|---|---|---|
| assay | | sequence | ID |
| target | Primer name | 5′ to 3′ | NO |
| 5′ Genome- | M ZIR260_5′_F1 | CGGCTCGGTGG | 28 |
| to-Insert | | TTGTATAAG | |
| Junction | M ZIR260_5′_R1 | CCGTTAGGCTA | 29 |
| | | GTGCCAGTG | |
| eCry1Gb. | M ZIR260_eCry1Gb. | ACAAGGTGTCC | 30 |
| 1lg-03 | 1lg-03_F1 | AACCTCGTC | |
| | MZIR260_eCry1Gb. | CTCAGCTCGTA | 31 |
| | 1lg-03_R1 | GCGGGTGTA | |
| 3′ Insert- | M ZIR260_3′_F1 | CGTGAGCCTTT | 32 |
| to-Genome | | AGCAACTAGC | |
| Junction | M ZIR 260_3′_R1 | GTGGTGAGGTT | 33 |
| | | CGAGGACAT | |

TABLE 9
PCR Conditions Used for MZIR260 Fosmid Library
Screening (5′ Genome-to-Insert A ssay)

| Cycle | Step | Temperature (° C.) | Time | | Number of cycles |
|---|---|---|---|---|---|
| A | 1 | 94 | 5 | min | 1 |
| B | 1 | 94 | 30 | sec | 33 |
| | 2 | 60 | 40 | sec | |
| | 3 | 72 | 45 | sec | |
| C | 1 | 72 | 5 | min | 1 |

| | | | | |
|---|---|---|---|---|
| D | 1 | 4 | Hold | N/A |

TABLE 10
PCR Conditions Used for MZIR260 Fosmid Library Screening
(eCry1Gb.1lg-03 and 3′ Insert-to-Genome Junction A ssays)

| Cycle | Step | Temperature (° C.) | Time | | Number of cycles |
|---|---|---|---|---|---|
| 1 | 1 | 94 | 5 | min | 1 |
| B | 1 | 94 | 30 | sec | 33 |
| | 2 | 60 | 30 | sec | |
| | 3 | 72 | 45 | sec | |
| C | 1 | 72 | 5 | min | 1 |

| | | | | |
|---|---|---|---|---|
| D | 1 | 4 | Hold | N/A |

Pacific Biosciences (PacBio) Sequencing of Event MZIR260. DNA extracted from two selected fosmid clones positive for MZIR260 was sequenced in a PACBIO® SEQUEL 1I® platform at DNA Link, Inc. (Gangseo-gu, Seoul, Republic of Korea) using PACBIO® Single Molecule Real-Time (SMRT®) sequencing technology. A total of 1000 ng of DNA from each fosmid clone was sheared with a g-TUBE (Covaris) and used for library preparation following the SMRTBELL® Prep Kit 3.0 protocol (Pacific Biosciences). The sequencing libraries obtained were analyzed on the FEMTO PULSE® (Agilent), which indicated an average library length of 10139 bp. Library DNA concentration was measured with the Qubit double stranded DNA High Sensitivity assay kit (Thermo Fisher Scientific). The cleaned, pooled libraries were sequenced using a single 8M SMRT® Cell on the SEQUEL II® System with SEQUEL II® Sequencing Kit, 30 hours movies, and SMRT® Link software v11.1. SMRT® Analysis software v11.1 was used to trim adapters and generate subreads.

Circular Consensus Sequencing (CCS) reads (CCS reads are consensus sequences resulting from alignment between subreads from a single polymerase read) were generated using SMRT® Link Analysis Software. Subsequently, PACBIO® CCS reads were analyzed using bioinformatic analysis.

CCS reads were analyzed to determine the MZIR260 insert and maize genomic flanking sequences. Sequencing results indicated that the average per-base coverage for the two fosmid clones was 106704X and 89206X. Variant analysis was conducted using bioinformatics analysis as well as by visual inspection using IGV (Integrated Genome Viewer). One of the putative variants identified in one of the fosmid clones was in a stretch of 13 cytosines homopolymer region located in the maize-derived ubiquitin promoter (Table 11). This is not unexpected, as PACBIO® sequencing is known to exhibit a bias for insertions and deletions (indels) in homopolymer sequences similar to other next generation sequencing platforms (Ross et al. 2013, Genome Biol 14: R51; Wenger et al. 2019, Nat Biotechnol. 37:1155-1162), albeit to a lesser extent (Foox et al. 2021, Nat Biotechnol 39:1129-1140). Consequently, the cytosine homopolymer region from each of the two fosmid clones was further verified with Sanger sequencing using a SeqStudio™ Genetic Analyzer and BIGDYE® v. 3.1 chemistry. The forward and reverse primers used to sequence each clone are listed in Table 11. The Sanger sequencing results, which supersede those from PACBIO® sequencing, match the reference sequence and indicated the presence of 13 cytosines in each of the fosmid clones.

TABLE 11
Primers Used for Sequencing M ZIR 260
Homopolymer Region

| | Primer sequence | SEQ ID |
|---|---|---|
| Primer name | (5′ to 3′) | NO |
| M ZIR_260 Ubi_F | CTTTCCCCAACCTCGTGTT | 34 |
| M ZIR 260_Ubi_R | AACGCTAACAGCACGGATCT | 35 |

Comparison of the MZIR260 insert and flanking consensus sequence (derived from PACBIO® and Sanger sequencing results) to the transformation plasmid 24795 showed that the MZIR260 insert is intact with no rearrangements, and only one base pair change within the sugarcane-derived ubiquitin promoter (from Guanine to Thymine at position 1786) was found in the MZIR260 insert and flanking sequence. Truncation was observed at the right and left border sequences of the 24795 T-DNA during the transformation process that resulted in MZIR260 maize. Since these truncations are located outside of the expression cassettes found in the MZIR260 insert, they do not affect the functionality of the elements contained therein.

### Example 5. Genomic Insertion Site of MZIR260

The genomic insertion site of the MZIR260 insert in the maize genome was determined by sequencing non-transgenic NP2222 maize, a near-isogenic line to MZIR260 maize. The NP2222 maize DNA sequence at the point of integration of the MZIR260 insert was first amplified by PCR using an Invitrogen Platinum™ SuperFI™ II Green PCR Master Mix. PCR was carried out using NP2222 genomic DNA as template and PCR primers (Table 12) in an Eppendorf MASTERCYCLER® X50s. Table 13 lists the thermal cycling parameters for PCR amplification.

TABLE 12
PCR Primers for Amplification of
the Genomic Insertion Site of the
M ZIR 260 Insert

| | Length | Primer sequence | SEQ ID |
|---|---|---|---|
| Primer name | (bp) | 5′ to 3′ | NO |
| NP2222_ | 21 | TCCCTTCGTTC | 36 |
| 260GIS_F3 | | CATGCCTAAA | |
| NP2222_ | 20 | ACTCAGTGGTA | 37 |
| 260GIS_R3 | | TTGGTCGCA | |

TABLE 13
Thermal Cycling Parameters for PCR Amplification

| Cycle | Step | Temperature (° C.) | Time | | Number of cycles |
|---|---|---|---|---|---|
| A | 1 | 98 | 30 | s | 1 |
| B | 1 | 98 | 10 | s | 35 |
| | 2 | 60 | 10 | s | |
| | 3 | 72 | 2 | min | |
| C | 1 | 72 | 5 | min | 1 |

| | | | | |
|---|---|---|---|---|
| D | 1 | 4 | hold | 1 |

The PCR reactions were done in triplicate and were individually run on an agarose gel, after which fragments of expected size were excised and purified using a QIAQUICK® Gel Extraction Kit according to the kit manufacturer's instructions. The three purified PCR reactions were cloned separately into the Invitrogen™ PCR-XL-2-TOPO™ vector and the cloning reactions were transformed into ONE SHOT™ OMNIMAX™ 2 T1® Chemically Competent Escherichia coli cells. From each cloning reaction, three colonies were randomly selected, and plasmid DNA was prepared using Promega PUREYIELD™ Plasmid MiniPrep System. Positive clones were confirmed by restriction enzyme digestion of plasmid DNA, and one clone from each of the three cloning reactions was randomly selected.

Each of the inserts in these three PCR clones were fully and independently sequenced using sequencing primers listed in Table 15. Sequencing was performed using Applied Biosystems BIGDYE® version 3.1 Cycle Sequencing Kit. The reactions were then run on an Applied Biosystems SeqStudio Genetic Analyzer. Resulting Sanger reads were manually trimmed to remove ambiguous ends using GENEIOUS PRIME® 2020.1.2. For each PCR clone, the trimmed reads were assembled de novo with Geneious assembler. The consensus sequences resulting from the read assembly of each PCR clone were aligned using multiple sequence alignment tool in GENEIOUS PRIME® 2020.1.2 to generate the final consensus sequence. Finally, the primer sequences (Table 14) used to amplify the genomic insertion site PCR fragment were trimmed from the final consensus sequence to obtain the genomic insertion site sequence for the NP2222 nontransgenic locus where MZIR260 T-DNA is inserted (SEQ ID NO: 77).

TABLE 14
Sequencing Primers for Genomic
Insertion Site of PCR Clones

| | | | SEQ |
|---|---|---|---|
| | Length | Primer sequence | ID |
| Primer name | (bp) | (5′ to 3′) | NO |
| NP2222_260GIS_F2 | 20 | TGGTGCCACAGTTGTATGCT | 38 |
| NP2222_260GIS_R2 | 20 | GTCATTGAGCAGACCCACCA | 39 |
| NP2222_260GIS-F4 | 21 | CGACTGCTAGCCAATCTTCCA | 40 |
| NP2222_260GIS-F5 | 20 | CGAGCACGGCAGTCATAGAT | 41 |
| NP2222_260GIS-R5 | 23 | CCGGTACTAATGACATTTGTG | 42 |
| | | CA | |
| NP2222_260GIS-F6 | 20 | TTCCAGCACGAGTGAAGCAT | 43 |
| NP2222_260GIS-R6 | 20 | TCAGGAAGATGGCGGGAGTA | 44 |
| NP2222_260GIS-F7 | 20 | ATCAGGCTTCTGTCCAGCAC | 45 |
| NP2222_260GIS-R7 | 21 | ACATCATCAGTTGGAGTGCGT | 46 |
| NP2222_260GIS-F8 | 21 | CGGTTGAAGACACCAAACAGT | 47 |
| NP2222_260GIS-R8 | 22 | TGTCCCAATGTCTCTAACACGG | 48 |
| NP2222_260GIS-F9 | 21 | TGGCAGAAGTTTTGGGTTCCT | 49 |
| NP2222_260GIS-R9 | 20 | GCAGCTTGCGTTGACGAATT | 50 |
| NP2222_260GIS- | 22 | AGTCTCAATTTCCCTTGGTCCC | 51 |
| R10 | | | |

This genomic insertion site sequence from nontransgenic NP2222 maize was then compared to the 5′ 1000 bp (SEQ ID NO: 8) and 3′ 1000 bp (SEQ ID NO: 9) sequences flanking the MZIR260 insert in MZIR260 maize. Sequence analysis of the MZIR260 genomic insertion site demonstrated that 30 bp region from the native maize genomic sequence was deleted when the MZIR260 insert integrated into the maize genome.

### Example 6. Chromosomal Location of MZIR260

The nucleotide sequence of NP2222 maize genomic site where the MZIR260 insert integrated was used to determine the chromosomal location of the MZIR260 insert.

Using the Basic Local Alignment Search Tool for Nucleotides (BLASTN) program, version 2.10.1+ (Zhang et al. 2000, J Comput Biol 7:203-14), the NP2222 maize genomic sequence where MZIR260 insert integrated was screened for similarity to DNA sequences in maize using the latest version of the B 73 maize reference genome (B 73 RefGen_v5) (on the worldwide web at maizegdb.org/assembly; Woodhouse et al., 2021, BMC Plant Biol 21: 1-10). This last version of the B 73 maize genome assembly was released in January 2020 (on the worldwide web at maizegdb.org/genome/assembly/Zm-B 73-REFERENCE-NAM-5.0).

The BLASTN results summarized in Table 15 showed that several regions of maize chromosome 2 share homology to the genomic insertion site sequence of MZIR260. The maize region with highest similarity (99%) and with an E-value=0 (i.e., a measure of the probability that matches between sequences occurred by chance) is located in chromosome 2 between bp 140849156 and bp 140851379 based on the publicly available B 73 maize genome.

TABLE 15
B73 Maize Chromosome Regions with Similarity
to MZIR260 Genomic Insertion Site

| Maize | Starting | Ending | Percent | E- |
|---|---|---|---|---|
| chromosome | base pair | base pair | identity | value |
| 2 | 140849156 | 140851379 | 99 | 0 |
| 2 | 141017217 | 141018496 | 88 | 0 |
| 2 | 141018952 | 141019701 | 89 | 0 |
| 2 | 141068524 | 141068636 | 97 | 3 × 10⁻⁴⁶ |
| 2 | 141304609 | 141304354 | 78 | 1 × 10⁻³⁶ |

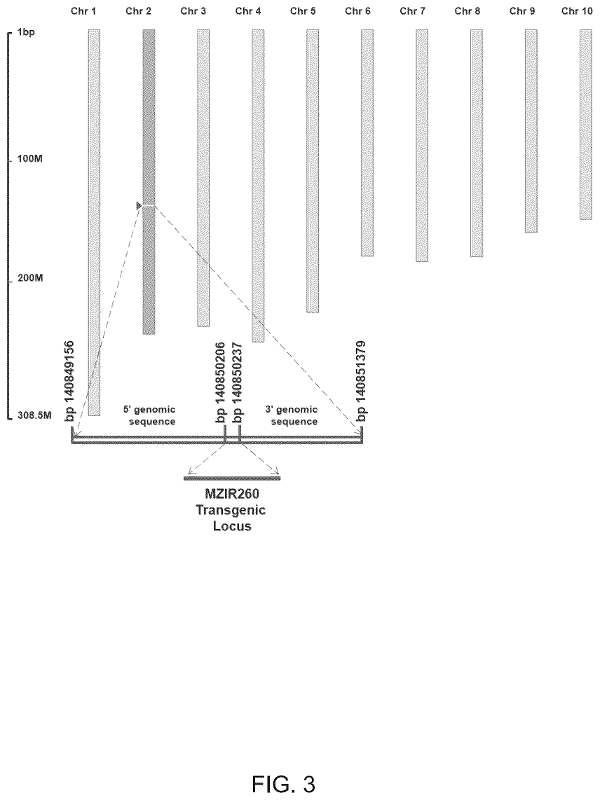

A schematic representation of the B 73 maize chromosome 2 region with highest similarity to MZIR260 genomic insertion site is shown in FIG. 3 and indicates the chromosomal location of the MZIR260 transgenic locus.

### Example 7. T-DNA Insert Sequencing

The nucleotide sequence of the entire transgene DNA insert present in event MZIR260 was determined to demonstrate overall integrity of the insert, contiguousness of the functional elements and to detect any individual base pair changes. The event MZIR260 insert was PCR amplified from genomic DNA derived from individual plants as overlapping fragments to cover the entire T-DNA insert which is linked to its 5′ flanking sequence and 3′ flanking sequence on each side.

TABLE 16
Primer sequence combinations
used for overlapping PCR

| PCR | SEQ ID | |
|---|---|---|
| Amplicon | NO. | Sequence (5′ to 3′) |
| Amplicon-A | 52 | TCCTCCCGTCTCTCGATTCC |
| | 53 | CGTTCAGGATCTCGGACTCG |
| Amplicon-B | 54 | TGCTGTATGTGCCTTCTGCT |
| | 55 | GTCGTACTGGCTGTTGACGA |
| Amplicon-C | 56 | TTACGTCGCGGAGAGATGGAT |
| | 57 | GTGTCCGTTAGACTCGTCGG |
| Amplicon-D | 58 | TTGCAATCCCATACTAATTAG |
| | | CTAACGG |
| | 59 | GCGAGAAGGCAAAATCGTCC |
| Amplicon-E | 60 | CGCCAGCCTGTTGAATATGC |
| | 37 | ACTCAGTGGTATTGGTCGCA |

PCR amplification was carried out using a high fidelity enzyme (Q5® Hot Start High-Fidelity 2X Master Mix, New England BioLabs (NEB), M 0494L) with PCR parameters adjusted for different target regions. In one example, PCR was carried out using the following parameters: 30 sec at 98° C. for 1 cycle, followed by 35 cycles of 10s at 98° C., 30s at 68° C. and 1.5 min at 72° C., followed by 1 cycle of 2 min at 72° C. PCR product obtained from the overlapping PCR amplification was purified with Ampure XP bead-based reagent (Beckman coulter, A 63881) according to the manufacturer's protocol.

Sequencing was carried out using Illumina MiSeq 250 PE with Nextra XT v2 index Kit D (raw data was generated by M O gene). The final consensus sequence was determined by combining the sequence data from different PCR amplicons to generate consensus sequence of the event MZIR260 insert (SEQ ID NO: 5) linked to its 5′ genome sequence and 3′ genome sequence at each side (SEQ ID NO: 8 and SEQ ID NO: 9). SEQ ID NO: 6 comprises the full-length T-DNA insertion with 5′ and 3′ flanking genomic sequences. The consensus sequence data for the event MZIR260 insert demonstrates that the overall integrity of the insert and contiguousness of the functional elements within the insert as intended in 24795 have been maintained. The nucleotide sequence analysis thus demonstrated that the MZIR260 insert contains a single copy of each of the functional elements (Ubi4-02 promoter, eCry1Gb.1Ig-03, Ubi361-05 terminator, Ubi1-43 promoter, pmi-15, and Ubi1-04 terminator).

The homopolymer region in Ubi1-43 promoter was indeterminable when using the PCR amplicon as sequence material. Therefore, PCR-free CRISPR-CATCH (Cas12a assisted targeting of chromosome segment) subcloning method followed by Sanger sequencing was used for homopolymer region analysis of MZIR260. Two CRISPR-RNAs (crRNAs) that specifically target 5′ and 3′ flanked regions of interest were used for digesting and releasing the region of interest from genomic DNA of MZIR260. In vitro ribonucleoprotein (RNP) assay was performed using 10 μg of genomic DNA of MZIR260, 6 μg of EnGen Lba Cas12a (Cpf1) (NEB, M 0653T), and 2.1 ug of crRNAs. The digested genomic fragment was isolated through agarose gel electrophoresis, purified with column base nucleotide purification kit (QIA quick Gel Extraction Kit, QIAGEN, 28704), and subcloned into pre-designed cloning vector, which was amplified using pU C19 vector as template, using seamless cloning reagent (NEBuilder HiFi DNA assembly Master Mix (NEB, E2621L). The assembly mixture was transformed into E. coli competent cells (ONE SHOT® TOP10 ELECTROCOMP™ E. coli cells, Invitrogen, C4040-52), and positive colonies were selected by colony PCR. The homopolymer region of positive clones were sequenced by Sanger sequencing (Genewiz). With this method, the homopolymer region of MZIR260 was determined (Table 17). The primer sequences used for PCR are listed in Table 18.

TABLE 17
crRNA sequence of CRISPR-CATCH

| CRISPR- | SEQ | |
|---|---|---|
| CATCH | ID | |
| fragment | NO. | crRNA sequence |
| Fragment- | 61 | /AITR1/rUrArArUrUrUrCrUrAr |
| A | | CrUrArArGrUrGrUrArGrArUrAr |
| | | CrArGrGrCrUrGrGrCrArUrUrAr/ |
| | | AITR2/ |
| | | UrCrUrArCrUrCrGrArA |
| | 62 | /AITR1/rUrArArUrUrUrCrUrAr |
| | | CrUrArArGrUrGrUrArGrArUrCr |
| | | ArUrArCrCrGrUrArArArGrUrUr |
| | | CrArGrUrCrArArCrGrC/AITR2/ |

TABLE 18
Primer sequence used for CRISPR-CATCH

| CRISPR- | SEQ | |
|---|---|---|
| CATCH | ID | |
| vector | NO. | Sequence (5′ to 3′) |
| Subcloning | 63 | AGTGCAAAACTATGCCTGGGGCAG |
| vector | | CAAAACTCGAATTCACTGGCCGTC |
| amplifi- | 64 | CGGCGTTTAACAGGCTGGCATTAT |
| cation | | CTACTCATGCAAGCTTGGCGTAATC |
| Colony | 65 | AGCGGATAACAATTTCACACAGG |
| PCR-1 | 66 | GACCCGACAAACAAGTGCAC |
| Colony | 67 | TCTAACGGACACCAACCAGC |
| PCR-2 | 68 | CCCAGTCACGACGTTGTAAAACG |
| Primer for | 67 | TCTAACGGACACCAACCAGC |
| Sanger | | |
| sequencing | 66 | GACCCGACAAACAAGTGCAC |

### Example 8. Detection of eCry1Gb.1Ig Via ELISA

The concentrations of eCry1Gb.1Ig protein in leaf and kernel tissues of MZIR260 hybrid plants were quantified by enzyme-linked immunosorbent assay (ELISA). The MZIR260 hybrid and the corresponding nontransgenic, near-isogenic maize hybrid were grown at four filed trial locations in the United States in 2022. The genotypes of the plants used in these studies were IJ 7010/N P 2222 (MZIR260) and IJ 7010/N P 2222.

At each location, one plot was planted with MZIR260 maize, and one plot was planted with nontransgenic, near-isogenic maize. Five replicate samples of each tissue type were collected from each plot for MZIR260; two replicate samples were collected for the nontransgenic control plot. For leaves, all the true leaves from five plants were pooled per sample. For kernels, all the kernels from the primary ears from five plant were pooled per sample. All tissue samples were ground to a powder, and all samples were then lyophilized. The percent dry weight (DW) of each sample was determined from the sample fresh weight before, and the sample dry weight after, lyophilization.

Protein was extracted from representative aliquots of the lyophilized tissue samples at a ratio of 3 ml protein extraction buffer (50 mM ncyclohexyl-3-aminopropanesulfonic acid (CAPS), pH 10.5, 0.2% bovine serum albumin (BSA), 0.1% Tween® 20, 1 mM tris(2-carboxyethyl) phosphine (TCEP)) to approximately 30 mg of lyophilized tissue. The samples were homogenized, centrifuged, and the supernatant was collected. Samples were diluted in ELISA diluent (PBS, pH 7.4 containing 0.05% Tween-20).

The eCry1Gb.1Ig protein was quantified using a sandwich ELISA with a pair of monoclonal antibodies specific to the eCry1Gb.1Ig protein, following standard techniques. The sample extracts were analyzed for each trait protein, and a standard curve was generated for each ELISA plate with known amounts of the corresponding reference protein. Concurrent analysis of tissues from the nontransgenic corn confirmed the absence of plant-matrix effects on the analysis methods. All protein concentrations were adjusted for extraction efficiency (Table 19).

TABLE 19
Concentration of eCry1Gb.1lg protein in V6 leaves
and R6 kernels of MZIR260 maize plants

| | | Mean ± SD |
|---|---|---|
| Stage^{a, b} | Tissue | (μg/g FW^{c}) |
| V6 (BBCH 16) | Leaves | 35.5 ± 12.2 |
| R6 (BBCH 87) | Kernels | 195 ± 25.0 |

^{a}V-R scale (Abendroth et al. 2011, PMR 1009. Ames, IA: Iowa State University Extension. 49 pp).
^{b}BBCH = Biologische Bundesanstalt, Bundessortenamt and Chemische Industrie scale (Meier 2001, BBCH Monograph. 2nd ed. Braunschweig, Germany: German Federal Biological Research Centre for Agriculture and Forestry. 158 pp.
^{c}FW = fresh weight

### Example 9. Insect Control Field Trial Data

Spodoptera frugiperda

In-field Spodoptera frugiperda, fall armyworm (FAW) efficacy experiments were performed to test the top four different transgenic events from construct 24795 (MZIR260 and Events A, B, and C). Main season (Safra) and second season (Safrinha) are presented.

In main season, trials across four locations and three replicates per location were analyzed. Events were evaluated within four testers. In second season, trials across three locations, three replicates in each location, and three testers were analyzed.

There were three replicates per location. In each replicate plot there were four rows of 80 plants total, and all four rows were infested with 20 FAW neonates per plant. Ratings the inner two rows were evaluated for FAW damage. The Davis Scale was used for evaluating damage where a 0 represented no observed damage and 9 indicated complete destruction of the whorl and plant.

In each season, similar infestation and rating methods were utilized Davis ratings were log transformed and analyzed with mixed model analysis of variance in SAS v9.4 (SAS Institute, Cary, NC) to test the main effect of event accounting for the random effects of location and rep within location. Tukey's adjustment was used for post-A NOVA comparisons. Untransformed least square means and standard errors are presented below.

Results from those FAW trials are presented below (Tables 20 and 21).

TABLE 20
FAW Main Season: Assessed in 4 locations across 4 testers.

| | FAW Davis | Std |
|---|---|---|
| Event | LS Mean | err |
| MZIR260 | 1.67 | 0.21 |
| Event A | 1.58 | 0.21 |
| Event B | 1.69 | 0.21 |
| Event C | 1.95 | 0.21 |
| Non-GM | 5.09 | 0.21 |

TABLE 21
FAW Second Season: Assessed in 3 locations across 3 testers

| | FAW Davis | Std |
|---|---|---|
| Event | LS Mean | err |
| MZIR260 | 1.24 | 0.36 |
| Event A | 1.17 | 0.36 |
| Event B | 1.11 | 0.36 |
| Event C | 1.57 | 0.36 |
| Non-GM | 4.70 | 0.35 |

Diatraea saccharalis

A sugarcane borer (SCB) trial was performed in Argentina in one season testing 4 transgenic events from construct 24795. At each of four locations, three replicate plots per event were infested and 40 plants per replicate were observed for SCB holes and tunneling.

Total SCB holes were evaluated and due to the limited number of holes observed, the binary outcome of absence or presence of SCB holes was used for statistical comparisons between events. Generalized linear mixed model was used to test if there were differences in the probability for events to have SCB holes. Model-adjusted probabilities and standard errors are presented in Table 22.

TABLE 22
SCB:Assessed in 4 locations across 3 testers

| | Model-adj. probability | Std |
|---|---|---|
| Event | SCB Holes | err |
| MZIR260 | 0.007 | 0.002 |
| Event A | 0.014 | 0.003 |
| Event B | 0.005 | 0.002 |
| Event C | 0.004 | 0.004 |
| Non-GM | 0.437 | 0.01 |

Spodoptera litura and Spodoptera frugiperda Efficacy in China

In-field and in-greenhouse Spodoptera litura, Common Cutworm (CCW) efficacy experiments were performed to test MZIR260. Data from one greenhouse study and one field trial are presented.

In the greenhouse study, all entries were arranged in a randomized complete block design with three replicates. In each replicate there were 20 plants total, each plant was grown in an individual pot, and all plants were artificially infested with 20 to 30 CCW neonates per plant at V 4 to V 6 maize growth stage. Ratings were evaluated for CCW damage. The Davis Scale was used for evaluating damage where a 0 represented no observed damage and 9 indicated complete destruction of the whorl and plant.

The Davis ratings were analyzed with analysis of variance in SAS v9.2 (SAS Institute, Cary, NC). Least Significant Difference (LSD) test was used for post-ANOVA comparisons. Means and standard deviations are presented below (Table 23).

TABLE 23
CCW Greenhouse Study

| | CCW damage rating |
|---|---|
| Event | (mean ± standard deviation) |
| MZIR260 | 0.9 ± 0.1 |
| Non-GM | 8.0 ± 0.0 |

In the field trial, all entries were arranged in a randomized complete block design with three replicates. Two inner rows of 42 plants total were infested with 30 to 40 CCW neonates per plant at V 4 to V 6 maize growth stage. Damage rating scale specified in China Ministry of Agriculture and Rural Affairs (MARA) notice 953-10.1-2007 (see Table 24) was used for evaluating damage where a value of 1 represented no observed damage or only small (≤1 mm) pinhole damage, and a value of 9 indicated complete destruction of the whorl and plant.

TABLE 24
Leaf Damage Scale

| Leaf damage | Description/ |
|---|---|
| rating | Symptoms |
| 1 | No damage, or only pin size holes (≤1 mm) observed in the leaves |
| 2 | A few bullet sized (≤5 mm) holes on few individual leaves |
| 3 | Bullet sized (≤5 mm) pinholes observed on several leaves |
| 4 | Ragged edge cut (≤10 mm) observed on individual leaves |
| 5 | Ragged edge cut (≤10 mm) observed on a few of leaves |
| 6 | Ragged edge cut (≤10 mm) observed on some leaves |
| 7 | Individual leaves are partially eaten, large area of ragged edge cut (≤10 mm) |
| | observed on few leaves |
| 8 | Few leaves are eaten, large area of ragged edge cut (≤10 mm) observed on |
| | some leaves |
| 9 | Large area or complete destruction of leaves and plant |

The damage ratings were analyzed with analysis of variance in Data Processing System (DPS). Duncan's Multiple Range Test (MRT) was used for post-A NOVA comparisons. Means and standard deviations are presented below (Table 25).

TABLE 25
CCW field trial in CN

| | CCW damage rating |
|---|---|
| Event | (mean ± standard deviation) |
| MZIR260 | 1.18 ± 0.11 |
| Non-GM | 8.14 ± 0.76 |
| Non-GM reference hybrid | 8.33 ± 0.25 |

In-field Spodoptera frugiperda, fall armyworm (FAW) efficacy experiments were performed to test the top transgenic event from construct 24795 (MZIR260). Trials across four locations and two years are presented.

There were three replicates per location. In each replicate plot, 20 consecutive plants from two rows were evaluated for FAW damage under natural infestation. The Davis Scale was used for evaluating damage where a 0 represented no observed damage and 9 indicated complete destruction of the whorl and plant.

The damage ratings were analyzed with analysis of variance in Data Processing System (DPS). Duncan's Multiple Range Test (MRT) was used for post-A NOVA comparisons. Means and standard deviations from each individual field trial are presented below (Tables 26-29).

TABLE 26
FAW field trial in CN Location 1 (Year 1)

| | FAW damage rating |
|---|---|
| Event | (mean ± standard deviation) |
| MZIR260 | 1.02 ± 0.03 |
| Non-GM | 5.78 ± 1.86 |
| Non-GM reference hybrid | 4.92 ± 0.54 |

TABLE 27
FAW field trial in CN Location 2 (Year 1)

| | FAW damage rating |
|---|---|
| Event | (mean ± standard deviation) |
| MZIR260 | 1.10 ± 0.05 |
| Non-GM | 5.70 ± 0.26 |
| Non-GM reference hybrid | 4.87 ± 0.53 |

TABLE 28
FAW field trial in CN Location 1 (Year 2)

| | FAW damage rating |
|---|---|
| Event | (mean ± standard deviation) |
| MZIR260 | 1.08 ± 0.08 |
| Non-GM | 6.37 ± 0.25 |
| Non-GM reference hybrid | 6.67 ± 0.33 |

TABLE 29
FAW field trial in CN Location 2 (Year 2)

| | FAW damage rating |
|---|---|
| Event | (mean ± standard deviation) |
| MZIR260 | 1.01 ± 0.01 |
| Non-GM | 8.89 ± 0.05 |
| Non-GM reference hybrid | 8.86 ± 0.06 |

### Example 10. Agronomy Field Trial Data for MZIR260 vs. Other Events with Same Construct

Field trials were performed to compare agronomic performance of four transgenic events from construct 24795 in Argentina, the US, and Brazil. A near isogenic non-traited (non-GM) control was included as a comparator in all trials.

In each of the Argentina and US seasons, six locations were utilized with three replicates per location and events were compared across four testers for agronomic performance. In the Brazil season, three locations were utilized with three replicates per location and events were compared across four testers for agronomic performance.

In each season, yield (bu/ac) was analyzed with mixed model analysis of variance in SAS v9.4 (SAS Institute, Cary, NC) to test the main effect of event accounting for the random effects of location and rep within location. Tukey's adjustment was used for post-A NOVA comparisons. Least square means and standard errors are presented below. The results are in Tables 30-32. Based on the data in these Examples, only event MZIR260 had acceptable efficacy and agronomic performance across multiple testers and locations, and had acceptable insertion site characteristics to make it an elite event.

TABLE 30
Yield Results in Brazil

| Brazil | LS mean Yield | Std |
|---|---|---|
| (3 locs, 4 testers) | (bu/ac) | Err |
| MZIR260 | 95.2 | 29.6 |
| Event A | 92.5 | 29.6 |
| Event B | 97.3 | 29.6 |
| Event C | 92.9 | 29.6 |
| Non-GM | 97.0 | 29.6 |

TABLE 31
Yield Results in the US

| USA | LS mean Yield | Std |
|---|---|---|
| (6 locs, 4 testers) | (bu/ac) | Err |
| MZIR260 | 164.0 | 6.9 |
| Event A | 152.8 | 6.9 |
| Event B | 156.0 | 6.9 |
| Event C | 158.2 | 6.9 |
| Non-GM | 165.6 | 6.9 |

TABLE 32
Yield Results in Argentina

| Argentina | LS mean Yield | Std |
|---|---|---|
| (6 locs, 4 testers) | (bu/ac) | Err |
| MZIR260 | 155.8 | 21.2 |
| Event A | 154.4 | 21.2 |
| Event B | 159.1 | 21.2 |
| Event C | 161.7 | 21.2 |
| Non-GM | 158.8 | 21.2 |

### Example 11: Modification of Maize Event MZIR260 by Genome Editing Using a Single gRNA

This example describes how one may alter or excise all or a part of the transgenic insertion present in maize event MZIR260, as well as flanking genomic DNA segments, such as by making one or more insertions, deletions, substitutions, or transpositions using genomic editing techniques. For example, such alterations can be made using Clustered Regularly Interspersed Short Palindromic Repeats (CRISPR) editing systems comprising a single guide RNA by genome editing methods. Sequences useful in excision of the event MZIR260 transgenic insertion or expression cassettes within SEQ ID NO: 5 can be introduced through genome editing using a variety of methods. In one embodiment, Clustered Regularly Interspersed Short Palindromic Repeats (CRISPR) editing systems comprising a CRISPR associated protein and cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CsbI, Csb2, Csb3, Csx17, Csx14, Csx10, Csx16, CsaX, Csx3, Csx1, Csx15, CsfI, Csf2, Csf3, Csf4, CasX, CasY, and Mad7. The CRISPR-associated protein and one or more guide RNAs (gRNA) can be introduced into a plant cell corresponding to maize event MZIR260 to target a specific sequence within the transgene insertion locus. In one embodiment, the CRISPR nuclease system cleaves at two identical guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis dependent strand annealing (SDSA), single-strand annealing (SSA), or a combination of any thereof, at the genomic target site. One or more guide RNA hybridization sequences can be inserted within the event MZIR260 transgene insertion locus which can subsequently allow for the excision of the transgene insertion from event MZIR260 or specific expression cassettes within SEQ ID NO: 5.

Sequences corresponding to the 5′ and 3′ flanking genomic sequences of event MZIR260 (presented as SEQ ID NOs: 8 and 9), the 5′ and 3′ junction regions (presented as SEQ ID NOs: 1-4) and the transgenic insertion (presented as SEQ ID NO: 5) are scanned for potential originator guide RNA recognition sites (OgRRS). As used herein, the term “originator guide RNA recognition site” or “OgRRS” refers to an endogenous DNA polynucleotide comprising a protospacer adjacent motif (PAM) site operably linked to a guide RNA hybridization site (i.e., protospacer sequence). In some embodiments, an OgRRS can be located in the flanking 5′ or 3′ genomic sequence (i.e., in non-transgenic DNA of a junction polynucleotide). In some embodiments, an OgRRS can be located in the 5′ or 3′ junction region (i.e., in both transgenic DNA and non-transgenic DNA of a junction polynucleotide, or spanning transgenic and nontransgenic DNA in a DNA junction polynucleotide). In some embodiments, an OgRRS can be located in the transgenic insert. The OgRRS can be determined based upon the specific CRISPR editing system chosen. For example, Cas9 recognizes a G-rich protospacer-adjacent motif (PA M) that is 3′ to its guide RNA hybridization site whereas CasI2a systems recognize a T-rich protospacer-adjacent motif (PAM) that is 5′ to its guide RNA hybridization site. Commercially-available programs are available for identifying PAM s within a target sequence including, but not limited to, Geneious Prime (Boston, MA), SnapGene (Boston MA), Integrated DNA Technologies, Inc. (Coralville, IA), and Spacer2PAM (available on the worldwide web at bio.tools/spacer2pam).

The OgRRS sequence is then used to define a cognate guide RNA recognition site (CgRRS) which is inserted into the transgenic insertion locus of event MZIR260 using a CRISPR editing system. As used herein, the term “cognate guide RNA recognition site” or “CgRRS' refers to a DNA polynucleotide comprising a PAM site operably linked to a guide RNA hybridization site (i.e., protospacer sequence), where the CgRRS is absent from event MZIR260 comprising the original transgenic locus that is unmodified and where the CgRRS and its corresponding OgRRS can hybridize to a single gRNA. A CgRRS can be located in the flanking 5′ or 3′ genomic sequence (i.e., in non-transgenic DNA of a junction polynucleotide), in the 5′ or 3′ junction region (i.e., in both transgenic DNA and non-transgenic DNA of a junction polynucleotide, or spanning transgenic and non-transgenic DNA in a DNA junction polynucleotide), or in the transgenic insert. A CgRRS comprises the same gRNA target sequence as the corresponding OgRRS. The CgRRS is inserted in a region within the transgenic insertion locus of event MZIR260 that is on the opposite side of the transgenic insertion, relative to the OgRRS, in a manner that will permit the excision of a fragment of DNA corresponding to either the entire transgenic insertion of event MZIR260, or a fragment within the transgene insert of event MZIR260 such as an expression cassette or genetic element within the transgene cassette, using a single gRNA. For example, if the OgRRS is located within the 3′ flanking genomic sequence or the 3′ junction region, then the CgRRS can be inserted within the 5′ flanking genomic sequence, or the 5′ junction region, or within the transgene insert such as between expression cassettes or genetic elements within an expression cassette. Insertion of the CgRRS on the opposite side of the transgenic insertion or within the region between expression cassettes, relative to the OgRRS, allows for excision of the transgenic insertion or specific expression cassette(s) to be excised using a single gRNA. An OgRRS located between the expression cassettes of event MZIR260 can be used to design a CgRRS that can be inserted in either the 5′ or 3′-flanking genomic sequence to permit excision of one or more expression cassettes using a single gRNA. Once excised, the transgenic insert of MZIR260 can be introduced into another corn event using targeted insertion. After selection of a transgenic event comprising the introduced MZIR260 insert, the event can be bred into another germplasm.

The CORRS can be introduced into the transgenic insertion locus through multiple methods using a CRISPR system. For example, a CRISPR system can be utilized for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, resulting in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′ flanking sequence at the cut site of the protospacer in the genomic target site.

The CRISPR system can be introduced into event MZIR260 by several methods. One or more expression cassettes encoding the gRNA and/or CRISPR associated protein components of a Type I, Type II, Type III, Type IV, Type V, or Type VI CRISPR-Cas system is transiently introduced into a cell. The introduced one or more expression cassettes encoding the gRNA and/or CRISPR-associated protein, along with a DNA fragment comprising the CgRRS is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell. Double-stranded DNA fragments can also be transiently introduced into a cell along with one or more expression cassettes encoding the gRNA and/or CRISPR associated protein. The introduced double-stranded DNA fragments are provided in sufficient quantity to modify the cell but do not persist after a contemplated period of time has passed or after one or more cell divisions.

Alternatively, an expression construct comprising one or more expression cassettes for the expression of one or more gRNAs, and an expression construct encoding a Type I, Type II, Type III, Type IV, Type V, or Type VI CRISPR associated protein is stably transformed into event MZIR260 to modify the plant cell in the targeted region of the transgene insertion locus, to introduce the CgRRS within the desired target locus.

### Example 12. Modification of Maize E Vent MZIR260 by Genome Editing Using Two gRNAs

This example describes how one may excise the transgenic insertion present in maize event MZIR260 using CRISPR editing systems comprising two guide RNAs by genome editing methods. Excision of the event MZIR260 transgenic insertion or expression cassettes within SEQ ID NO: 5 can be performed through genome editing using a variety of methods. In one embodiment, Clustered Regularly Interspersed Short Palindromic Repeats (CRISPR) editing systems comprising a CRISPR associated protein and two cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a

Type IV CRISPR-associated protein, Type V CRISPR-associated protein, or a Type VI CRISPR-associated protein, such as but not limited to, CasI, CasIB, Cas2, Cas3, Cas4, Cas5, Cas6, Cas7, Cas8, Cas9 (also known as Csn1 and Csx12), Cas10, Cas 12a (also known as Cpf1), Cas12b, Cas13a, Cas14, CasX CsyI, Csy2, Csy3, Cse1, Cse2, CscI, Csc2, Csa5, Csn2, Csm2, Csm3, Csm4, Csm5, Csm6, CmrI Cmr3, Cmr4, Cmr5, Cmr6, CsbI, Csb2, Csb3, Csx17, CsxI4, CsxIO, Csx16, CsaX, Csx3, Csx1, Csx15, Csf1, Csf2, Csf3, Csf4, CasX, CasY, and Mad7. The CRISPR-associated protein and two guideRNAs (gRNA) can be introduced into a plant cell comprising the maize event MZIR260 to target a specific sequence within the transgene insertion locus. In one embodiment, the CRISPR nuclease system cleaves at two distinct guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site.

Sequences corresponding to the 5′ and 3′ flanking genomic sequences, and the transgenic insert of event MZIR260 (presented as SEQ ID NOs: 8, 9, and 5, respectively) and the 5′ and 3′ junction regions (presented as SEQ ID NOs: 1-4) are scanned for potential guide RNA recognition sites which comprise a protospacer adjacent motif (PAM) site that will be recognized by an RNA-guided (e.g., CRISPR-associated protein) endonuclease, operably linked to a guide RNA hybridization site. The identified gRNA recognition sites are located within the 5′ or 3′ flanking genomic sequence, within the 5′ or 3′ junction regions, or within the transgenic insertion.

Two functional guide RNAs (gRNAs) for an RNA guided nuclease system are created to target the event MZIR260 transgenic insertion locus in a manner that will permit the excision of a fragment of DNA corresponding to either the entire transgenic insertion of event MZIR260, or a fragment within the transgenic insertion of event MZIR260 such as an expression cassette or genetic element within the transgene cassette.

To excise the entire transgenic insertion of event MZIR260, a first gRNA targets an area in the 5′ flanking genomic sequence, and the second gRNA targets a region in the 3′ flanking genomic sequence. A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding an RNA guided nuclease. A second expression cassette comprises a promoter that is operable in a plant cell operably linked to a selectable marker gene. The construct also comprises expression cassettes comprising Polymerase III promoters operable in a plant cell operably linked to polynucleotides encoding the two gRNAs.

To facilitate excision of a fragment within the transgenic insertion of event MZIR260 such as an expression cassette near the 3′ flanking genomic sequence, the first gRNA targets an area in the 3′ flanking genomic sequence and the second gRNA targets a region in the 5′ flanking region genomic sequence. A T-DNA construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding an RNA guided nuclease. A second expression cassette comprises a promoter that is operable in a plant cell operably linked to a selectable marker gene. The construct also comprises expression cassettes comprising Polymerase III promoters operable in a plant cell operably linked to polynucleotides encoding the two gRNAs.

Following Agrobacterium-mediated transformation of maize event MZIR260, and upon expression of the integrated polynucleotides, the gRNAs guide the nuclease to each of the two target sites at the transgenic insertion locus, where the nuclease creates a double-stranded break at each target site, resulting in deletion of the region between the target sites, and non-homologous end-joining repair mechanisms joins the flanking regions. Suitable methods known in the art (e.g., PCR, DNA hybridization (Southern) blots, sequencing) are used to identify plants comprising a complete deletion.

### DEPOSIT

Applicants have made a deposit of corn seed of elite event MZIR260 disclosed above on Oct. 27, 2023 in accordance with the Budapest Treaty at the Bigelow Laboratory for Ocean Sciences, National Center for Marine Algae and Microbiota (NCMA), 60 Bigelow Drive, East Boothbay, Me. 04544 under NCMA Accession No. 202310010. The seed was tested on Nov. 3, 2023 and found to be viable. The deposit will be maintained in the depositary for a period of 30 years, or 5 years after the last request, or the effective life of the patent, whichever is longer, and will be replaced as necessary during that period. Applicants impose no restrictions on the availability of the deposited material from the NCMA; however, applicants have no authority to waive any restrictions imposed by law on the transfer of biological material or its transportation in commerce.

All publications and published patent documents cited in this specification are incorporated herein by reference to the same extent as if each individual publication or patent document was specifically and individually indicated to be incorporated by reference.

What is claimed is:

1. A transgenic corn plant or a seed thereof comprising elite event MZIR260, an example of said seed deposited as NCMA Accession No. 202310010.

2. The transgenic corn plant or seed of claim 1, which comprises SEQ ID NO: 1, SEQ ID NO: 2, SEQ ID NO: 3, SEQ ID NO: 4, SEQ ID NO: 5, or SEQ ID NO: 6.

3. A nucleic acid molecule containing a nucleic acid sequence comprising SEQ ID NO: 1, SEQ ID NO: 2, SEQ ID NO: 3, SEQ ID NO: 4, SEQ ID NO: 5 or SEQ ID NO: 6.

4. An amplicon comprising the nucleic acid molecule of claim 3.

5. A pair of polynucleotide primers comprising a first polynucleotide primer and a second polynucleotide primer which function together in the presence of a corn elite event MZIR260 DNA template in a sample to produce an amplicon diagnostic for the corn elite event MZIR260.

6. The pair of polynucleotide primers according to claim 5, wherein the corn elite event MZIR260 DNA template comprises SEQ ID NO:1, SEQ ID NO: 2, SEQ ID NO: 3, SEQ ID NO: 4, SEQ ID NO: 5 or SEQ ID NO: 6, or a complement thereof.

7. The pair of polynucleotide primers according to claim 5, wherein the first polynucleotide primer comprises at least 10 contiguous nucleotides as set forth in SEQ ID NO: 8, or the complements thereof.

8. The pair of polynucleotide primer according to claim 5, wherein the second polynucleotide primer comprises at least 10 contiguous nucleotides as set forth in SEQ ID NO: 9, or the complements thereof.

9. A method of detecting the presence of a nucleic acid molecule that is unique to elite event MZIR260 in a sample comprising corn nucleic acids, the method comprising:
a) providing a genomic DNA sample from a corn plant that comprises a nucleic acid molecule that is unique to elite event MZIR260;
b) combining the nucleic acid molecule with a pair of polynucleotide primers according to claim 5;
c) performing a nucleic acid amplification reaction which results in an amplicon diagnostic for the corn elite event MZIR260, wherein the amplicon comprises a nucleic acid sequence comprising SEQ ID NO: 1, SEQ ID NO: 2, SEQ ID NO: 3, SEQ ID NO: 4, SEQ ID NO: 5 or SEQ ID NO: 6, or a complement thereof; and
d) detecting the amplicon.

10. The method of claim 9, wherein the detecting of the amplicon comprises contacting the amplicon with a probe that is complementary to the amplicon.

11. The method of claim 9, wherein the detecting of the amplicon comprises sequencing the amplicon.

12. The method of claim 9, further comprising selecting a plant comprising the amplicon for the purposes of breeding corn comprising corn elite event MZIR260.

13. A DNA molecule comprising the amplicon produced by the method of claim 9.

14. A biological sample or an extract thereof derived from the transgenic corn plant of claim 1,
wherein the sample comprises a nucleotide sequence which comprises or is complementary to any one of SEQ ID NOs: 1-6.

15. A method of producing hybrid corn seed comprising corn elite event MZIR260, comprising:
a) crossing the transgenic corn plant of claim 2 with a second corn plant that is different from the transgenic corn plant of claim 2 to produce hybrid corn seed; and
b) harvesting the hybrid corn seeds produced.

16. A kit for detecting the presence of corn elite event MZIR260 nucleic acids in a biological sample, the kit comprising the pair of polynucleotide primers of claim 5.


SEQUENCE LISTING (79 sequences)

SEQ ID NO: 1 - DNA, 300 bp, synthetic construct
  source 1..300: mol_type = other DNA
ctcggtggttgtataagagcaatcctccgtgttagagacattgggacaatatagtgacattacgaagaaactgactagtaatgcaacagaattacaagagtaacgtatgatgtttgttaattggaatttgactagcaaataattaattgtaaggatatcagcaacccgagcacggcagtcatagatgatgggtgattcaaaaggtgtcactggcactagcctaacggtgttgactaactaggccgcttccctaattagctaacccgggggcgcgccgggacccgaattcattatgtggtc

SEQ ID NO: 2 - DNA, 351 bp, synthetic construct
  source 1..351: mol_type = other DNA
cctgcaggcccgggggcgcgccctaattagctaacggccaggatcgccgcgtgagcctttagcaactagctagattaattaacgcaatctgttattaagttgtctaagcgtcaatttgtttacaccacaatataaaaagtattgtttaattacataagatgtcaagtacactatggcgcctgggttatatttgtgtcaatgtaaatgtttatgagcatttcagtcacacagatcaattattaactagatcttttctacgcactccaactgatgatgtcctcgaacctcaccacagcgtgctacagtaggatagcctcagctcccctatgacagcttcacgaagaatcgttg

SEQ ID NO: 3 - DNA, 572 bp, synthetic construct
  source 1..572: mol_type = other DNA
caacgcaagctgcataccatcttgtatgatgttctgtagagttgggaaggaaatgatccattctattgggaaagggagacaaaggaaaaatacacatcagagtgaggaaaacctagagtgtggtgccacagttgtatgctaaaacagtttttctgtatcagtttttctgatattgtgtcattgtattggctgttgtcatcttatttttttttattcttttgtctgaaatgtgacattaataagtttatttccttcaaattaatgtctgacggctcggtggttgtataagagcaatcctccgtgttagagacattgggacaatatagtgacattacgaagaaactgactagtaatgcaacagaattacaagagtaacgtatgatgtttgttaattggaatttgactagcaaataattaattgtaaggatatcagcaacccgagcacggcagtcatagatgatgggtgattcaaaaggtgtcactggcactagcctaacggtgttgactaactaggccgcttccctaattagctaacccgggggcgcgccgggacccgaattcattatgtggtc

SEQ ID NO: 4 - DNA, 596 bp, synthetic construct
  source 1..596: mol_type = other DNA
cctgcaggcccgggggcgcgccctaattagctaacggccaggatcgccgcgtgagcctttagcaactagctagattaattaacgcaatctgttattaagttgtctaagcgtcaatttgtttacaccacaatataaaaagtattgtttaattacataagatgtcaagtacactatggcgcctgggttatatttgtgtcaatgtaaatgtttatgagcatttcagtcacacagatcaattattaactagatcttttctacgcactccaactgatgatgtcctcgaacctcaccacagcgtgctacagtaggatagcctcagctcccctatgacagcttcacgaagaatcgttgttagcgtatgccctccacactcaatttccgctacctttcgatttccagcacgagtgaagcatcctcagtcaatgaaagtagaattggcaagcaccagtggtcggttttactggccagcatatggatgtagaattggctacggactacataattgcttgatgccccaccaggcaccagttgggccttttctttatactcccgccatcttcctgaaggatccgaaaggactgctagatgttcatctg

SEQ ID NO: 5 - DNA, 10850 bp, synthetic construct
  source 1..10850: mol_type = other DNA
actggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatata

SEQ ID NO: 6 - DNA, 11792 bp, synthetic construct
  source 1..11792: mol_type = other DNA
caacgcaagctgcataccatcttgtatgatgttctgtagagttgggaaggaaatgatccattctattgggaaagggagacaaaggaaaaatacacatcagagtgaggaaaacctagagtgtggtgccacagttgtatgctaaaacagtttttctgtatcagtttttctgatattgtgtcattgtattggctgttgtcatcttatttttttttattcttttgtctgaaatgtgacattaataagtttatttccttcaaattaatgtctgacggctcggtggttgtataagagcaatcctccgtgttagagacattgggacaatatagtgacattacgaagaaactgactagtaatgcaacagaattacaagagtaacgtatgatgtttgttaattggaatttgactagcaaataattaattgtaaggatatcagcaacccgagcacggcagtcatagatgatgggtgattcaaaaggtgtcactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatataaaaagtattgtttaattacataagatgtcaagtacactatggcgcctgggttatatttgtgtcaatgtaaatgtttatgagcatttcagtcacacagatcaattattaactagatcttttctacgcactccaactgatgatgtcctcgaacctcaccacagcgtgctacagtaggatagcctcagctcccctatgacagcttcacgaagaatcgttgttagcgtatgccctccacactcaatttccgctacctttcgatttccagcacgagtgaagcatcctcagtcaatgaaagtagaattggcaagcaccagtggtcggttttactggccagcatatggatgtagaattggctacggactacataattgcttgatgccccaccaggcaccagttgggccttttctttatactcccgccatcttcctgaaggatccgaaaggactgctagatgttcatctg

SEQ ID NO: 7 - DNA, 10624 bp, synthetic construct
  source 1..10624: mol_type = other DNA
taggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcctaaaccatggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtaggagctcgccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccatactaattagctaacggaccgctaattagctaacgatcgcttaattaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcagggatctccgatcatgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 8 - DNA, 1000 bp, synthetic construct
  source 1..1000: mol_type = other DNA
tttagggcaattggatgttattcccaatggttacaagattatgtacacaaattctaagacacagattttaggcagcgcatgtgatgaggatgtctgacataatgcttccagaggcgggcaggcagcctgcaccacggagagggccactaaaaatcgtcactagaggctgtcccgagacacacaaacaaataaaaatttaaaaagatgggaccaagggaaattgagactctaacttgtgagacataacactcatttacttgccgctccagcaaattaaaaatgaactgtaatcttttagtgacaatgttttggtgccagtcaattaaatatgccgactgctagccaatcttccatgaaagaaatgataattatgaagagtaaatgtgtgcaaaccaaacaagtaatatgccagccatcaatacacaccagctgtactgtgattaaaaaatatgagctccgctgcaactggtaagtataccgcctatatatatagcgttcccacttcacccaaataattcgtcaacgcaagctgcataccatcttgtatgatgttctgtagagttgggaaggaaatgatccattctattgggaaagggagacaaaggaaaaatacacatcagagtgaggaaaacctagagtgtggtgccacagttgtatgctaaaacagtttttctgtatcagtttttctgatattgtgtcattgtattggctgttgtcatcttatttttttttattcttttgtctgaaatgtgacattaataagtttatttccttcaaattaatgtctgacggctcggtggttgtataagagcaatcctccgtgttagagacattgggacaatatagtgacattacgaagaaactgactagtaatgcaacagaattacaagagtaacgtatgatgtttgttaattggaatttgactagcaaataattaattgtaaggatatcagcaacccgagcacggcagtcatagatgatgggtgattcaaaaggtgtc

SEQ ID NO: 9 - DNA, 1000 bp, synthetic construct
  source 1..1000: mol_type = other DNA
aaaagtattgtttaattacataagatgtcaagtacactatggcgcctgggttatatttgtgtcaatgtaaatgtttatgagcatttcagtcacacagatcaattattaactagatcttttctacgcactccaactgatgatgtcctcgaacctcaccacagcgtgctacagtaggatagcctcagctcccctatgacagcttcacgaagaatcgttgttagcgtatgccctccacactcaatttccgctacctttcgatttccagcacgagtgaagcatcctcagtcaatgaaagtagaattggcaagcaccagtggtcggttttactggccagcatatggatgtagaattggctacggactacataattgcttgatgccccaccaggcaccagttgggccttttctttatactcccgccatcttcctgaaggatccgaaaggactgctagatgttcatctgtcaccatgattcttgtggtgggtctgctcaatgactttcgggctccaaaaccatatgttcacaggaccctatttcactactggagcatcaggcttctgtccagcaccatatgttctggttgtcttaaaactggagtaccggatttaaatgtggagttcgtatagaggacattaatactggttggtgtcccgaaccggtataagtactaagtttttagtatcggttgaagacaccaaacagtactaatttaatgtgcacaaatgtcattagtaccggttctagtaacaaattggtacaaaattttaattaattcccagttgaaaataccaactgatactaaagatcttcaagctggcgcaaaaaacaaagatgcttttagtgccggttctagcaaagtaccgatactaaaggctattttccatttctggcagaagttttgggttcctgtccaaattactttagtatcagatcttataaagaaccggtactaaaagtgaagggtttagtacgggttcagtaccagttcttgcctagaacc

SEQ ID NO: 10 - DNA, 88 bp, synthetic construct
  source 1..88: mol_type = other DNA
gttattaagttgtctaagcgtcaatttgtttacaccacaatataaaaagtattgtttaattacataagatgtcaagtacactatggcg

SEQ ID NO: 11 - DNA, 30 bp, synthetic construct
  source 1..30: mol_type = other DNA
gttattaagttgtctaagcgtcaatttgtt

SEQ ID NO: 12 - DNA, 27 bp, synthetic construct
  source 1..27: mol_type = other DNA
cgccatagtgtacttgacatcttatgt

SEQ ID NO: 13 - DNA, 17 bp, synthetic construct
  source 1..17: mol_type = other DNA
caccacaatataaaaag

SEQ ID NO: 14 - DNA, 21 bp, synthetic construct
  source 1..21: mol_type = other DNA
ctccaggctgaagaacagcat

SEQ ID NO: 15 - DNA, 18 bp, synthetic construct
  source 1..18: mol_type = other DNA
gcccaggccgatgttgta

SEQ ID NO: 16 - DNA, 25 bp, synthetic construct
  source 1..25: mol_type = other DNA
actacacgaaccactgcgtgcgctt

SEQ ID NO: 17 - DNA, 25 bp, synthetic construct
  source 1..25: mol_type = other DNA
cagtcatagatgatgggtgattcaa

SEQ ID NO: 18 - DNA, 22 bp, synthetic construct
  source 1..22: mol_type = other DNA
agctaattagggaagcggccta

SEQ ID NO: 19 - DNA, 20 bp, synthetic construct
  source 1..20: mol_type = other DNA
tagcctaacggtgttgacta

SEQ ID NO: 20 - DNA, 25 bp, synthetic construct
  source 1..25: mol_type = other DNA
cgtgagcctttagcaactagctaga

SEQ ID NO: 21 - DNA, 25 bp, synthetic construct
  source 1..25: mol_type = other DNA
gcgccatagtgtacttgacatctta

SEQ ID NO: 22 - DNA, 18 bp, synthetic construct
  source 1..18: mol_type = other DNA
ccgggtgaatcagcgttt

SEQ ID NO: 23 - DNA, 18 bp, synthetic construct
  source 1..18: mol_type = other DNA
gccgtggcctttgacagt

SEQ ID NO: 24 - DNA, 20 bp, synthetic construct
  source 1..20: mol_type = other DNA
tgccgccaacgaatcaccgg

SEQ ID NO: 25 - DNA, 21 bp, synthetic construct
  source 1..21: mol_type = other DNA
gaacgtgtgttgggtttgcat

SEQ ID NO: 26 - DNA, 20 bp, synthetic construct
  source 1..20: mol_type = other DNA
tccagcaatccttgcacctt

SEQ ID NO: 27 - DNA, 24 bp, synthetic construct
  source 1..24: mol_type = other DNA
tgcagcctaaccatgcgcagggta

SEQ ID NO: 28 - DNA, 20 bp, synthetic construct
  source 1..20: mol_type = other DNA
cggctcggtggttgtataag

SEQ ID NO: 29 - DNA, 20 bp, synthetic construct
  source 1..20: mol_type = other DNA
ccgttaggctagtgccagtg

SEQ ID NO: 30 - DNA, 20 bp, synthetic construct
  source 1..20: mol_type = other DNA
acaaggtgtccaacctcgtc

SEQ ID NO: 31 - DNA, 20 bp, synthetic construct
  source 1..20: mol_type = other DNA
ctcagctcgtagcgggtgta

SEQ ID NO: 32 - DNA, 21 bp, synthetic construct
  source 1..21: mol_type = other DNA
cgtgagcctttagcaactagc

SEQ ID NO: 33 - DNA, 20 bp, synthetic construct
  source 1..20: mol_type = other DNA
gtggtgaggttcgaggacat

SEQ ID NO: 34 - DNA, 19 bp, synthetic construct
  source 1..19: mol_type = other DNA
ctttccccaacctcgtgtt

SEQ ID NO: 35 - DNA, 20 bp, synthetic construct
  source 1..20: mol_type = other DNA
aacgctaacagcacggatct

SEQ ID NO: 36 - DNA, 21 bp, synthetic construct
  source 1..21: mol_type = other DNA
tcccttcgttccatgcctaaa

SEQ ID NO: 37 - DNA, 20 bp, synthetic construct
  source 1..20: mol_type = other DNA
actcagtggtattggtcgca

SEQ ID NO: 38 - DNA, 20 bp, synthetic construct
  source 1..20: mol_type = other DNA
tggtgccacagttgtatgct

SEQ ID NO: 39 - DNA, 20 bp, synthetic construct
  source 1..20: mol_type = other DNA
gtcattgagcagacccacca

SEQ ID NO: 40 - DNA, 21 bp, synthetic construct
  source 1..21: mol_type = other DNA
cgactgctagccaatcttcca

SEQ ID NO: 41 - DNA, 20 bp, synthetic construct
  source 1..20: mol_type = other DNA
cgagcacggcagtcatagat

SEQ ID NO: 42 - DNA, 23 bp, synthetic construct
  source 1..23: mol_type = other DNA
ccggtactaatgacatttgtgca

SEQ ID NO: 43 - DNA, 20 bp, synthetic construct
  source 1..20: mol_type = other DNA
ttccagcacgagtgaagcat

SEQ ID NO: 44 - DNA, 20 bp, synthetic construct
  source 1..20: mol_type = other DNA
tcaggaagatggcgggagta

SEQ ID NO: 45 - DNA, 20 bp, synthetic construct
  source 1..20: mol_type = other DNA
atcaggcttctgtccagcac

SEQ ID NO: 46 - DNA, 21 bp, synthetic construct
  source 1..21: mol_type = other DNA
acatcatcagttggagtgcgt

SEQ ID NO: 47 - DNA, 21 bp, synthetic construct
  source 1..21: mol_type = other DNA
cggttgaagacaccaaacagt

SEQ ID NO: 48 - DNA, 22 bp, synthetic construct
  source 1..22: mol_type = other DNA
tgtcccaatgtctctaacacgg

SEQ ID NO: 49 - DNA, 21 bp, synthetic construct
  source 1..21: mol_type = other DNA
tggcagaagttttgggttcct

SEQ ID NO: 50 - DNA, 20 bp, synthetic construct
  source 1..20: mol_type = other DNA
gcagcttgcgttgacgaatt

SEQ ID NO: 51 - DNA, 22 bp, synthetic construct
  source 1..22: mol_type = other DNA
agtctcaatttcccttggtccc

SEQ ID NO: 52 - DNA, 20 bp, synthetic construct
  source 1..20: mol_type = other DNA
tcctcccgtctctcgattcc

SEQ ID NO: 53 - DNA, 20 bp, synthetic construct
  source 1..20: mol_type = other DNA
cgttcaggatctcggactcg

SEQ ID NO: 54 - DNA, 20 bp, synthetic construct
  source 1..20: mol_type = other DNA
tgctgtatgtgccttctgct

SEQ ID NO: 55 - DNA, 20 bp, synthetic construct
  source 1..20: mol_type = other DNA
gtcgtactggctgttgacga

SEQ ID NO: 56 - DNA, 21 bp, synthetic construct
  source 1..21: mol_type = other DNA
ttacgtcgcggagagatggat

SEQ ID NO: 57 - DNA, 20 bp, synthetic construct
  source 1..20: mol_type = other DNA
gtgtccgttagactcgtcgg

SEQ ID NO: 58 - DNA, 28 bp, synthetic construct
  source 1..28: mol_type = other DNA
ttgcaatcccatactaattagctaacgg

SEQ ID NO: 59 - DNA, 20 bp, synthetic construct
  source 1..20: mol_type = other DNA
gcgagaaggcaaaatcgtcc

SEQ ID NO: 60 - DNA, 20 bp, synthetic construct
  source 1..20: mol_type = other DNA
cgccagcctgttgaatatgc

SEQ ID NO: 61 - RNA, 45 nt, synthetic construct
  source 1..45: mol_type = other RNA
taatttctactaagtgtagatacaggctggcattatctactcgaa

SEQ ID NO: 62 - RNA, 45 nt, synthetic construct
  source 1..45: mol_type = other RNA
taatttctactaagtgtagatcataccgtaaagttcagtcaacgc

SEQ ID NO: 63 - DNA, 48 bp, synthetic construct
  source 1..48: mol_type = other DNA
agtgcaaaactatgcctggggcagcaaaactcgaattcactggccgtc

SEQ ID NO: 64 - DNA, 49 bp, synthetic construct
  source 1..49: mol_type = other DNA
cggcgtttaacaggctggcattatctactcatgcaagcttggcgtaatc

SEQ ID NO: 65 - DNA, 23 bp, synthetic construct
  source 1..23: mol_type = other DNA
agcggataacaatttcacacagg

SEQ ID NO: 66 - DNA, 20 bp, synthetic construct
  source 1..20: mol_type = other DNA
gacccgacaaacaagtgcac

SEQ ID NO: 67 - DNA, 20 bp, synthetic construct
  source 1..20: mol_type = other DNA
tctaacggacaccaaccagc

SEQ ID NO: 68 - DNA, 23 bp, synthetic construct
  source 1..23: mol_type = other DNA
cccagtcacgacgttgtaaaacg

SEQ ID NO: 69 - DNA, 1802 bp, synthetic construct
  source 1..1802: mol_type = other DNA
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgctggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcag

SEQ ID NO: 70 - DNA, 1993 bp, synthetic construct
  source 1..1993: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatctggatttttttagccctgccttcatacgctatttatttgcttggtactgtttcttttgtcgttgctcaccctgttgtttggtgttacttctgcag

SEQ ID NO: 71 - DNA, 3510 bp, synthetic construct
  source 1..3510: mol_type = other DNA
atggagatcaacaaccagaaccagtgcgtgccgtacaactgccttaacaaccccgagtccgagatcctgaacgtggccatcttctccagcgagcaggtcgcggagatccacctcaagatcacgcgcctgatcctcgagaacttcctgccgggcggctccttcgctttcggcctgttcgacctcatctggggcatcttcaacgaggaccagtggagcgcgttcctcaggcaggtggaggagctgatcaaccagcgcatcacggagttcgccaggggccaggctatccagcggctggtgggcttcggcaggtcctacgacgagtacatcctggccctcaaggagtgggagaacgaccccgacaacccggccagcaaggagcgcgtgaggacccgcttcaggaccaccgacgacgctctcctgacgggcgtccccctcatggctatcccgggcttcgagctggccaccctctcggtgtacgctcagtcggccaacctgcacctcgccctcctgcgggacgctgtgttcttcggcgagaggtggggcctgacccagacgaacatcaacgacctctactccaggctgaagaacagcatccgcgactacacgaaccactgcgtgcgcttctacaacatcggcctgggcaacctcaacgtcatcaggccggagtactaccgcttccagagggagctgaccatcagcgtgctggacctcgtcgccctgttccccaactacgacatccgcacgtacccgatcccaaccaagtcccagctcacgagggagatctacaccgacccgatcatctcgccgggcgcccaggccggctacaccctgcaggacgtcctgagggagccccacctgatggacttcctgaacaggctcatcatctacaccggcgagtacaggggcatcaggcactgggcgggccacgaggtggagtccagcaggacgggcatgatgaccaacatccgcttcccgctctacggcaccgcggccacggccgagccaacccgcttcatcacgccgtccaccttccccggcctgaacctcttctacaggaccctgtcggctcccatcttccgcgacgagccgggcgcgaacatcatcatccgctacaggacctccctcgtggagggcgtcggcttcatccagccgaacaacggcgagcagctgtaccgcgtgaggggcacgctggacagcctggaccagctcccactggagggcgagtccagcctcaccgagtactcgcacaggctgtgccacgtcaggttcgcccagagcctcaggaacgcggagcccctggactacgccagggtgcccatgttcagctggacccacaggtcggctacccccaccaacaccatcgacccagacgtgatcacgcagatcccgctcgtcaaggcccacaccctgcagtcgggcaccaccgtggtcaagggccccggcttcacgggcggcgacatcctgaggaggacctccggcggcccattcgccttcagcaacgtcaacctcgactggaacctgtcccagcgctacagggcgcgcatcaggtacgccagcaccacgaacctgcgcatgtatgtgaccatcgcgggcgagaggatcttcgccggccagttcaacaagacgatgaacaccggcgacccgctcaccttccagtccttcagctacgcgacgatcgacaccgccttcacgttccccacgaaggcctccagcctgaccgtgggcgccgacaccttctccagcggcaacgaggtctacgtggaccgcttcgagctgatcccggtgacggcgaccttcgaggccgagtacgacctggagaaggcccagaaggcggtcaacgccctcttcacctccagcaaccagatcggcctgaagacggacgtgaccgactaccacatcgacaaggtgtccaacctcgtcgagtgcctgagcgacgagttctgcctcgacgagaagagggagctgtccgagaaggtcaagcacgccaagcgcctctgcgacgagaggaacctcctgcaggacccgaacttcaggggaatcaaccgccagccggacaggggctggaggggcagcaccgacatcaccatccagggcggcgacgacgtgttcaaggagaactacgtcacgctcccgggcaccttcgacgagtgctaccccacgtacctgtaccagaagatcgacgagtccaagctcaaggcctacacccgctacgagctgaggggatacatcgaggacagccaggacctcgagatctacctgatccgctacaacgcgaagcacgagacggtgaacgtccccggcacgggctccctgtggcccctctcggctcagtcgccgatcggcaagtgcggcgagcccaacaggtgcgccacccacctcgagtggaacccggacctggactgctcctgccgggacggcgagaagtgcgctcaccactcccaccacttcagcctggacatcgacgtgggctgcacggacctcaacgaggacctgggcgtgtgggtcatcttcaaaatcaagacgcaggacggccacgctaggctgggcaacctcgagttcctggaggagaagccgctggtgggcgaggctctggccagggtcaagagggcggagaagaagtggcgcgacaagagggagaagctggagctggagacgaacatcgtctacaaggaggccaagaagtccgtggacgcgctcttcgtcaacagccagtacgacaggctgcaggcggacaccaacatcgccatcatccacgccgcggacaagcgcgtgcactccatcagggaggcctacctccccgagctgagcgtgatcccgggcgtcaacgctgccatcttcgaggagctggagggccgcatcttcaccgcctactccctgtacgacgcgaggaacgtcatcaagaacggcgacttcaacaacggcctcagctgctggaacgtgaagggccacgtggacgtcgaggagcagaacaaccaccgctcggtgctggtggtccccgagtgggaggctgaggtcagccaggaggtgcgcgtctgcccgggcaggggatacatcctccgcgtgaccgcgtacaaggagggctacggcgagggctgcgtcacgatccacgagatcgaggacaacaccgacgagctgaagttctccaactgcgtggaggaggagatctacccgaacaacacggtcacctgcaacgactacacggccacccaggaggagtacgagggcacgtacacgtcgaggaacaggggctacgacggcgcttacgagtccaacagctcggtgccggccgactacgctagcgcgtacgaggagaaggcctacacggacggccgcagggacaacacctgcgagtcgaacaggggctacggcgactacacgccgctcccggccggctacgtgaccaaggagctggagtacttcccggagacggacaaggtctggatcgagatcggcgagacggagggcaccttcatcgtggactcagtcgagctgctgctcatggaggagtag

SEQ ID NO: 72 - DNA, 1176 bp, synthetic construct
  source 1..1176: mol_type = other DNA
atgcaaaaactcattaactcagtgcaaaactatgcctggggcagcaaaacggcgttgactgaactttacggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggacgattttgccttctcgctgcacgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaa

SEQ ID NO: 73 - DNA, 1001 bp, synthetic construct
  source 1..1001: mol_type = other DNA
gccaacagtcgttgaagctgctgctgtatctgggttatctagtgtctctgcctttgcccaaggatagtgctgtctttcaaagtatttgtatcgtttgtgtcgtgagtcgtgactgagctggtttcaaggaccagttgtgttctcgttacccaaaactatcgtgcgaccgcatatggcttaatcatgaataaatgttgtttgaatttaaactattcgctgaatattgttgttttttgtcatgtcagttaatgttactaaattggttgccttctaatttttgtttactggtgtttgtcgcaccttatctttttactgtatgtttacttcaggttctggcagtctcattttttgtgactagttaaaacttacagctaaaaaaatgcagtttttaattttaatttgaagtttgattagagctattgatacccggaccatcaggttaggttagttgtgcatagaatcataaatattaatcatgttttctatgaattaagtcaaacttgaaagtctggctgaatatagtttctatgaatcatattgatatacatgtttgattatttgttttgctattagctatttactttggtgaatctatataggcttatgcagaacctttttttttgttctatatatccatatcctagtactcagtagctctatgttttctggagactagtggcttgctttttcgtatgtctaattttttgcttgaccattgcaaaacaaaaattacctagtgtaatctctttttataataatcttgtaatgcgtctacctataggtcaaagtaggttttgtttggaacccttagagctaactgttagctagttgataaattattagctgagttaagctagctaatgaactagttttgatattagctgaggatgtttgaaacctaataattattttttattagctaactatactaaattttagtagagagattccaaacaggagttaacatgggatcagattggctatgcgtttgcaatcccata

SEQ ID NO: 74 - DNA, 1035 bp, synthetic construct
  source 1..1035: mol_type = other DNA
gtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatag

SEQ ID NO: 75 - protein, 1169 aa, synthetic construct
MEINNQNQCVPYNCLNNPESEILNVAIFSSEQVAEIHLKITRLILENFLPGGSFAFGLFDLIWGIFNEDQWSAFLRQVEELINQRITEFARGQAIQRLVGFGRSYDEYILALKEWENDPDNPASKERVRTRFRTTDDALLTGVPLMAIPGFELATLSVYAQSANLHLALLRDAVFFGERWGLTQTNINDLYSRLKNSIRDYTNHCVRFYNIGLGNLNVIRPEYYRFQRELTISVLDLVALFPNYDIRTYPIPTKSQLTREIYTDPIISPGAQAGYTLQDVLREPHLMDFLNRLIIYTGEYRGIRHWAGHEVESSRTGMMTNIRFPLYGTAATAEPTRFITPSTFPGLNLFYRTLSAPIFRDEPGANIIIRYRTSLVEGVGFIQPNNGEQLYRVRGTLDSLDQLPLEGESSLTEYSHRLCHVRFAQSLRNAEPLDYARVPMFSWTHRSATPTNTIDPDVITQIPLVKAHTLQSGTTVVKGPGFTGGDILRRTSGGPFAFSNVNLDWNLSQRYRARIRYASTTNLRMYVTIAGERIFAGQFNKTMNTGDPLTFQSFSYATIDTAFTFPTKASSLTVGADTFSSGNEVYVDRFELIPVTATFEAEYDLEKAQKAVNALFTSSNQIGLKTDVTDYHIDKVSNLVECLSDEFCLDEKRELSEKVKHAKRLCDERNLLQDPNFRGINRQPDRGWRGSTDITIQGGDDVFKENYVTLPGTFDECYPTYLYQKIDESKLKAYTRYELRGYIEDSQDLEIYLIRYNAKHETVNVPGTGSLWPLSAQSPIGKCGEPNRCATHLEWNPDLDCSCRDGEKCAHHSHHFSLDIDVGCTDLNEDLGVWVIFKIKTQDGHARLGNLEFLEEKPLVGEALARVKRAEKKWRDKREKLELETNIVYKEAKKSVDALFVNSQYDRLQADTNIAIIHAADKRVHSIREAYLPELSVIPGVNAAIFEELEGRIFTAYSLYDARNVIKNGDFNNGLSCWNVKGHVDVEEQNNHRSVLVVPEWEAEVSQEVRVCPGRGYILRVTAYKEGYGEGCVTIHEIEDNTDELKFSNCVEEEIYPNNTVTCNDYTATQEEYEGTYTSRNRGYDGAYESNSSVPADYASAYEEKAYTDGRRDNTCESNRGYGDYTPLPAGYVTKELEYFPETDKVWIEIGETEGTFIVDSVELLLMEE

SEQ ID NO: 76 - protein, 391 aa, synthetic construct
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 77 - DNA, 2225 bp, synthetic construct
  source 1..2225: mol_type = other DNA
aaaattacgaatcctcccgtctctcgattccttcatctcgggaacatggccttttagggcaattggatgttattcccaatggttacaagattatgtacacaaattctaagacacagattttaggcagcgcatgtgatgaggatgtctgacataatgcttccagaggcgggcaggcagcctgcaccacggagagggccactaaaaatcgtcactagaggctgtcccgagacacacaaacaaataaaaatttaaaaagatgggaccaagggaaattgagactctaacttgtgagacataacactcatttacttgccgctccagcaaattaaaaatgaactgtaatcttttagtgacaatgttttggtgccagtcaattaaatatgccgactgctagccaatcttccatgaaagaaatgataattatgaagagtaaatgtgtgcaaaccaaacaagtaatatgccagccatcaatacacaccagctgtactgtgattaaaaaatatgagctccgctgcaactggtaagtataccgcctatatatatagcgttcccacttcacccaaataattcgtcaacgcaagctgcataccatcttgtatgatgttctgtagagttgggaaggaaatgatccattctattgggaaagggagacaaaggaaaaatacacatcagagtgaggaaaacctagagtgtggtgccacagttgtatgctaaaacagtttttctgtatcagtttttctgatattgtgtcattgtattggctgttgtcatcttatttttttttattcttttgtctgaaatgtgacattaataagtttatttccttcaaattaatgtctgacggctcggtggttgtataagagcaatcctccgtgttagagacattgggacaatatagtgacattacgaagaaactgactagtaatgcaacagaattacaagagtaacgtatgatgtttgttaattggaatttgactagcaaataattaattgtaaggatatcagcaacccgagcacggcagtcatagatgatgggtgattcaaaaggtgtcaaacgtggatcatgctacgttaattatataaaaagtattgtttaattacataagatgtcaagtacactatggcgcctgggttatatttgtgtcaatgtaaatgtttatgagcatttcagtcacacagatcaattattaactagatcttttctacgcactccaactgatgatgtcctcgaacctcaccacagcgtgctacagtaggatagcctcagctcccctatgacagcttcacgaagaatcgttgttagcgtatgccctccacactcaatttccgctacctttcgatttccagcacgagtgaagcatcctcagtcaatgaaagtagaattggcaagcaccagtggtcggttttactggccagcatatggatgtagaattggctacggactacataattgcttgatgccccaccaggcaccagttgggccttttctttatactcccgccatcttcctgaaggatccgaaaggactgctagatgttcatctgtcaccatgattcttgtggtgggtctgctcaatgactttcgggctccaaaaccatatgttcacaggaccctatttcactactggagcatcaggcttctgtccagcaccatatgttctggttgtcttaaaactggagtaccggatttaaatgtggagttcgtatagaggacattaatactggttggtgtcccgaaccggtataagtactaagtttttagtatcggttgaagacaccaaacagtactaatttaatgtgcacaaatgtcattagtaccggttctagtaacaaattggtacaaaattttaattaattcccagttgaaaataccaactgatactaaagatcttcaagctggcgcaaaaaacaaagatgcttttagtgccggttctagcaaagtaccgatactaaaggctattttccatttctggcagaagttttgggttcctgtccaaattactttagtatcagatcttataaagaaccggtactaaaagtgaagggtttagtacgggttcagtaccagttcttgcctagaaccggtactacatatattttgattcccgtccagaactttcactttcccactcaaatgtctttagtatatgttcttaattagaacctatactaaagtttgcccctataaatatgaccttctccaagtcgttctcaattgtgaccaat

SEQ ID NO: 78 - DNA, 208 bp, synthetic construct
  source 1..208: mol_type = other DNA
ctcggtggttgtataagagcaatcctccgtgttagagacattgggacaatatagtgacattacgaagaaactgactagtaatgcaacagaattacaagagtaacgtatgatgtttgttaattggaatttgactagcaaataattaattgtaaggatatcagcaacccgagcacggcagtcatagatgatgggtgattcaaaaggtgtc

SEQ ID NO: 79 - DNA, 217 bp, synthetic construct
  source 1..217: mol_type = other DNA
aaaagtattgtttaattacataagatgtcaagtacactatggcgcctgggttatatttgtgtcaatgtaaatgtttatgagcatttcagtcacacagatcaattattaactagatcttttctacgcactccaactgatgatgtcctcgaacctcaccacagcgtgctacagtaggatagcctcagctcccctatgacagcttcacgaagaatcgttg